(19) United States
(12) Patent Application Publication
(10) Pub. No.: US 2024/0294523 A1
(43) Pub. Date: Sep. 5, 2024

(54) HETEROCYCLIC LACTAM COMPOUND, AND PREPARATION METHOD THEREFOR AND PHARMACEUTICAL USE THEREOF

(71) Applicants: Genfleet Therapeutics (Shanghai) Inc., Pudong New Area, Shanghai (CN); ZHEJIANG GENFLEET THERAPEUTICS CO., LTD., Shaoxing Binhai New City (CN)

(72) Inventors: Fusheng Zhou, Shanghai (CN); Yingtao LIU, Shanghai (CN); Xiaoming XU, Shanghai (CN); Zhubo LIU, Shanghai (CN); Jiong LAN, Shanghai (CN)

(73) Assignees: Genfleet Therapeutics (Shanghai) Inc., Pudong New Area, Shanghai (CN); ZHEJIANG GENFLEET THERAPEUTICS CO., LTD., Shaoxing Binhai New City (CN)

(21) Appl. No.: 18/568,791

(22) PCT Filed: Jun. 10, 2022
(86) PCT No.: PCT/CN2022/098177

§ 371 Date: Dec. 8, 2023

(30) Foreign Application Priority Data
Jun. 11, 2021 (CN) 202110656890.1

(51) Int. Cl. C07D 471/04; A61K 31/4162; A61K 31/4188; A61K 31/437; A61K 31/4985; A61K 31/55; C07D 487/04

(52) U.S. Cl. CPC C07D 471/04; A61K 31/4162; A61K 31/4188; A61K 31/437; A61K 31/4985; A61K 31/55; C07D 487/04

(57) ABSTRACT

Disclosed is a heterocyclic lactam compound or a pharmaceutically acceptable salt, stereoisomer, solvate or prodrug thereof, as represented in formula (I), wherein the compounds have an inhibitory effect on RIPK1 and the definition of each group in the formula is detailed in the description. In addition, further disclosed are a pharmaceutical composition containing the compound, and the use thereof in the preparation of a drug for treating RIPK1-related diseases or conditions.

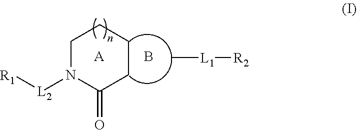

### TECHNICAL FIELD

The present invention relates to the field of medical technology, and in particular to a heterocyclic lactam compound, its application as a RIPK1 inhibitor, and pharmaceutical compositions prepared therefrom.

### BACKGROUND ART

Receptor-interacting protein 1 (RIP1) kinase is a TKL family serine/threonine protein kinase involved in innate immune signaling. RIP1 kinase contains a RHIM domain-containing protein with an N-terminal kinase domain and a C-terminal death domain. The RIP1 death domain mediates interactions with other death domain-containing proteins, which including Fas and TNFR-1, TRAIL-R1 and TRAIL-R2, and TRADD, whereas the RHIM domain are critical for binding other RHIM domain-containing proteins (such as TRIF, DAI and RIP3) and achieve many effects thereof through these interactions.

The role of RIP1 in cell signaling has been evaluated under different conditions (including TLR3, TLR4, TRAIL and FAS), but is best understood in mediating signaling downstream of the death receptor TNFR1.TNFR engagement via TNF results in oligomerization that recruits multiple proteins, including linear K63-linked polyubiquitinated RIP1, TRAF2/5, TRADD, and cIAPs, to the cytoplasmic tail of the receptor. This RIP1-dependent complex serves as a scaffolding protein (i.e., kinase-independent), termed complex I, which provides a platform for pro-survival signaling through activation of the FNκB and MAP kinase pathways. Additionally, under conditions that promote RIP1 deubiquitination, binding of TNF to its receptor (by e.g. inhibition by A20 and CYLD proteins or cIAP) will lead to receptor internalization and formation of complex II or DISC (death-inducing signaling complex) .The formation of DISC (including RIP1, TRADD, FADD and caspase 8) leads to the activation of caspase 8 and also initiates programmed apoptotic cell death in a RIP1 kinase-independent manner ((2012) FEBS J 278,877-887).Apoptosis is a largely quiescent form of cell death that is involved in routine processes such as development and cellular homeostasis. It has been demonstrated by using RIP3 knockout mice (wherein RIP1-mediated necrosis is completely blocked) and necrostatin-1 (a tool inhibitor of RIP1 kinase activity with poor oral bioavailability), that dysregulation of RIP1 kinase-mediated programmed cell death is associated with various inflammations.

RIP3 knockout mice have been shown to be resistant to inflammatory bowel disease (including ulcerative colitis and Crohn's disease), psoriasis, retinal detachment-induced photoreceptor necrosis, retinitis pigmentosa, and bombesin-induced acute pancreatitis and sepsis/systemic inflammatory response syndrome. Necrostatin-1 has been shown to be effective in attenuating ischemic brain injury, retinal ischemia/reperfusion injury, Huntington's disease, renal ischemia-reperfusion injury, cisplatin-induced renal injury, and traumatic brain injury. Other diseases or conditions mediated at least in part by RIP1-dependent apoptosis, necrosis or cytokine production include hematological and solid organ malignancies, bacterial and viral infections (including but not limited to tuberculosis and influenza), and lysosomal storage disease (especially Gaucher disease).

A potent, selective, small molecule inhibitor of RIP1 kinase activity that blocks RIP1-dependent necrosis, thereby providing therapeutic benefits for diseases or events related to DAMPs, cell death and/or inflammation.

### SUMMARY OF THE INVENTION

The present invention provides a heterocyclic lactam compound, which, as a RIPK1 inhibitor, has the advantages of high activity and low toxic and side effects.

In the first aspect, the present invention provides a heterocyclic lactam compound or a pharmaceutically acceptable salt, stereoisomer, solvate or prodrug thereof, the structure of the compound is as shown in formula (I):

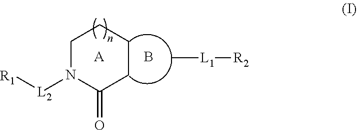

wherein,
R₁ is hydrogen, deuterium, halogen, amino, hydroxyl, cyano, nitro, C₁₋₆ alkyl, C₂₋₆ alkenyl, C₂₋₆ alkynyl, C₁₋₆ alkoxy, C₃₋₁₂ cycloalkyl, 3- to 14-membered heterocycloalkyl, C₆₋₁₄ aryl or 5- to 14-membered heteroaryl; the amino, C₁₋₆ alkyl, C₂₋₆ alkenyl, C₂₋₆ alkynyl, C₁₋₆ alkoxy, C₃₋₁₂ cycloalkyl, 3- to 14-membered heterocycloalkyl, C₆₋₁₄ aryl, 5- to 14-membered heteroaryl group can be optionally substituted by 1, 2, 3 or 4 substituent(s) independently selected from the group S of substituents;
R₂ is C₆₋₁₄ aryl, 5- to 14-membered heteroaryl, C₃₋₁₂ cycloalkyl or 3- to 14-membered heterocycloalkyl; wherein, the C₆₋₁₄ aryl, 5- to 14-membered heteroaryl, C₃₋₁₂ cycloalkyl or 3- to 14-membered heterocycloalkyl can be optionally substituted by 1, 2, 3 or 4 group(s) independently selected from the group S of substituents;
L₁ is one bond, O, —S—, —S(O)—, —SO₂—, —NR₃—, —(C(R₄R₅))ₘ₁—, —C(R₄R₅)—O—, —C(R₄R₅)—S—, —C(R₄R₅)—NH—; wherein m1 is 1 or 2; R₃ is H or C₁₋₆ alkyl; each R₄ and R₅ are the same or different, and are each independently H, hydroxyl, halogen, C₁₋₆ alkyl or halogenated C₁₋₆ alkyl; or R₄ and R₅ on one of the carbon atoms and the carbon atom connected to R₄, R₅ together form a cyclopropyl, cyclobutyl or cyclopentyl group;
L₂ is one bond, —(C(R₆R₇))ₘ₂—, —C(R₆R₇)—C(═O)— or —(C(R₆R₇))ₘ₂—O—; wherein m2 is 1 or 2; each R₆ and R₇ are the same or different, and are each independently H, hydroxyl, halogen, C₁₋₆ alkyl, halogenated C₁₋₆ alkyl or C₃₋₁₂ cycloalkyl; or R₆, R₇ on one of the carbon atoms and the carbon atoms connected to R₆, R₇ together form cyclopropyl, cyclobutyl or cyclopentyl;
the condition is that when R₁ is a C₆₋₁₄ aryl group or a 5- to 14-membered heteroaryl group, L₂ is not one bond;
ring A and ring B are fused to form a bicyclic ring system; n is 0, 1 or 2; ring A is a 5- to 7-membered nitrogen-containing heterocyclic ring; ring B is a 5- to 6-membered heteroaromatic ring; wherein, ring A is optionally substituted by 1, 2, 3 or 4 group(s) independently selected from the group S of substituents; ring B can be optionally substituted by 1, 2, 3 or 4 group(s) independently selected from the group S of substituents;
the group S of substituents include: hydrogen, deuterium, halogen, nitro, cyano, oxo, hydroxyl, carboxyl, C₁₋₆ alkyl, C₂₋₆ alkenyl, C₂₋₆ alkynyl, C₁₋₆ alkoxy, C₁₋₆ alkylthio, C₃₋₁₂ cycloalkyl, C₃₋₁₀ cycloalkenyl, C₆₋₁₄ aryl, 5- to 14-membered heteroaryl, 3- to 14-membered heterocycloalkyl, C₆₋₁₄ aryl-O—, C₆₋₁₄ aryl-C₁₋₄ alkyl-O—, 5- to 14-membered heteroaryl-O—, 3- to 14-membered heterocycloalkyl-O—, C₁₋₆ alkyl-C(═O)O—, C₆₋₁₄ aryl-C(═O)O—, C₁₋₆ alkoxy-C(═O)O—, 5- to 14-membered heteroaryl-C(═O)O—, 3- to 14-membered heterocycloalkyl-C(═O)O—, N(R₈R₉)—C(═O)O—, N(R₈R₉)—C(═O)O—, C₁₋₆ alkyl-sulfonyloxy, C₆₋₁₄ aryl-sulfonyloxy, formyl, C₁₋₆ alkyl-C(═O)—, C₆₋₁₄ aryl-C(═O)—, 5- to 14-membered heteroaryl-C(═O)—, 3- to 14-membered heterocycloalkyl-C(═O)—, C₁₋₆ alkoxy-C(═O)—, C₆₋₁₄ aryl-OC(═O)—, C₆₋₁₄ aryl-C₁₋₄ alkyl-O—C(═O)—, N(R₈R₉)—C(═O)—, N(R₈R₉)-C(═S)—, C₁₋₆ alkylsulfonyl, C₆₋₁₄ arylsulfonyl, 5- to 14-membered heteroarylsulfinyl, C₁₋₆ alkylsulfinyl, C₆₋₁₄ arylsulfinyl, 5- to 14-membered heteroarylsulfinyl, —N(R₈R₉); wherein, R₈ and R₉ are each independently H, C₁₋₆ alkyl, halogenated C₁₋₆ alkyl, C₆₋₁₄ aryl, 5- to 14-membered heteroaryl, 3- to 14-membered heterocycloalkyl, C₆₋₁₄ aryl-C₁₋₄ alkyl-, formyl, C₁₋₆ alkyl-C(═O)—C₆₋₁₄ aryl-C(═O)—, C₁₋₆ alkoxy-C(═O)—, C₆₋₁₄ aryl-C₁₋₄ alkyl-O—C(═O)—, C₁₋₆ alkylsulfonyl, C₆₋₁₄ arylsulfonyl; the C₁₋₆ alkyl, C₂₋₆ alkenyl, C₂₋₆ alkynyl, C₁₋₆ alkoxy, C₁₋₆ alkylthio, C₃₋₁₂ cycloalkyl, C₃₋₁₀ cycloalkenyl, C₆₋₁₄ aryl, 5- to 14-membered heteroaryl, 3- to 14-membered heterocycloalkyl can be optionally substituted by one or more groups selected from the following group: halogen, hydroxyl, cyano, C₁₋₆ alkyl, halogenated C₁₋₆ alkyl.

In one example, the structure of the compound is shown in formula (I-1):

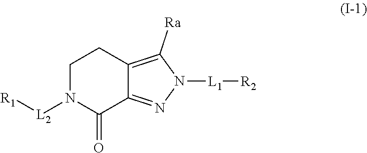

wherein, R₁, L₁, R₂, and L₂ are as defined in formula (I); Ra is selected from the group in the group S of substituents; the group S of substituents are defined as in formula (I).

In one example, Ra is selected from: hydrogen, deuterium, halogen, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl and 3- to 14-membered heterocycloalkyl. Preferably, Ra is selected from: hydrogen, halogen, C₁₋₆ alkyl, C₁₋₆ haloalkyl and C₃₋₆ cycloalkyl. Preferably, Ra is selected from: hydrogen, fluorine, chlorine, bromine, methyl, trifluoromethyl and cyclopropyl.

In one example, the structure of the compound is shown in formula (I-2):

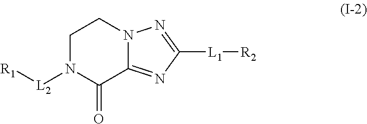

wherein, R₁, L₁, R₂, and L₂ are as defined in formula (I).

In one example, the structure of the compound is shown in formula (I-3):

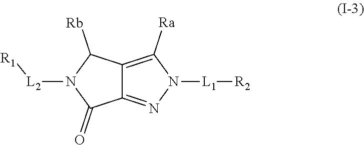

wherein, R₁, L₁, R₂, and L₂ are as defined in formula (I); Ra and Rb are selected from the group in the group S of substituents; the group S of substituents are defined as in formula (I).

In one example, Ra and Rb are each independently selected from: hydrogen, deuterium, halogen, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl and 3- to 14-membered heterocycloalkyl. Preferably, Ra and Rb are each independently selected from: hydrogen, halogen, C₁₋₆ alkyl, C₁₋₆ haloalkyl and C₃₋₆ cycloalkyl. Preferably, Ra and Rb are each independently selected from: hydrogen, fluorine, chlorine, bromine, methyl, trifluoromethyl and cyclopropyl.

In one example, the structure of the compound is shown in formula (I-4):

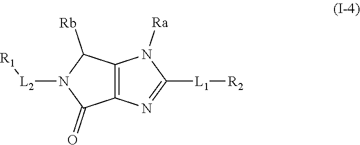

wherein, R₁, L₁, R₂, and L₂ are as defined in formula (I); Ra and Rb are selected from the group in the group S of substituents; the group S of substituents are defined as in formula (I).

In one example, Ra and Rb are each independently selected from: hydrogen, deuterium, halogen, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl and 3- to 14-membered heterocycloalkyl. Preferably, Ra and Rb are each independently selected from: hydrogen, halogen, C₁₋₆ alkyl, C₁₋₆ haloalkyl and C₃₋₆ cycloalkyl. Preferably, Ra and Rb are each independently selected from: hydrogen, fluorine, chlorine, bromine, methyl, trifluoromethyl and cyclopropyl.

In one example, the structure of the compound is shown in formula (I-5):

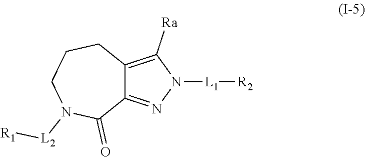

wherein, R₁, L₁, R₂, and L₂ are as defined in formula (I); Ra is selected from the group in the group S of substituents; the group S of substituents are defined as in formula (I).

In one example, Ra is selected from: hydrogen, deuterium, halogen, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl and 3- to 14-membered heterocycloalkyl. Preferably, Ra is selected from: hydrogen, halogen, C₁₋₆ alkyl, C₁₋₆ haloalkyl and C₃₋₆ cycloalkyl. Preferably, Ra is selected from: hydrogen, fluorine, chlorine, bromine, methyl, trifluoromethyl and cyclopropyl.

In one example, when L₂ is one bond, R₁ is a C₃₋₁₂ cycloalkyl group or a 3- to 14-membered heterocycloalkyl group; the C₃₋₁₂ cycloalkyl or 3- to 14-membered heterocycloalkyl can be optionally substituted by 1, 2, 3 or 4 substituent(s) independently selected from the group S of substituents; the group S of substituents are defined as in formula (I).

In one example, when L₂ is one bond, R₁ is piperidine or tetrahydropyran; the piperidine or tetrahydropyran can be optionally substituted by 1 or 2 substituent(s) independently selected from the group S of substituents; the group S of substituents include: hydrogen, deuterium, halogen, nitro, cyano, oxo, hydroxyl, carboxyl, C₁₋₆ alkyl, C₂₋₆ alkenyl, C₂₋₆ alkynyl, C₁₋₆ alkoxy, C₁₋₆ alkylthio, phenyl.

In one example, when L₂ is —(C(R₆R₇))ₘ₂—, —C(R₆R₇)—C(═O)— or (C(R₆R₇))ₘ₂—O—, R₁ is hydrogen, deuterium, halogen, amino, hydroxyl, cyano, nitro, C₁₋₆ alkyl, C₂₋₆ alkenyl, C₂₋₆ alkynyl, C₁₋₆ alkoxy, C₃₋₁₂ cycloalkyl, 3- to 14-membered heterocycloalkyl, C₆₋₁₄ aryl or 5- to 14-membered heteroaryl; the amino, C₁₋₆ alkyl, C₂₋₆ alkenyl, C₂₋₆ alkynyl, C₁₋₆ alkoxy, C₃₋₁₂ cycloalkyl, 3- to 14-membered heterocycloalkyl, C₆₋₁₄ aryl, 5- to 14-membered heteroaryl can be optionally substituted by 1, 2, 3 or 4 substituent(s) independently selected from the group S of substituents; R₆, R₇, m2, and group S of substituents are each defined as in formula (I).

In one example, when L₂ is —(C(R₆R₇))ₘ₂—, —C(R₆R₇)—C(═O)— or (C(R₆R₇))ₘ₂—O—, R₁ is hydrogen, deuterium, halogen, amino, hydroxyl, cyano, C₁₋₆ alkyl, C₂₋₆ alkenyl, C₂₋₆ alkynyl, C₁₋₆ alkoxy, phenyl, tetrahydropyranyl, azetidinyl, piperidyl or pyridyl; the amino, C₁₋₆ alkyl, C₂₋₆ alkenyl, C₂₋₆ alkynyl, C₁₋₆ alkoxy, phenyl, tetrahydropyranyl, azetidinyl, piperidyl or pyridyl can be optionally substituted by 1, 2, 3 or 4 substituent(s) independently selected from the group S of substituents; R₆, R₇, m2, and group S of substituents are each defined as in formula (I).

In one example, when L₂ is —(C(R₆R₇))ₘ₂—, —C(R₆R₇)—C(═O)— or (C(R₆R₇))ₘ₂—O—, R₁ is hydrogen, deuterium, halogen, amino, hydroxyl, cyano, C₁₋₆ alkyl, C₂₋₆ alkenyl, C₂₋₆ alkynyl, C₁₋₆ alkoxy, phenyl, tetrahydropyranyl, azetidinyl, piperidyl or pyridyl; the amino, C₁₋₆ alkyl, C₂₋₆ alkenyl, C₂₋₆ alkynyl, C₁₋₆ alkoxy, phenyl, tetrahydropyranyl, azetidinyl, piperidyl or pyridyl can be optionally substituted by 1 or 2 substituent(s) independently selected from the group S of substituents; the group S of substituents include: hydrogen, deuterium, halogen, nitro, cyano, oxo, hydroxyl, carboxyl, C₁₋₆ alkyl, C₂₋₆ alkenyl, C₂₋₆ alkynyl, C₁₋₆ alkoxy, C₁₋₆ alkylthio; R₆, R₇ and m2 are each defined as in formula (I).

In one example, when L₂ is —(C(R₆R₇))ₘ₂—, —C(R₆R₇)—C(═O)— or —(C(R₆R₇))ₘ₂—O—, R₁ is hydrogen, cyano, methyl, ethyl, difluoromethyl, trifluoromethyl, methoxy, isopropyl, phenyl, tetrahydropyran, difluoroazetidinyl, piperidyl, pyridyl, 4-cyanopyridyl; R₆, R₇ and m2 are each defined as in formula (I).

In one example, when L₂ is —(C(R₆R₇))ₘ₂—, L₂ is —CH₂—, —CH₂CH₂—, —CH(CH₃)—, —C(CH₃)₂—, —CH(CH₂CH₃)—, —CH₂CH(CH₃)—, —CH(CH₃)CH₂—, —CH₂CH(CH₂CH₃)—, —CH(CH₂CH₃)CH₂—, —CH(cyclopropyl)-, CH₂CH(cyclopropyl)-, —CH(cyclopropyl)CH₂—.

In one example, when L₂ is —C(R₆R₇)—C(═O)—, L₂ is —C(═O)— or —CH₂C(═O)—.

In one example, when L₂ is —(C(R₆R₇))ₘ₂—, L₂ is —CR₆R₇- or —CH₂—CR₆R₇-; wherein R₆, R₇ and the carbon atoms connected to R_{6,} R₇ together form cyclopropyl, cyclobutyl or cyclopentyl.

In one example, when L₂ is —(C(R₆R₇))ₘ₂—O—, L₂ is —CH₂—O—, —CH₂CH₂—O—, —CH(CH₃)—O—, —C(CH₃)₂—O—, —CH(CH₂CH₃)—O—, —CH₂CH(CH₃)—O—, —CH(CH₃)CH₂—O—, —CH₂CH(CH₂CH₃)—O—, —CH(CH₂CH₃)CH₂—O—, —CH(cyclopropyl)-O—, —CH₂CH(cyclopropyl)-O—, —CH(cyclopropyl)CH₂—O—.

In one example, ring A is piperidine, pyrrolidine, pyrroline, piperazine, tetrahydropyridine or azepane; and ring A can be optionally substituted by 1, 2, 3 or 4 substituent(s) independently selected from the group S of substituents; the group S substituent are defined as in formula (I).

In one example, ring B is 5- to 6-membered nitrogen-containing monocyclic heteroaromatic ring; and ring B can be optionally substituted by 1, 2, 3 or 4 substituent(s) independently selected from the group S of substituents; the group S substituent are defined as in formula (I).

In one example, ring B is pyrazole, triazole, imidazole, thiazole, isothiazole, oxazole, isoxazole or pyridine; and ring B can be optionally substituted by 1, 2, 3 or 4 substituent(s) independently selected from the group S of substituents; the group S substituent are defined as in formula (I).

In one example, ring B is pyrazole, triazole or imidazole; and ring B can be optionally substituted by 1 or 2 substituent(s) independently selected from the group S of substituents. The group S of substituents include: deuterium, halogen, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl, 3- to 14-membered heterocycloalkyl; preferably, the group S of substituents include: halogen, C₁₋₆ alkyl, C₁₋₆ haloalkyl, C₃₋₆ cycloalkyl.

In one example, ring A and ring B are fused to form a bicyclic ring system; the bicyclic ring system is selected from the group consisting of:

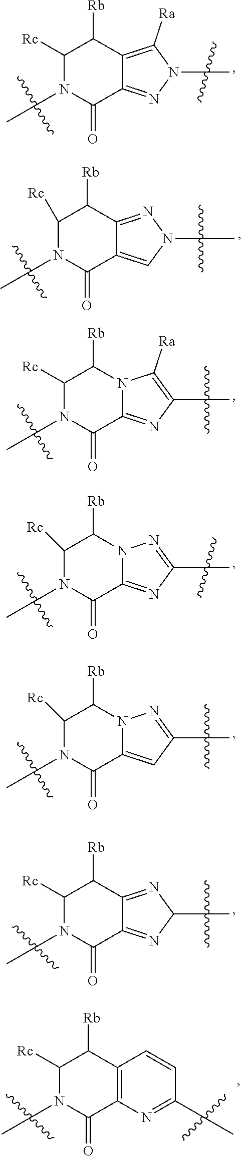

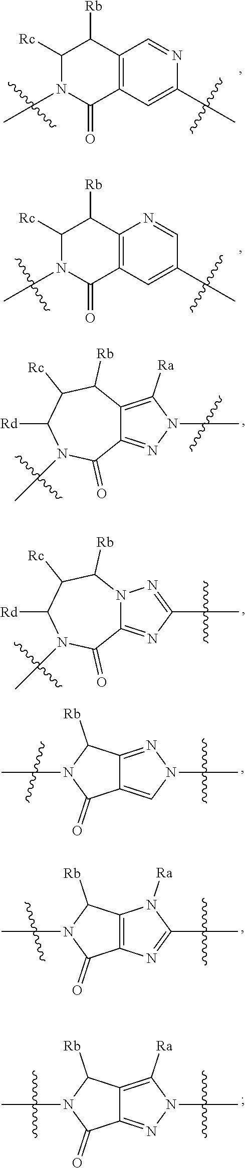

wherein Ra, Rb, Rc, Rd are groups each independently selected from the group S of substituents; the group S of substituents are defined as in formula (I).

In one example, Ra, Rb, Rc, Rd are each independently selected from: hydrogen, deuterium, halogen, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl, 3- to 14-membered heterocycloalkyl. Preferably, Ra, Rb, Rc, Rd are each independently selected from: hydrogen, halogen, C₁₋₆ alkyl, C₁₋₆ haloalkyl and C₃₋₆ cycloalkyl. Preferably, Ra, Rb, Rc, Rd are each independently selected from: hydrogen, fluorine, chlorine, bromine, methyl, trifluoromethyl, and cyclopropyl.

In one example, R₂ is C₆₋₁₄ aryl; the C₆₋₁₄ aryl is phenyl, naphthyl, or 9- or 10-membered aromatic fused bicyclic ring formed by the fusion of phenyl and a non-aromatic ring; the non-aromatic ring is a 3- to 6-membered saturated or partially unsaturated monocyclic heterocycloalkyl group or a 3- to 6-membered saturated or partially unsaturated monocyclic cycloalkyl group; wherein the 3- to 6-membered saturated or partially unsaturated monocyclic heterocycloalkyl is selected from: aziridine, oxirane, azetidine, azetidin-2-one, oxetane, oxetan-2-one, oxazolidine, pyrrolidin-2-one, pyrrolidin-2,5-dione, 1,3-dioxolane, dihydrofuran-2(3H)-one, dihydrofuran-2,5-dione, piperidin-2-one, piperidin-2,6-dione, tetrahyro-2H-pyran-2-one, imidazolidine, tetrahydrofuran, tetrahydrothiophene, tetrahydropyrrole, 1,3-dioxolan-2-one, oxazolidin-2-one, imidazolidine-2-one, piperidine, piperazine, piperazin-2-one, morpholine, morpholin-3-one, morpholin-2-one, thiomorpholin-3-one 1,1-dioxide, thiomorpholine, thiomorpholine-1,1-dioxide, tetrahydropyran, 1,2-dihydroazacyclobutadiene, 1,2-dihydrooxetadiene, 2,5-dihydro-1H-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, 1,3-oxazinane, hexahydropyrimidine, 1,4-dioxane, tetrahydropyrimidin-2(1H)-one, 1,4-dioxan-2-one, 5,6-dihydro-2H-pyran-2-one; the 3- to 6-membered saturated or partially unsaturated monocyclic cycloalkyl is selected from cyclopropyl, cyclobutyl, cyclopentyl, cyclopentenyl, cyclohexyl, cyclohexenyl, cyclohexdienyl, cyclobutanone, cyclobutan-1,2-dione, cyclopentanone, cyclopentan-1,3-dione, cyclohexanone, cyclohexan-1,3-dione; the phenyl, naphthyl or 9- or 10-membered aromatic fused bicyclic ring is unsubstituted or substituted by 1, 2, 3 or 4 substituent(s) each independently selected from the group S; the group S substituents are defined as in formula (I).

In one example, R₂ is phenyl; the phenyl is unsubstituted or substituted by 1, 2, 3 or 4 substituent(s) each independently selected from the group S of substituents; the group S of substituents are defined as in formula (I).

In one example, R₂ is phenyl; the phenyl is unsubstituted or substituted by 1, 2, 3 or 4 substituent(s) each independently selected from the group S of substituents; the group S of substituents include: halogen, cyano, hydroxyl, carboxyl, C₁₋₆ alkyl, C₂₋₆ alkenyl, C₂₋₆ alkynyl, C₁₋₆ alkoxy, C₁₋₆ alkylthio, halogenated C₁₋₆ alkyl, halogenated C₂₋₆ alkenyl, halogenated C₂₋₆ alkynyl, halogenated C₁₋₆ alkoxy and halogenated C₁₋₆ alkylthio.

In one example, R₂ is phenyl; the phenyl is unsubstituted or substituted by 1, 2 or 3 substituent(s) each independently selected from the group S of substituents; the group S of substituents include: halogen, cyano, hydroxyl, carboxyl, methyl, methoxy, trifluoromethyl and trifluoromethoxy.

In one example, R₂ is phenyl, 2-substitutent-phenyl, 3-substitutent-phenyl, 4-substitutent-phenyl, 2,3-disubstitutent-phenyl, 2,4-disubstitutent-phenyl, 2,5-disubstitutent-phenyl, 2,6-disubstitutent-phenyl, 3,4-disubstitutent-phenyl, 3,5-disubstitutent-phenyl, 3,6-disubstitutent-phenyl, 2,3,4-trisubstitutent-phenyl, 2,3,5-trisubstitutent-phenyl, 2,3,6-trisubstitutent-phenyl, 2,4,5-tri substituent-phenyl, 2,4,6-trisubstitutent-phenyl, 2,5,6-trisubstitutent-phenyl; the substituents are each independently selected from the group S of substituents; the group S of substituents include: halogen, cyano, hydroxyl, carboxyl, C₁₋₆ alkyl, C₂₋₆ alkenyl, C₂₋₆ alkynyl, C₁₋₆ alkoxy, C₁₋₆ alkylthio, halogenated C₁₋₆ alkyl, halogenated C₂₋₆ alkenyl, halogenated C₂₋₆ alkynyl, halogenated C₁₋₆ alkoxy, halogenated C₁₋₆ alkylthio. Preferably, the group S of substituents include: halogen, cyano, hydroxyl, carboxyl, methyl, methoxy, trifluoromethyl and trifluoromethoxy.

In one example, R₂ is phenyl, 2-fluorophenyl, 3-fluorophenyl, 4-fluorophenyl, 2,3-difluorophenyl, 2,4-difluorophenyl, 2,6-difluorophenyl, 2,5-difluorophenyl, 3,4-difluorophenyl, 3,5-difluorophenyl, 2,4,6-trifluorophenyl, 2,4,5-trifluorophenyl, 2,5,6-trifluorophenyl, 2-chlorophenyl, 3-chlorophenyl, 4-chlorophenyl, 2,3-dichlorophenyl, 2,4-dichlorophenyl, 2,6-dichlorophenyl, 3,5-dichlorophenyl, 2,5-dichlorophenyl, 2,4,6-trichlorophenyl, 2,4,5-trichlorophenyl, 2,5,6-trichlorophenyl, 2-trifluoromethoxyphenyl, 2-cyanophenyl, 3-cyanophenyl, 4-cyanophenyl, 2-methylphenyl, 3-methylphenyl, 4-methylphenyl, 2-amidophenyl, 3-amidophenyl, 4-amidophenyl, 2-fluoro-3-methyl-phenyl, 2-fluoro-4-methyl-phenyl, 2-fluoro-5-methyl-phenyl, 2-fluoro-6-methyl-phenyl, 2-fluoro-3-cyano-phenyl, 2-fluoro-4-cyano-phenyl, 2-fluoro-5-cyano-phenyl, 2-fluoro-6-cyano-phenyl, 3-fluoro-2-methyl-phenyl, 3-fluoro-4-methyl-phenyl, 3-fluoro-5-methyl-phenyl, 3-fluoro-6-methyl-phenyl, 3-fluoro-2-cyano-phenyl, 3-fluoro-4-cyano-phenyl, 3-fluoro-5-cyano-phenyl, 3-fluoro-6-cyano-phenyl, 2-chloro-3-methyl-phenyl, 2-chloro-4-methyl-phenyl, 2-chloro-5-methyl-phenyl, 2-chloro-6-methyl-phenyl, 2-chloro-3-cyano-phenyl, 2-chloro-4-cyano-phenyl, 2-chloro-5-cyano-phenyl, 2-chloro-6-cyano-phenyl, 3-chloro-2-methyl-phenyl, 3-chloro-4-methyl-phenyl, 3-chloro-5-methyl-phenyl, 3-chloro-6-methyl-phenyl, 3-chloro-2-cyano-phenyl, 3-chloro-4-cyano-phenyl, 3-chloro-5-cyano-phenyl, 3-chloro-6-cyano-phenyl, 2-methylsulfonylphenyl, 3-methylsulfonylphenyl, 4-methylsulfonylphenyl.

In one example, R₂ is 5- to 14-membered heteroaryl; the 5- to 14-membered heteroaryl is a 5- or 6-membered monocyclic heteroaryl; the 5- or 6-membered monocyclic heteroaryl is unsubstituted or substituted by 1, 2, 3 or 4 substituent(s) each independently selected from the group S of substituents; the group S of substituents are defined as in formula (I).

In one example, R₂ is 5- or 6-membered monocyclic heteroaryl; the 5- or 6-membered monocyclic heteroaryl is selected from the group of: thiophene, N-alkylpyrrolidone, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine or pyrazine; the 5- or 6-membered monocyclic heteroaryl is unsubstituted or substituted by 1, 2, 3 or 4 substituent(s) each independently selected from the group S of substituents; the group S of substituents are defined as in formula (I). Preferably, the group S substituents include: hydrogen, deuterium, halogen, cyano, hydroxyl, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl, 3- to 14-membered heterocycloalkyl. Preferably, the group S substituents include: hydrogen, halogen, cyano, hydroxyl, C₁₋₆ alkyl, C₁₋₆ haloalkyl, C₃₋₆ cycloalkyl. Preferably, the group S substituents include: hydrogen, fluorine, chlorine, bromine, cyano, hydroxyl, methyl, trifluoromethyl and cyclopropyl.

In one example, R₂ is 5- or 6-membered monocyclic heteroaryl; the 5- or 6-membered monocyclic heteroaryl is selected from the group of: furan, oxazole, pyridine, pyrimidine or pyrazole; the 5- or 6-membered monocyclic heteroaryl is unsubstituted or substituted by 1, 2, 3 or 4 substituent(s) each independently selected from the group S of substituents; the group S of substituents are defined as in formula (I). Preferably, the group S substituents include: hydrogen, deuterium, halogen, cyano, hydroxyl, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl, 3- to 14-membered heterocycloalkyl. Preferably, the group S substituents include: hydrogen, halogen, cyano, hydroxyl, C₁₋₆ alkyl, C₁₋₆ haloalkyl, C₃₋₆ cycloalkyl. Preferably, the group S substituents include: hydrogen, fluorine, chlorine, bromine, cyano, hydroxyl, methyl, trifluoromethyl and cyclopropyl.

In one example, R₂ is 5- or 6-membered monocyclic heteroaryl; the 5- or 6-membered monocyclic heteroaryl is selected from the group of: pyridin-2-yl-, pyridin-3-yl-, pyridin-4-yl-, pyrimidin-2-yl-, pyrazol-3-yl-, pyrazol-4-yl-, pyrazol-5-yl-; the 5- or 6-membered monocyclic heteroaryl is unsubstituted or substituted by 1 or 2 substituent(s) each independently selected from the group S of substituents; the group S of substituents are defined as in formula (I). Preferably, the group S substituents include: hydrogen, deuterium, halogen, cyano, hydroxyl, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl, 3- to 14-membered heterocycloalkyl. Preferably, the group S substituents include: hydrogen, halogen, cyano, hydroxyl, C₁₋₆ alkyl, C₁₋₆ haloalkyl, C₃₋₆ cycloalkyl. Preferably, the group S substituents include: hydrogen, fluorine, chlorine, bromine, cyano, hydroxyl, methyl, trifluoromethyl and cyclopropyl. In one embodiment, R₂ is selected from pyridin-2-yl-, pyridin-3-yl-, pyridin-4-yl-, pyrimidinyl, pyrazolyl, pyrazol-3-yl-, pyrazol-4-yl-, pyrazol-5-yl-, 1-methyl-pyrazol-4-yl-, 1-methyl-pyrazol-3-yl-, 1-methyl-pyrazol-5-yl-, 3-fluoro-pyridin-2-yl-, 4-fluoro-pyridin-2-yl-, 5-fluoro-pyridin-2-yl-, 6-fluoro-pyridin-2-yl-, 2-fluoro-pyridin-3-yl-, 4-fluoro-pyridin-3-yl-, 5-fluoro-pyridin-3-yl-, 6-fluoro-pyridin-3-yl-, 3-chloro-pyridin-2-yl-, 4-chloro-pyridine-2-yl-, 5-chloro-pyridin-2-yl-, 6-chloro-pyridin-2-yl-, 2-chloro-pyridin-3-yl-, 4-chloro-pyridin-3-yl-, 5-chloro-pyridin-3-yl-, 6-chloro-pyridin-3-yl-, 3-methyl-pyridin-2-yl-, 4-methyl-pyridin-2-yl-, 5-methyl-pyridin-2-yl-, 6-methyl-pyridin-2-yl-, 2-methyl-pyridin-3-yl-, 4-methyl-pyridin-3-yl-, 5-methyl-pyridin-3-yl-, 6-methyl-pyridin-3-yl-, 3-fluoro-pyrimidin-2-yl-, 5-fluoro-pyrimidin-2-yl-, 6-fluoro-pyrimidin-2-yl-, 3-methyl-pyrimidin-2-yl-, 5-methyl-pyrimidin-2-yl-, 6-methyl-pyrimidin-2-yl-, 3-methyl-pyrimidin-2-yl-, 5-methyl-pyrimidin-2-yl-, 6-methyl-pyrimidin-2-yl-, 3-methyl-pyrimidin-2-yl-, 5-methyl-pyrimidin-2-yl-, 6-methyl-pyrimidine-2-yl-.

In one example, R₂ is 5- to 14-membered heteroaryl; the 5- to 14-membered heteroaryl is a 9- or 10-membered bicyclic heteroaryl formed by the fusion of phenyl and a 5- or 6-membered monocyclic heteroaryl group; the 9- or 10-membered bicyclic heteroaryl selected from the group of: benzoxazole, benzisoxazole, benzimidazole, benzothiazole, benzisothiazole, benzotriazole, benzofuran, benzothiophene, indole, indazole, isoindole, quinoline, isoquinoline, quinazoline, quinoxaline, cinnoline; the 9- or 10-membered bicyclic heteroary is unsubstituted or substituted by 1, 2, 3 or 4 substituent(s) each independently selected from the group S of substituents; the group S of substituents are defined as in formula (I).

In one example, R₂ is 5- to 14-membered heteroaryl; the 5- to 14-membered heteroaryl is a 8- to 10-membered bicyclic heteroaryl formed by the fusion of a 5- or 6-membered monocyclic heteroaryl group and a 5- or 6-membered monocyclic heteroaryl group; the 8- to 10-membered bicyclic heteroaryl is unsubstituted or substituted by 1, 2, 3 or 4 substituent(s) each independently selected from the group S of substituents; the group S of substituents are defined as in formula (I).

In one example, the 8- to 10-membered bicyclic heteroaryl formed by the fusion of a 5- or 6-membered monocyclic heteroaryl group and a 5- or 6-membered monocyclic heteroaryl group is selected from: pyrido[3,2-d]pyrimidine, pyrido[2,3-d]pyrimidine, pyrido[3,4-d]pyrimidine, pyrido[4,3-d]pyrimidine, 1,8-naphthyridine , 1,7-naphthyridine, 1,6-naphthyridine, 1,5-naphthyridine; the 8- to 10-membered bicyclic heteroaryl is unsubstituted or substituted by 1, 2, 3 or 4 substituent(s) each independently selected from the group S of substituents; the group S of substituents are defined as in formula (I).

In one example, R₂ is 5- to 14-membered heteroaryl; the 5- to 14-membered heteroaryl is a 8- to 10-membered bicyclic heteroaryl formed by the fusion of a 5- or 6-membered monocyclic heteroaryl group and a non-aromatic ring; the 8- to 10-membered bicyclic heteroaryl is unsubstituted or substituted by 1, 2, 3 or 4 substituent(s) each independently selected from the group S of substituents; the group S of substituents are defined as in formula (I); the non-aromatic ring is a 3- to 6-membered saturated or partially unsaturated monocyclic heterocycloalkyl group or a 3- to 6-membered saturated or partially unsaturated monocyclic cycloalkyl group; wherein the 3- to 6-membered saturated or partially unsaturated monocyclic heterocycloalkyl is selected from aziridine, oxirane, azetidine, azetidin-2-one, oxetane, oxetan-2-one, oxazolidine, pyrrolidin-2-one, pyrrolidin-2,5-dione, 1,3-dioxolane, dihydrofuran-2(3H)-one, dihydrofuran-2,5-dione, piperidin-2-one, piperidin-2,6-dione, tetrahyro-2H-pyran-2-one, imidazolidine, tetrahydrofuran, tetrahydrothiophene, tetrahydropyrrole, 1,3-dioxolan-2-one, oxazolidin-2-one, imidazolidine-2-one, piperidine, piperazine, piperazin-2-one, morpholine, morpholin-3-one, morpholin-2-one, thiomorpholin-3-one 1,1-dioxide, thiomorpholine, thiomorpholine-1,1-dioxide, tetrahydropyran, 1,2-dihydroazacyclobutadiene, 1,2-dihydrooxetadiene, 2,5-dihydro-1H-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, 1,3-oxazinane, hexahydropyrimidine, 1,4-dioxane, tetrahydropyrimidin-2(1H)-one, 1,4-dioxan-2-one, 5,6-dihydro-2H-pyran-2-one; the 3- to 6-membered saturated or partially unsaturated monocyclic cycloalkyl is selected from cyclopropyl, cyclobutyl, cyclopentyl, cyclopentenyl, cyclohexyl, cyclohexenyl, cyclohexdienyl, cyclobutanone, cyclobutan-1,2-dione, cyclopentanone, cyclopentan-1,3-dione, cyclohexanone, cyclohexan-1,3-dione

In one example, the 8- to 10-membered bicyclic heteroaryl is selected from: benzoxazole, benzisoxazole, benzimidazole, benzothiazole, benzisothiazole, benzotriazole, benzofuran, benzothiophene, indole, indazole, isoindole, quinoline, isoquinoline, quinazoline, quinoxaline, cinnoline, pyridopyrimidine and naphthyridine.

In one example, R₂ is a 5- to 6-membered heterocycloalkyl; the 5- to 6-membered heterocycloalkyl is unsubstituted or substituted by 1, 2, 3 or 4 substituent(s) each independently selected from the group S of substituents; the group S of substituents are defined as in formula (I).

In one example, R₂ is a 5- to 6-membered heterocycloalkyl; the 5- to 6-membered heterocycloalkyl is selected from oxazolidine, pyrrolidin-2-one, pyrrolidin-2,5-dione, 1,3-dioxolane, dihydrofuran-2(3H)-one, dihydrofuran-2,5-dione, piperidin-2-one, piperidin-2,6-dione, tetrahyro-2H-pyran-2-one, imidazolidine, tetrahydrofuran, tetrahydrothiophene, tetrahydropyrrole, 1,3-dioxolan-2-one, oxazolidin-2-one, imidazolidine-2-one, piperidine, piperazine, piperazin-2-one, morpholine, morpholin-3-one, morpholin-2-one, thiomorpholin-3-one 1,1-dioxide, thiomorpholine, thiomorpholine-1,1-dioxide, tetrahydropyran, 1,2-dihydroazacyclobutadiene, 1,2-dihydrooxetadiene, 2,5-dihydro-1H-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, 1,3-oxazinane, hexahydropyrimidine, 1,4-dioxane, tetrahydropyrimidin-2(1H)-one, 1,4-dioxan-2-one, 5,6-dihydro-2H-pyran-2-one; the 5- to 6-membered heterocycloalkyl is unsubstituted or substituted by 1, 2, 3 or 4 substituent(s) each independently selected from the group S of substituents; the group S of substituents are defined as in formula (I).

In one example, R₂ is a 5- to 6-membered heterocycloalkyl; the 5- to 6-membered heterocycloalkyl is selected from tetrahydropyran, tetrahydrofuran, tetrahydro-2H-thiopyran 1,1-dioxide, tetrahydrothiophene 1,1-dioxide, oxetane, 1,4-dioxane; the 5- to 6-membered heterocycloalkyl is unsubstituted or substituted by 1, 2, 3 or 4 substituent(s) each independently selected from the group S of substituents; the group S of substituents are defined as in formula (I).

Preferably, the group S substituents include: hydrogen, deuterium, halogen, cyano, hydroxyl, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl, 3- to 14-membered heterocycloalkyl. Preferably, the group S substituents include: hydrogen, halogen, cyano, hydroxyl, C₁₋₆ alkyl, C₁₋₆ haloalkyl, C₃₋₆ cycloalkyl. Preferably, the group S substituents include: hydrogen, fluorine, chlorine, bromine, cyano, hydroxyl, methyl, trifluoromethyl and cyclopropyl.

In one example, R₂ is a C₃₋₁₂ cycloalkyl; the C₃₋₁₂ cycloalkyl is selected from the group consisting of cyclopropyl, cyclobutyl, cyclopentyl, cyclohexyl; the cyclopropyl, cyclobutyl, cyclopentyl, cyclohexyl is unsubstituted or substituted by 1, 2, 3 or 4 substituent(s) each independently selected from the group S of substituents.

In one example, R₂ is a C₃₋₁₂ cycloalkyl; the C₃₋₁₂ cycloalkyl is selected from the group consisting of cyclopropyl, cyclobutyl, cyclopentyl, cyclohexyl; the cyclopropyl, cyclobutyl, cyclopentyl , cyclohexyl is unsubstituted or substituted by 1 or 2 substituent(s) each independently selected from the group S of substituents; the group S substituents include: hydrogen, deuterium, halogen, cyano, hydroxyl, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl, 3- to 14-membered heterocycloalkyl. Preferably, the group S substituents include: hydrogen, halogen, cyano, hydroxyl, C₁₋₆ alkyl, C₁₋₆ haloalkyl, C₃₋₆ cycloalkyl. Preferably, the group S substituents include: hydrogen, fluorine, chlorine, bromine, cyano, hydroxyl, methyl, trifluoromethyl and cyclopropyl.

As used herein, “substituted” represent that the substitution can take place at any position of the group that can be substituted, such as the 1-position, 2-position, 3-position, 4-position, etc.

In one example, L₁ is —CH₂—, —CH₂CH₂—, —CH(CF₃)— or —CH(CH₃)—.

In one example, R₁-L₂ are selected from the group consisting of:

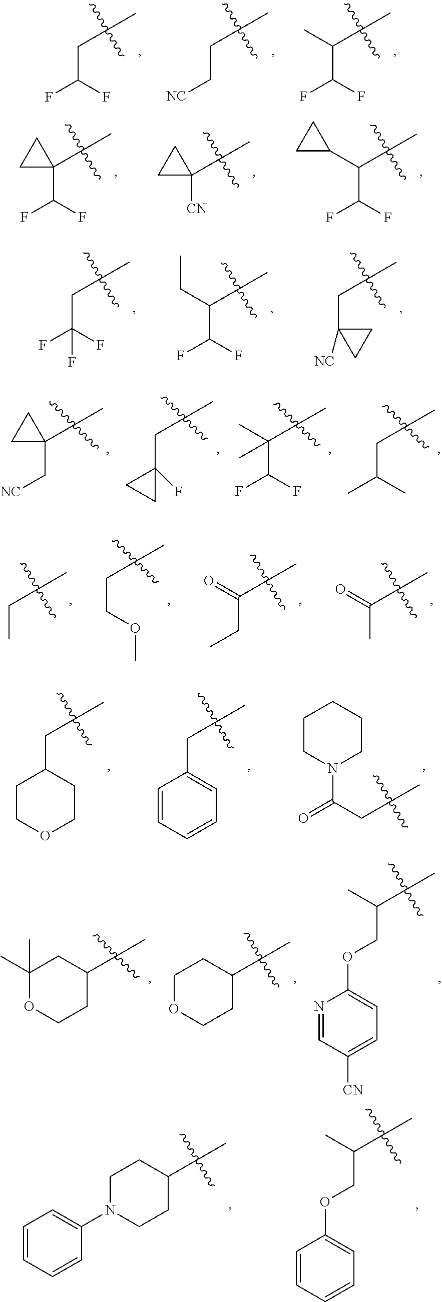

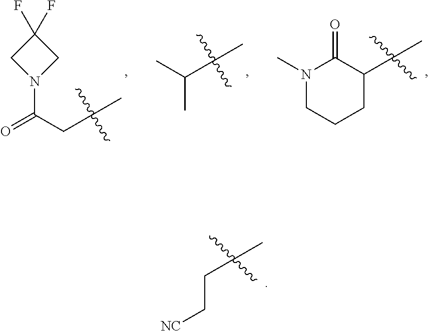

In one example, the group S substituents include: hydrogen, deuterium, halogen, nitro, cyano, oxo, hydroxyl, carboxyl, optionally halogenated C₁₋₆ alkyl, C₂₋₆ alkenyl, C₂₋₆ alkynyl, C₃₋₁₀ cycloalkyl, C₃₋₁₀ cycloalkenyl, C₆₋₁₄ aryl, optionally halogenated C₁₋₆ alkoxy, phenyl-O—, naphthyl-O—, benzyl-O—, pyridyl-O—, morpholinyl-O—, piperidyl-O—, ethyl-C(═O)O—, propyl-C(═O)O—, phenyl-C(═O)O—, 1-naphthyl-C(═O)O—, 2-naphthyl-C(═O)O—, methoxy-C(═O)O—, ethoxy-C(═O)O—, propoxy-C (═O)O—, butoxy-C(═O)O—, methylamino-C(═O)O—, ethylamino-C(═O)O—, dimethylamino-C(═O)O—, diethylamino-C(═O)O—, phenylamino-C(═O)O—, naphthylamino-C(═O)O—, nicotinyloxy, morpholinyl-C(═O)O—, piperidyl-C(═O)O—, methylsulfonyloxy, trifluoromethylsulfonyloxy, benzenesulfonyloxy, tosyloxy, optionally halogenated C₁₋₆ alkylthio, 5- to 14-membered heteroaryl, 3- to 14-membered heterocycloalkyl, formyl, optionally halogenated C₁₋₆ alkyl-C(═O)—, C₆₋₁₄ aryl-C(═O)—, 5- to 14-membered heteroaryl-C(═O)—, 3- to 14-membered heterocycloalkyl-C(═O)—, C₁₋₆ alkoxy-C(═O)—, phenoxy-C(═O)—, 1-naphthoxy-C(═O)—, 2-naphthoxy-C(═O)—, benzyloxy-C(═O)—, phenyl-ethyl-OC(═O)—, carbamoyl, thiocarbamoyl, mono or di-C₁₋₆ alkyl-carbamoyl, phenylcarbamoyl, pyridylcarbamoyl, thiophenecarbamoyl, morpholinecarbamoyl, piperidinecarbamoyl, optionally halogenated C₁₋₆ alkylsulfonyl, phenylsulfonyl, tolylsulfonyl, 1-naphthalenesulfonyl, 2-naphthalenesulfonyl, pyridylsulfinyl, thiophenesulfinyl, optionally halogenated C₁₋₆ alkylsulfinyl, phenylsulfinyl, 1-naphthalenesulfinyl, 2-naphthalenesulfinyl, pyridinesulfinyl, thiophenesulfinyl, amino, methylamino, ethylamino, propylamino, isopropylamino, butylamino, dimethylamino, diethylamino, dipropylamino, dibutylamino, N-ethyl-N-methylamino, phenylamino, pyridylamino, benzylamino, formamido, acetamido, propionylamino, butylamino, N-acetyl-N-methylamino, phenyl-C(═O)NH¬-, naphthyl-C(═O)NH¬-, methoxy-C(═O)NH¬-, ethoxy-C(═O)NH¬-, propoxy-C(═O)NH¬-, butoxy-C(═O)NH¬-, tert-butoxy-C(═O)NH¬-, benzyloxy-C(═O)NH¬-, methylsulfonylamino, ethylsulfonylamino, benzenesulfonylamino, toluenesulfonylamino.

In one example, the group S substituents include: hydrogen, deuterium, halogen, C₁₋₆ alkyl, C₂₋₆ alkenyl, C₂₋₆ alkynyl, C₁₋₆ deuterated alkyl, C₁₋₆ haloalkyl, C₁₋₆ alkoxy, C₁₋₆ haloalkoxy, cyano-substituted C₁₋₆ alkyl, C₃₋₁₂ cycloalkyl, 3- to 14-membered heterocycloalkyl. Preferably, the group S substituents include: hydrogen, halogen, C₁₋₆ alkyl, C₁₋₆ haloalkyl, C₃₋₆ cycloalkyl. Preferably, the group S substituents include: hydrogen, fluorine, chlorine, bromine, methyl, trifluoromethyl, and cyclopropyl.

In one example, in any group, the 5- or 6-membered monocyclic heteroaryl group is selected from: thiophene, N-alkylpyrrolidone, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine , pyrazine.

In one example, in any group, the 5- or 6-membered monocyclic heteroaryl group is selected from:

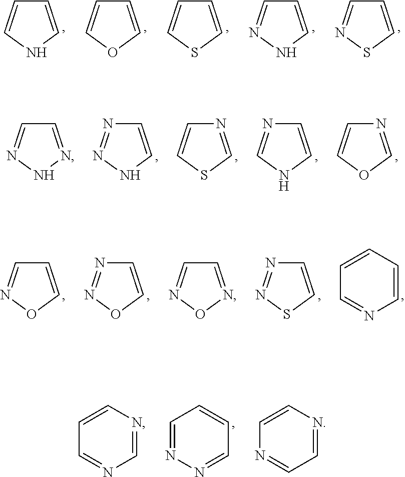

In one example, the compound of formula (I) is selected from the compounds in Table A;

TABLE A

| | |
|---|---|
| 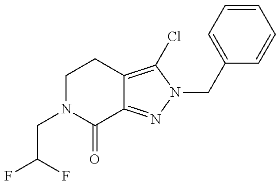 [SMILES: O=C1c2nn(Cc3ccccc3)c(Cl)c2CCN1CC(F)F] | Z1 |
| 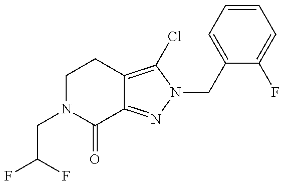 [SMILES: O=C1c2nn(Cc3ccccc3F)c(Cl)c2CCN1CC(F)F] | Z2 |
| 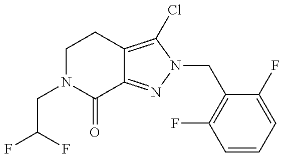 [SMILES: O=C1c2nn(Cc3c(F)cccc3F)c(Cl)c2CCN1CC(F)F] | Z3 |
| 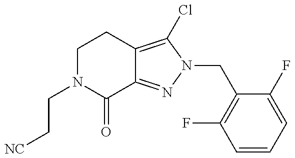 [SMILES: N#CCCN1CCc2c(nn(Cc3c(F)cccc3F)c2Cl)C1=O] | Z4 |
| 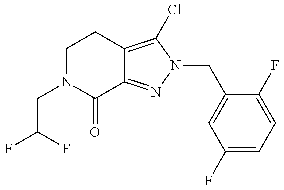 [SMILES: O=C1c2nn(Cc3cc(F)ccc3F)c(Cl)c2CCN1CC(F)F] | Z5 |
| 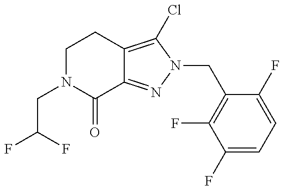 [SMILES: O=C1c2nn(Cc3c(F)ccc(F)c3F)c(Cl)c2CCN1CC(F)F] | Z6 |
| 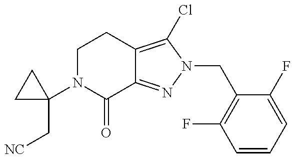 [SMILES: N#CCC1(N2CCc3c(nn(Cc4c(F)cccc4F)c3Cl)C2=O)CC1] | Z7 |
| 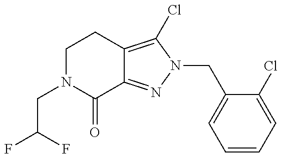 [SMILES: O=C1c2nn(Cc3ccccc3Cl)c(Cl)c2CCN1CC(F)F] | Z8 |
| 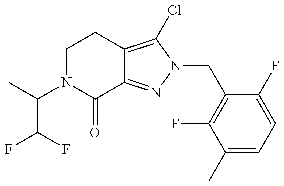 [SMILES: Cc1ccc(F)c(Cn2nc3c(c2Cl)CCN(C(C)C(F)F)C3=O)c1F] | Z9 |
| 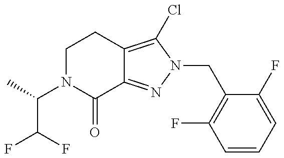 [SMILES: C[C@@H](C(F)F)N1CCc2c(nn(Cc3c(F)cccc3F)c2Cl)C1=O] | Z9-1 |
| 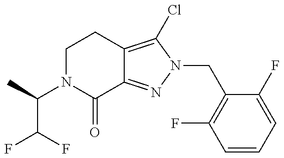 [SMILES: C[C@H](C(F)F)N1CCc2c(nn(Cc3c(F)cccc3F)c2Cl)C1=O] | Z9-2 |
| 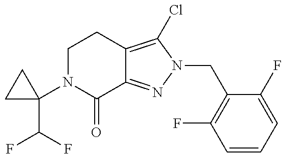 [SMILES: O=C1c2nn(Cc3c(F)cccc3F)c(Cl)c2CCN1C1(C(F)F)CC1] | Z10 |
| 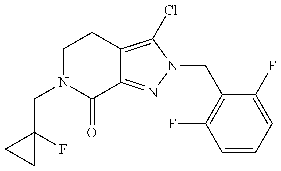 [SMILES: O=C1c2nn(Cc3c(F)cccc3F)c(Cl)c2CCN1CC1(F)CC1] | Z11 |
| 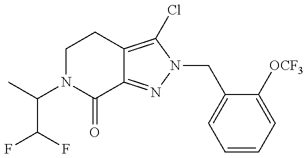 [SMILES: CC(C(F)F)N1CCc2c(nn(Cc3ccccc3OC(F)(F)F)c2Cl)C1=O] | Z12 |
| 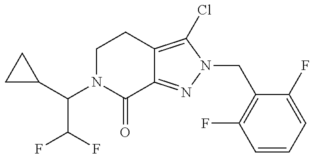 [SMILES: O=C1c2nn(Cc3c(F)cccc3F)c(Cl)c2CCN1C(C(F)F)C1CC1] | Z13 |
| 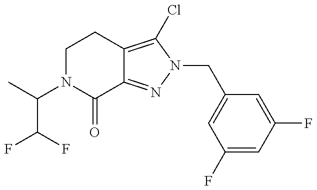 [SMILES: CC(C(F)F)N1CCc2c(nn(Cc3cc(F)cc(F)c3)c2Cl)C1=O] | Z14 |
| 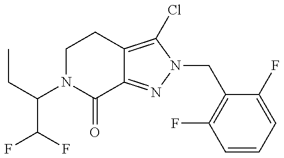 [SMILES: CCC(C(F)F)N1CCc2c(nn(Cc3c(F)cccc3F)c2Cl)C1=O] | Z15 |
| 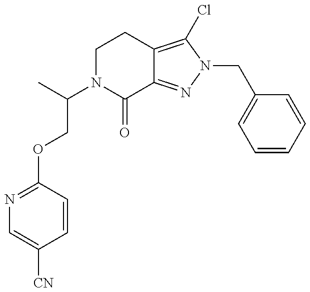 [SMILES: CC(COc1ccc(C#N)cn1)N1CCc2c(nn(Cc3ccccc3)c2Cl)C1=O] | Z16 |
| 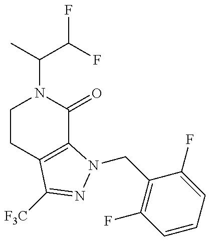 [SMILES: CC(C(F)F)N1CCc2c(C(F)(F)F)nn(Cc3c(F)cccc3F)c2C1=O] | Z17 |
| 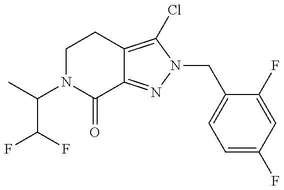 [SMILES: CC(C(F)F)N1CCc2c(nn(Cc3ccc(F)cc3F)c2Cl)C1=O] | Z18 |
| 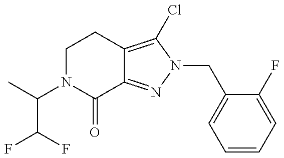 [SMILES: CC(C(F)F)N1CCc2c(nn(Cc3ccccc3F)c2Cl)C1=O] | Z19 |
| 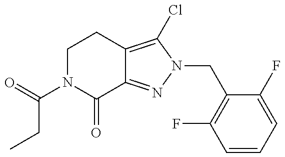 [SMILES: CCC(=O)N1CCc2c(nn(Cc3c(F)cccc3F)c2Cl)C1=O] | Z20 |
| 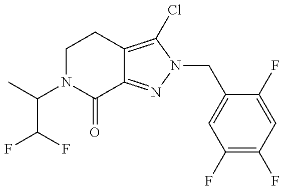 [SMILES: CC(C(F)F)N1CCc2c(nn(Cc3cc(F)c(F)cc3F)c2Cl)C1=O] | Z21 |
| 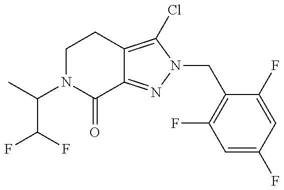 [SMILES: CC(C(F)F)N1CCc2c(nn(Cc3c(F)cc(F)cc3F)c2Cl)C1=O] | Z22 |
| 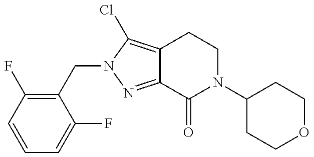 [SMILES: O=C1c2nn(Cc3c(F)cccc3F)c(Cl)c2CCN1C1CCOCC1] | Z23 |
| 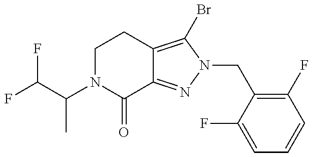 [SMILES: CC(C(F)F)N1CCc2c(nn(Cc3c(F)cccc3F)c2Br)C1=O] | Z24 |
| 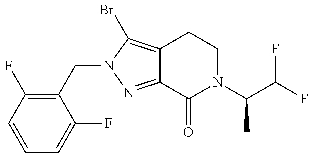 [SMILES: C[C@H](C(F)F)N1CCc2c(nn(Cc3c(F)cccc3F)c2Br)C1=O] | Z24-1 |
| 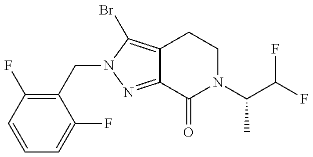 [SMILES: C[C@@H](C(F)F)N1CCc2c(nn(Cc3c(F)cccc3F)c2Br)C1=O] | Z24-2 |
| 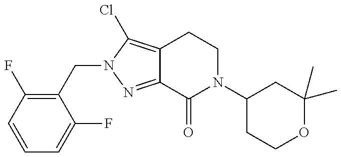 [SMILES: CC1(C)CC(N2CCc3c(nn(Cc4c(F)cccc4F)c3Cl)C2=O)CCO1] | Z25 |
| 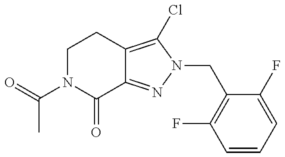 [SMILES: CC(=O)N1CCc2c(nn(Cc3c(F)cccc3F)c2Cl)C1=O] | Z26 |
| 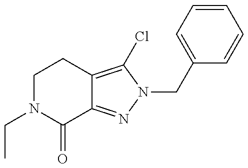 [SMILES: CCN1CCc2c(nn(Cc3ccccc3)c2Cl)C1=O] | Z27 |
| 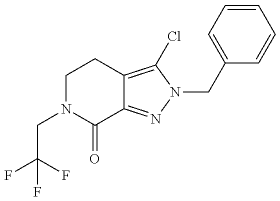 [SMILES: O=C1c2nn(Cc3ccccc3)c(Cl)c2CCN1CC(F)(F)F] | Z28 |
| 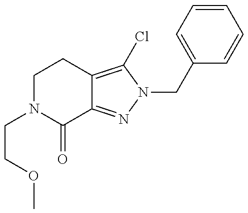 [SMILES: COCCN1CCc2c(nn(Cc3ccccc3)c2Cl)C1=O] | Z29 |
| 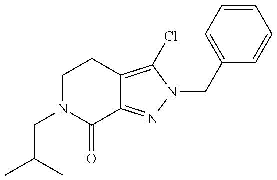 [SMILES: CC(C)CN1CCc2c(nn(Cc3ccccc3)c2Cl)C1=O] | Z30 |
| 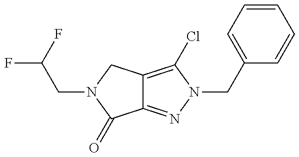 [SMILES: O=C1c2nn(Cc3ccccc3)c(Cl)c2CN1CC(F)F] | Z31 |
| 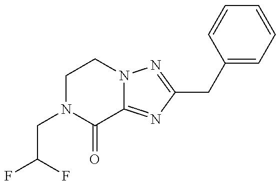 [SMILES: O=C1c2nc(Cc3ccccc3)nn2CCN1CC(F)F] | Z32 |
| 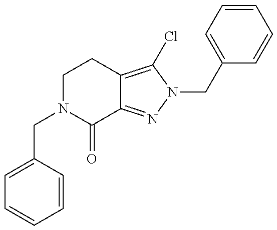 [SMILES: O=C1c2nn(Cc3ccccc3)c(Cl)c2CCN1Cc1ccccc1] | Z33 |
| 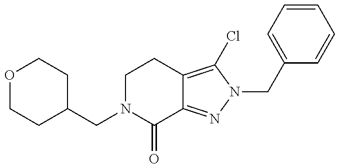 [SMILES: O=C1c2nn(Cc3ccccc3)c(Cl)c2CCN1CC1CCOCC1] | Z34 |
| 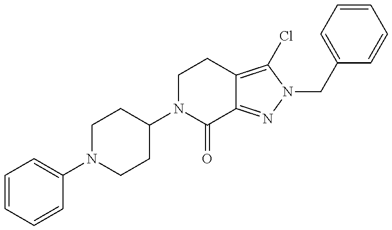 [SMILES: O=C1c2nn(Cc3ccccc3)c(Cl)c2CCN1C1CCN(c2ccccc2)CC1] | Z35 |
| 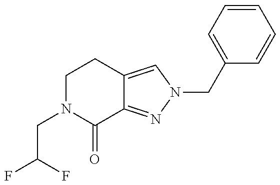 [SMILES: O=C1c2nn(Cc3ccccc3)cc2CCN1CC(F)F] | Z36 |
| 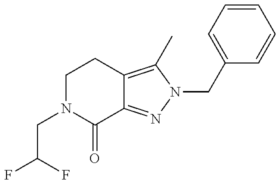 [SMILES: Cc1c2c(nn1Cc1ccccc1)C(=O)N(CC(F)F)CC2] | Z37 |
| 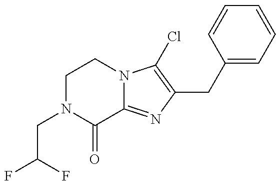 [SMILES: O=C1c2nc(Cc3ccccc3)c(Cl)n2CCN1CC(F)F] | Z38 |
| 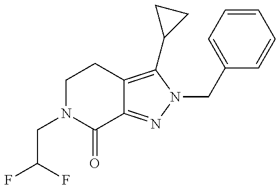 [SMILES: O=C1c2nn(Cc3ccccc3)c(C3CC3)c2CCN1CC(F)F] | Z39 |
| 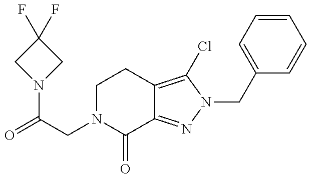 [SMILES: O=C(CN1CCc2c(nn(Cc3ccccc3)c2Cl)C1=O)N1CC(F)(F)C1] | Z40 |
| 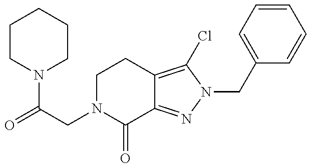 [SMILES: O=C(CN1CCc2c(nn(Cc3ccccc3)c2Cl)C1=O)N1CCCCC1] | Z41 |
| 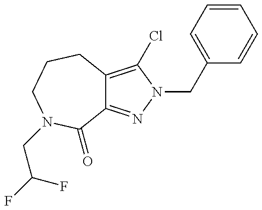 [SMILES: O=C1c2nn(Cc3ccccc3)c(Cl)c2CCCN1CC(F)F] | Z42 |
| 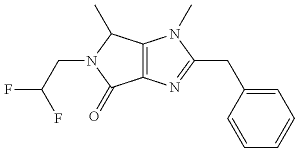 [SMILES: CC1c2c(nc(Cc3ccccc3)n2C)C(=O)N1CC(F)F] | Z43 |
| 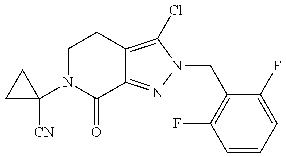 [SMILES: N#CC1(N2CCc3c(nn(Cc4c(F)cccc4F)c3Cl)C2=O)CC1] | Z44 |
| 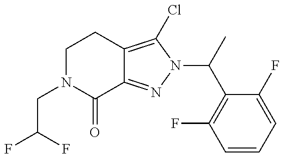 [SMILES: CC(c1c(F)cccc1F)n1nc2c(c1Cl)CCN(CC(F)F)C2=O] | Z45 |
| 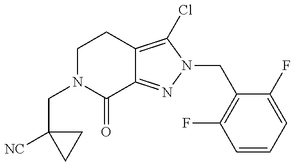 [SMILES: N#CC1(CN2CCc3c(nn(Cc4c(F)cccc4F)c3Cl)C2=O)CC1] | Z46 |
| 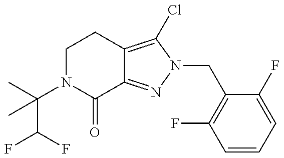 [SMILES: CC(C)(C(F)F)N1CCc2c(nn(Cc3c(F)cccc3F)c2Cl)C1=O] | Z47 |
| 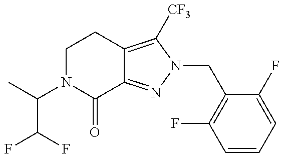 [SMILES: CC(C(F)F)N1CCc2c(nn(Cc3c(F)cccc3F)c2C(F)(F)F)C1=O] | Z48 |
| 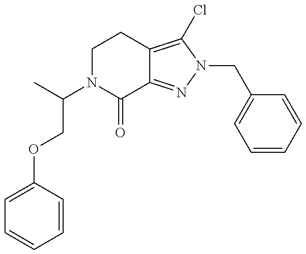 [SMILES: CC(COc1ccccc1)N1CCc2c(nn(Cc3ccccc3)c2Cl)C1=O] | Z49 |
| 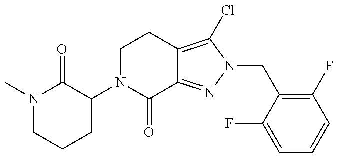 [SMILES: CN1CCCC(N2CCc3c(nn(Cc4c(F)cccc4F)c3Cl)C2=O)C1=O] | Z50 |

In one example, the compound of formula (I) is selected from the compounds prepared in the examples of the present application. For example, it is selected from compounds Z1 to Z26.

In the second aspect, the present invention provides a pharmaceutical composition comprising the compound of the first aspect of the present invention or a pharmaceutically acceptable salt, stereoisomer, solvate or prodrug thereof; and pharmaceutically acceptable carrier.

In the third aspect, the present invention provides the use of the compound of the first aspect of the present invention or a pharmaceutically acceptable salt, stereoisomer, solvate or prodrug thereof or the pharmaceutical composition of the second aspect of the present invention in preparing medicament to treat and/or prevent disease.

In one example, the disease is selected from the group consisting of stroke, inflammatory bowel disease, ulcerative colitis, Crohn's disease, psoriasis, rheumatoid arthritis, NASH and heart failure.

In the fourth aspect, the present invention provides the use of the compound of the first aspect of the present invention or a pharmaceutically acceptable salt, stereoisomer, solvate or prodrug thereof or the pharmaceutical composition of the second aspect of the present invention in preparing RIPK1 selective inhibitors, the RIPK1 selective inhibitors is used in treating RIPK1 related diseases or conditions.

In one example, the RIPK1-related diseases or conditions include, but are not limited to inflammatory diseases, for example Crohn's disease and ulcerative colitis, inflammatory bowel disease, asthma, graft versus host disease, and chronic obstructive pulmonary disease; autoimmune diseases, such as Graves' disease, rheumatoid arthritis, systemic lupus erythematosus, psoriasis; destructive bone diseases, such as bone resorption diseases, osteoarthritis, osteoporosis, multiple myeloma-related bone disease diseases; proliferative diseases, such as acute myeloid leukemia, chronic myelogenous leukemia; angiogenesis disorders, such as angiogenesis disorders, including solid tumors, ocular neovascularization and infantile hemangioma; infectious diseases, such as sepsis, septic shock and Hirsch disease; neurodegenerative diseases such as Alzheimer's disease, Parkinson's disease, amyotrophic lateral sclerosis, cerebral ischemia or neurodegenerative diseases caused by traumatic injury, tumors and viral diseases such as metastatic melanoma, Kaposi's sarcoma, multiple myeloma, HIV infection and CMV retinitis and AIDS.

In one example, the RIPK1-related diseases or conditions include, but are not limited to pancreatitis (acute or chronic), asthma, allergies, adult respiratory distress syndrome, chronic obstructive pulmonary disease, glomerulonephritis, rheumatoid arthritis, systemic lupus erythematosus, scleroderma, chronic thyroiditis, Graves disease, autoimmune gastritis, diabetes, autoimmune hemolytic anemia, autoimmune neutropenia, thrombocytopenia, atopic dermatitis, chronic active hepatitis, myasthenia gravis, amyotrophic lateral sclerosis disease, multiple sclerosis, inflammatory bowel disease, ulcerative colitis, Crohn's disease, psoriasis, graft versus host disease, endotoxin-induced inflammation, tuberculosis, atherosclerosis, muscle degeneration, cachexia, silver Dermatitis arthritis, Rett syndrome, gout, traumatic arthritis, rubella arthritis, acute synovitis, pancreatic beta-cell disease; diseases characterized by massive neutrophil infiltration; rheumatoid spondylitis, gout arthritis and other arthritic conditions, cerebral malaria, chronic pulmonary inflammation, silicosis, pulmonary sarcoidosis, bone resorptive diseases, allograft rejection, fever and myalgia due to infection, cachexia secondary to infection, luteoid formation, scar tissue formation, ulcerative colitis, fever, influenza, osteoporosis, osteoarthritis, acute myeloid leukemia, chronic myelogenous leukemia, metastatic melanoma, Kaposi's sarcoma, multiple myeloid tumors, sepsis, septic shock, and shigellosis; Alzheimer's disease, Parkinson's disease, neurodegenerative diseases caused by cerebral ischemia, or traumatic injury; angiogenic disorders including solid tumors, ocular neovascularization, and infants Hemangioma; viral diseases including acute hepatitis infection (including hepatitis A, hepatitis B, and hepatitis C), HIV infection and CMV retinitis, AIDS, ARC or malignancy and herpes; stroke, myocardial ischemia, cardiac stroke illness, organ ischemia, vascular proliferation, cardiac and renal reperfusion injury, thrombosis, cardiac hypertrophy, thrombin-induced platelet aggregation, endotoxemia and/or toxic shock syndrome, and prostaglandin endoperoxides enzyme synthase-2-related disorders and pemphigus vulgaris.

In one example, the RIPK1-related diseases or conditions are selected from stroke, inflammatory bowel disease, Crohn's disease and ulcerative colitis, allograft rejection, rheumatoid arthritis, psoriasis, ankylosing spondylitis, psoriatic arthritis and pemphigus vulgaris. Alternatively, the preferred condition is selected from ischemia-reperfusion injury, including cerebral ischemia-reperfusion injury caused by stroke and myocardial ischemia-reperfusion injury caused by myocardial infarction.

In the fifth aspect, the present invention provides a preparation method of the compound of the first aspect of the invention or a pharmaceutically acceptable salt, stereoisomer, solvate or prodrug thereof, wherein the preparation method includes steps selected from the following schemes:

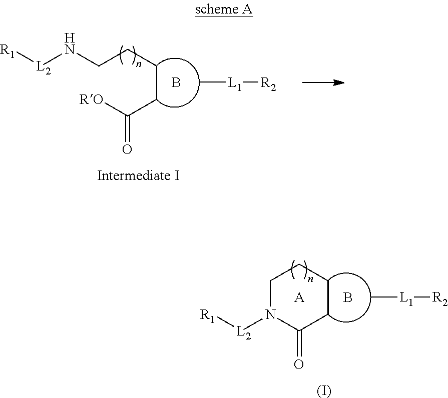

(1) prepare intermediate I with ring B;
(2) the intermediate I forms ring A through a ring-closing reaction, thereby obtaining the compound as formula (I); wherein, R′ is selected from hydrogen or C₁₋₃ alkyl: or

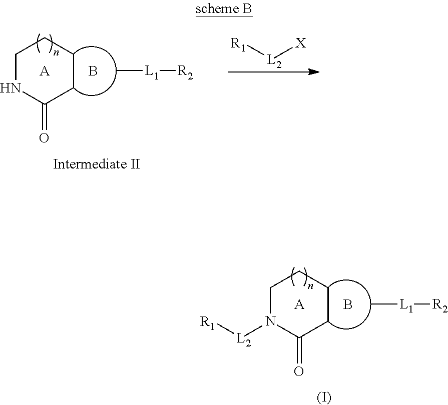

(1) prepare intermediate II;
(2) the intermediate II reacts with the corresponding R₁-L₂-X, thus obtain the compound represented by formula (I), wherein X represents halogen, preferably, X is selected from F, Cl, Br, I, preferably, X is Cl. It should be understood that within the scope of the present invention, the above-mentioned technical features of the present invention and the technical features specifically described below (such as examples) can be combined with each other to form new or preferred technical solutions. Due to space limitations, they will not be described one by one here.

### Definitions of Terms

As used herein, the term “heteroatom” is selected from nitrogen, oxygen or sulfur. Wherein, the nitrogen can be optionally substituted; the sulfur can also be optionally substituted, for example oxo, which forms S(O)ₜ₃ (wherein t3 is an integer from 0 to 2).

As used herein, when a group such as an alkyl group is located in the middle of a structural formula, the group is a subunit, For example, an alkyl group is an alkylene group, etc.

As used herein, the term “alkyl” refers to a chain (straight or branched) saturated aliphatic hydrocarbon group. The term “alkyl” may be a straight or branched alkyl group containing 1 to 20 carbon atoms (C₁₋₂₀ alkyl), preferably an alkyl group containing 1 to 12 carbon atoms (C₁₋₁₂ alkyl), more preferably a lower alkyl group (C₁₋₆ alkyl) containing 1 to 6 carbon atoms, and non-limiting examples include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. A lower alkyl group (C₁₋₃ alkyl) containing 1 to 3 carbon atoms is more preferred, and non-limiting examples thereof include methyl, ethyl, n-propyl, isopropyl, etc. The alkyl group may be substituted or unsubstituted. When the alkyl group is substituted, the substituent is preferably one or more groups described in the present application.

As used herein, the terms “cycloalkyl” and “cycloalkyl ring” are used interchangeably and referred to saturated or partially unsaturated monocyclic or polycyclic cyclic hydrocarbon group. The term “cycloalkyl” may be a cycloalkyl containing 3 to 12 carbon atoms (C₃₋₁₂ cycloalkyl), more preferably a cycloalkyl containing 3 to 10 carbon atoms (C₃₋₁₀ cycloalkyl), more preferably a cycloalkyl group containing 3 to 6 carbon atoms (C₃₋₆ cycloalkyl). The ring carbon atoms of the cycloalkyl group may be optionally substituted by 1, 2 or 3 oxo group(s) to form a cyclic ketone structure. When the cycloalkyl is a monocyclic cycloalkyl, preferably a monocyclic cycloalkyl containing 3 to 8 ring carbon atoms (i.e., 3- to 8-membered or C₃₋₈). Herein, “C3-8 monocyclic cycloalkyl” and “C₃₋₈ cycloalkyl” can be used interchangeably, more preferably a monocyclic cycloalkyl containing 3 to 6 ring carbon atoms (i.e., C₃₋₆), the non-limiting examples of monocyclic cycloalkyl (or C₃₋₆ cycloalkyl) include cyclopropyl, cyclobutyl, cyclopentyl, cyclopentenyl, cyclohexyl, cyclohexenyl, cyclohexadienyl, cycloheptyl, cycloheptatrienyl, cyclooctyl, cyclobutanone, cyclohexenyl butane-1,2-dione, cyclopentanone, cyclopentane-1,3-dione, cyclohexanone, cyclohexane-1,3-dione, etc.

When the cycloalkyl is a polycyclic cycloalkyl, the polycyclic cycloalkyl group includes spirocycloalkyl, fused cycloalkyl and bridged cycloalkyl.

As used herein, the term “spirocycloalkyl” refers to a saturated or partially unsaturated polycyclic cyclic hydrocarbon group wherein the rings in the system share one carbon atom (referred to as spiro atom). The term “saturated spirocycloalkyl” refers that there are no unsaturated bonds in the spirocycloalkyl. The term “partially unsaturated spirocycloalkyl” refers to each single ring in a spirocycloalkyl group may contain one or more double bonds, but no ring has a fully conjugated x electron system. The term “spirocycloalkyl” can be a spirocycloalkyl group containing 5 to 12 ring carbon atoms (C₅₋₁₂), wherein one carbon atom (referred to as spiro atom) is shared between the single rings. It is preferably a 6- to 12-membered spirocycloalkyl group, and more preferably a 7- to 11-membered spirocycloalkyl group. According to the number of spiro atoms shared between the rings, spirocycloalkyl groups are divided into monospirocycloalkyl groups, bispirocycloalkyl groups or polyspirocycloalkyl groups, preferably monospirocycloalkyl groups, more preferably 7-membered (4-membered monocyclic ring/4-membered monocyclic ring), 8-membered (4-membered monocyclic ring/5-membered monocyclic ring), 9-membered (4-membered monocyclic ring/6-membered monocyclic ring, 5-membered monocyclic ring/5-membered monocyclic ring), 10 -membered (5-membered monocyclic ring/6-membered monocyclic ring) or 11-membered (6-membered monocyclic ring/6-membered monocyclic ring) monospirocycloalkyl groups. Non-limiting examples of spirocycloalkyl (or 7- to 11-membered spirocycloalkyl) include:

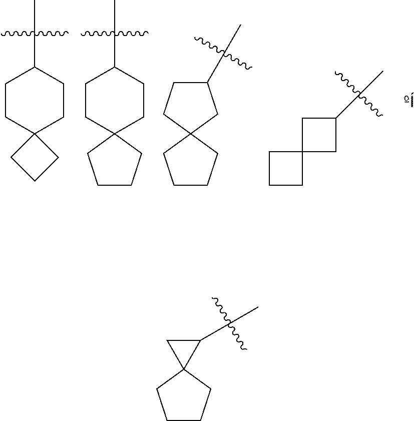

As used herein, the term “fused cycloalkyl” refers to a saturated or partially unsaturated polycyclic cyclic hydrocarbon group wherein each ring in the system shares a pair of adjacent carbon atoms with every other ring in the system. The term “saturated fused cycloalkyl” refers that there are no unsaturated bonds in the fused cycloalkyl. The term “partially unsaturated fused cycloalkyl” refers to one or more rings in a fused cycloalkyl group may contain one or more double bonds, but no ring has a fully conjugated « electron system. The term “fused cycloalkyl” can be a fused cycloalkyl group containing 5 to 12 ring carbon atoms (i.e., C₅₋₁₂). It is preferably a 6- to 12-membered fused ring alkyl group, and more preferably a 6- to 10-membered fused ring alkyl group. According to the number of rings formed, it can be divided into bicyclic, tricyclic, tetracyclic or polycyclic fused ring alkyl groups, preferably bicyclic, more preferably 8-membered (5-membered monocyclic ring fused with 5-membered monocyclic ring), 9-membered (5-membered monocyclic ring fused with 6-membered monocyclic ring) or 10-membered (6-membered monocyclic ring and 6-membered monocyclic ring fused) bicyclic fused ring alkyl. Non-limiting examples of fused ring alkyl groups (or 6- to 10-membered fused ring alkyl groups) include:

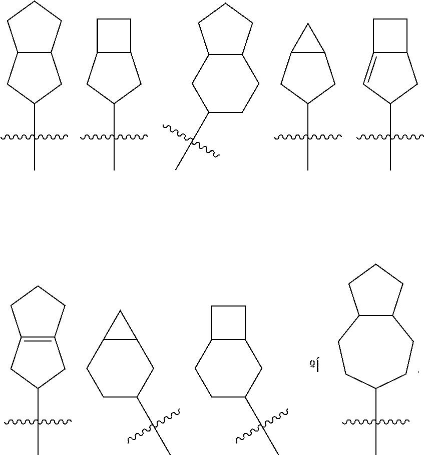

As used herein, the term “bridged cycloalkyl” refers to a saturated or partially unsaturated polycyclic cyclic hydrocarbon group wherein any two rings in the system share two carbon atoms that are not directly connected. The term “saturated bridged cycloalkyl” refers that there are no unsaturated bonds in the bridged cycloalkyl. The term “partially unsaturated bridged cycloalkyl” refers to a bridged cycloalkyl group that has one or more double bonds but none of the rings has a fully conjugated x electron system. The term “bridged cycloalkyl” may be a bridged cycloalkyl group containing 5 to 12 ring carbon atoms (i.e., C₅₋₁₂). It is preferably a 6- to 12-membered bridged cycloalkyl group, and more preferably a 7- to 10-membered bridged cycloalkyl group. According to the number of constituent rings, it can be divided into bicyclic, tricyclic, tetracyclic or polycyclic bridged cycloalkyl groups, preferably bicyclic. Non-limiting examples of bridged cycloalkyl groups include:

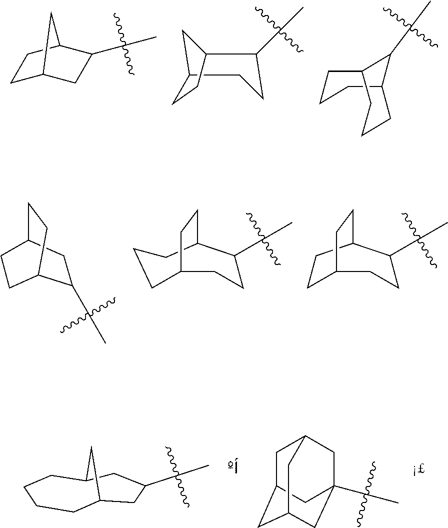

The cycloalkyl ring can be fused to an aryl, heteroaryl or heterocycloalkyl ring, wherein the ring attached to the parent structure is a cycloalkyl ring, non-limiting examples thereof include indanyl, tetrahydronaphthyl, benzocycloheptyl, etc. The cycloalkyl group may be optionally substituted or unsubstituted. When substituted, the substituent is preferably one or more groups described in the present application.

As used herein, the term “C₂₋₆ alkenyl” refers to an alkyl group as defined above consisting of 2 to 6 carbon atoms and at least one carbon-carbon double bond, more preferably C₂₋₄ alkenyl group consisting of 2 to 4 carbon atoms and 1 to 2 carbon-carbon double bond(s), for example vinyl, 1-propenyl, 2-propenyl, 1-, 2- or 3-butenyl, etc. The alkenyl group may be substituted or unsubstituted. When substituted, the substituent is preferably one or more groups described in the present application.

As used herein, the term “C₂₋₆ alkynyl” refers to an alkyl group as defined above consisting of 2 to 6 carbon atoms and at least one carbon-carbon triple bond, more preferably C₂₋₄ alkynyl group consisting of 2 to 4 carbon atoms and 1 to 2 carbon-carbon triple bond(s), for example ethynyl, 1-propynyl, 2-propynyl, 1-, 2- or 3-butynyl, etc. The alkynyl group may be substituted or unsubstituted. When substituted, the substituent is preferably one or more groups described in the present application.

As used herein, the terms “heterocycloalkyl” and “heterocycloalkyl ring” are used interchangeably and refer to saturated or partially unsaturated monocyclic or polycyclic cyclic hydrocarbon groups wherein one or more (preferably 1 to 4 or 1 to 3 or 1 to 2) ring atoms are heteroatoms selected from nitrogen, oxygen or S(O)ₜ₃ (wherein t3 is an integer from 0 to 2), but does not include the ring part of —O—O—, —O—S— or —S—S—, the remaining ring atoms are carbon. The term “heterocycloalkyl” may be a heterocycloalkyl group containing 3 to 14 ring atoms (i.e., 3- to 14-membered); preferably a 3- to 12-membered heterocycloalkyl group; more preferably a 3- to 10-membered heterocycloalkyl group, more preferably a 3- to 6-membered heterocycloalkyl; wherein one or more (preferably 1 to 4) ring atoms are heteroatoms selected from nitrogen, oxygen or S(O)ₜ₃ (wherein t3 is an integer from 0 to 2), but does not include the ring part of —O—O—, —O—S— or —S—S—, and the remaining ring atoms are carbon. The nitrogen atom may be substituted or unsubstituted (i.e., N or NR, R is hydrogen or any of the substituents already defined herein). The ring carbon atoms of the heterocycloalkyl group may be optionally substituted by 1, 2 or 3 oxo group(s) to form a cyclic ketone, cyclic lactone or cyclic lactam structure.

As used herein, in “3- to 14-membered heterocycloalkyl”, “3- to 12-membered heterocycloalkyl”, “3- to 10-membered heterocycloalkyl” or “3- to 6-membered heterocycloalkyl”, when these heterocycloalkyl groups are 3-membered heterocycloalkyl groups and contain only one heteroatom as a ring atom, the heteroatom is not a nitrogen atom.

When the heterocycloalkyl group is a monocyclic heterocycloalkyl group, the monocyclic heterocycloalkyl group is saturated or partially unsaturated, preferably monocyclic heterocycloalkyl containing 3 to 8 ring atoms (i.e., 3- to 8-membered) wherein 1, 2 or 3 ring atom(s) are heteroatom(s). More preferred are monocyclic heterocycloalkyl groups containing 3 to 6 ring atoms (i.e., 3- to 6-membered), wherein 1, 2 or 3 ring atom(s) are heteroatom(s). Most preferred are monocyclic heterocycloalkyl groups containing 5 or 6 ring atoms (i.e., 5- or 6-membered), wherein 1, 2 or 3 ring atom(s) are heteroatom(s). When the heteroatom is a nitrogen atom, the nitrogen atom may be substituted or unsubstituted (i.e., N or NR, R is hydrogen or other substituents already defined herein). When the heteroatom is a sulfur atom, the sulfur atom may be optionally oxidized (i.e., S(O)ₜ₃, t3 is an integer from 0 to 2). The ring carbon atoms of the monocyclic heterocycloalkyl group may be optionally substituted by 1, 2 or 3 oxo group(s) to form a cyclic ketone, cyclic lactone or cyclic lactam structure. Non-limiting examples of monocyclic heterocycloalkyl groups include: aziradine, ethylene oxide, azetidine, azetidin-2-one, oxetane, oxetan-2-one, oxazolidine, pyrrolidine-2-one, pyrrolidine-2,5-dione, 1,3-dioxolane, dihydrofuran-2(3H)-one, dihydrofuran-2,5-dione, piperidin-2-one, piperidine-2,6-dione, tetrahydro-2H-pyran-2-one, imidazolidine, tetrahydrofuran, tetrahydrothiophene, tetrahydropyrrole, 1,3-dioxolane-2-one, oxazolidin-2-one, imidazolidine-2-one, piperidine, piperazine, piperazin-2-one, morpholine, morpholin-3-one, morpholin-2-one, thiomorpholin-3-one 1,1-dioxide, thiomorpholine , thiomorpholine-1,1-dioxide, tetrahydropyran, 1,2-dihydroazacyclobutadiene, 1,2-dihydrooxetadiene, 2,5-dihydro-1H-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, 1,3-oxazine, hexahydropyrimidine, 1,4-dioxane, ectoine-2(1H)-one, 1,4-dioxan-2-one, 5,6-dihydro-2H-pyran-2-one, 5,6-dihydropyrimidine-4(3H)-one, 3,4-dihydropyridin-2(1H)-one, 5,6-dihydropyridin-2(1H)-one, 5,6-dihydropyrimidine-4(1H)-one, pyrimidine-4(3H)-one, pyrimidine-4(1H)-one, 4,5-dihydro-1H-imidazole, 2,3-dihydro-1H-imidazole, 2,3-dihydroxazole, 1,3-dioxole, 2,3-dihydrothiophene, 2,5-dihydrothiophene, 3,4-dihydro-2H-1,4-oxazine, 3,4-dihydro-2H-1,4-thiazine 1,1-dioxide, 1,2,3,4-tetrahydropyrazine, 1,3-dihydro-2H-pyrrole-2-one, 1,5-dihydro-2H-pyrrole-2-one, 1H-pyrrole-2,5-dione, furan-2(3H)-one, furan-2(5H)-one, 1,3-dioxol-2-one, oxazol-2(3H)-one, 1,3-dihydro-2H-imidazol-2-one, furan-2,5-dione, 3,6-dihydropyridin-2(1H)-one, pyridine-2,6-(1H,3H)-dione, 5,6-dihydro-2H-pyran-2-one, 3,6-dihydro-2H-pyran-2-one, 3,4-dihydro-2H-1,3-oxazine, 3,6-dihydro-2H-1,3-oxazine, 1,2,3,4-tetrahydropyrimidine, etc.

As used herein, “3- to 6-membered monoheterocycle” or “3- to 6-membered monocyclic heterocycloalkyl” are used interchangeably and refer to 3- to 6-membered saturated or partially unsaturated monocyclic rings wherein 1, 2 or 3 carbon atoms are replaced by heteroatoms selected from nitrogen, oxygen or S(O)ₜ₅ (where t5 is an integer from 0 to 2), but does not include the ring part of —O—O—, —O—S— or —S—S—, and the rest ring atoms are carbon; preferably 4- to 6-membered, more preferably 5- to 6-membered. The ring carbon atoms of the monoheterocyclic ring may be optionally substituted by 1, 2 or 3 oxo group(s) to form a cyclic ketone, cyclic lactone or cyclic lactam structure. Examples of 3- to 6-membered monoheterocycles include (but are not limited to) aziridine, ethylene oxide, azetidine, oxetane, tetrahydrofuran, tetrahydrothiophene, tetrahydropyrrole, piperidine, pyrroline, oxazolidine, piperazine, dioxolane, dioxane, morpholine, thiomorpholine, thiomorpholine-1,1-dioxide, tetrahydropyran, 1,2-dihydroazacyclobutadiene, 1,2-dihydrooxetadiene, 2,5-dihydro-1H-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, etc. Non-limiting examples of 3- to 6-membered monocyclic heterocycloalkyl or 4- to 6-membered monocyclic heterocycloalkyl include:

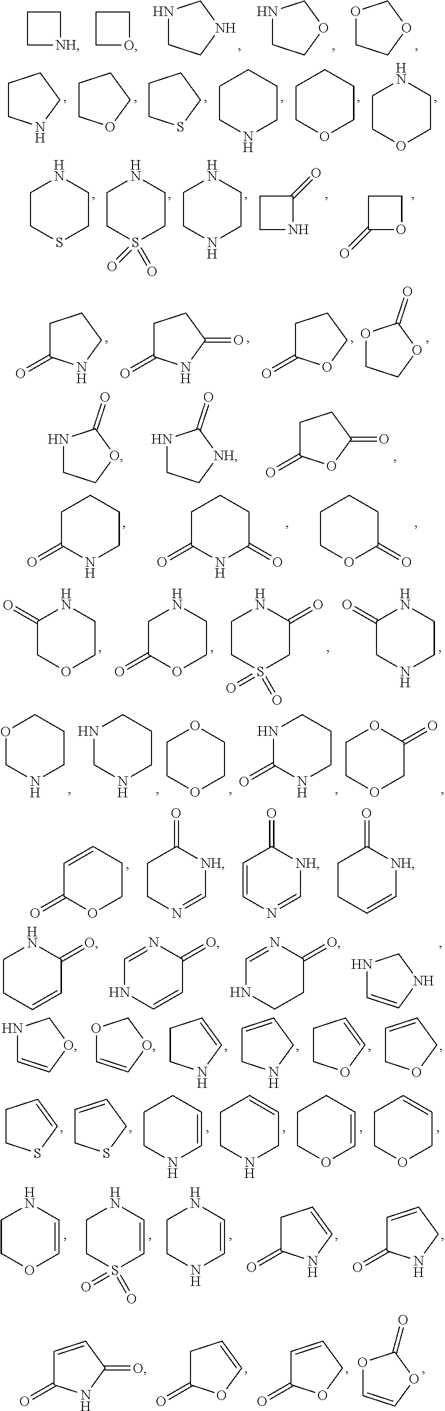

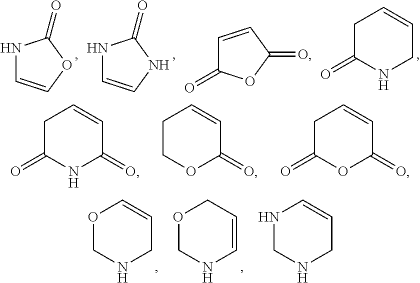

The two ring atoms connected to the above monocyclic heterocycloalkyl group, including C—C and N-C, can optionally be fused with the cycloalkyl, heterocycloalkyl, aryl or heteroaryl, such as monocyclic cycloalkyl ring, monocyclic heterocycloalkyl ring, monoaryl ring, 5- or 6-membered monocyclic heteroaryl ring, etc. defined in the present invention, to form fused polycyclic rings. The two ring atoms connected to the monocyclic heterocycloalkyl group forming a fused ring with other rings are preferably C—C.

As used herein, “3- to 6-membered nitrogen-containing heterocycloalkyl” means that one ring atom in the 3- to 6-membered heterocycloalkyl must be a nitrogen atom, all the remaining ring atoms are carbon atoms or 0, 1 or 2 ring atoms among the remaining ring atoms are each independently heteroatoms selected from nitrogen, oxygen or sulfur. When the heterocycloalkyl group is a polycyclic heterocycloalkyl group, the heterocycloalkyl group includes spiroheterocycloalkyl, fused heterocycloalkyl and bridged heterocycloalkyl.

As used herein, the term “spiroheterocycloalkyl” refers to a saturated or partially unsaturated polycyclic heterocycloalkyl group wherein the single rings share one atom (called a spiro atom), wherein one or more (e.g., 1 to 4 or 1 to 3 or 1 to 2) ring atoms are heteroatoms selected from nitrogen, oxygen, or S(O)ₜ₄ (where t4 is an integer from 0 to 2), the remaining ring atoms are carbon. The term “saturated spiroheterocycloalkyl” means that the spiroheterocycloalkyl system does not have any unsaturated bonds. The term “partially unsaturated spiroheterocycloalkyl” means that one or more rings in the spiroheterocycloalkyl system may contain one or more double bonds, but no ring has a fully conjugated π electron system. The term “spiroheterocycloalkyl” can be a spiroheterocycloalkyl group containing 5 to 14 ring atoms (i.e., 5- to 14-membered), wherein one of the 3- to 8-membered (i.e., 3 to 8 ring atoms) monocyclic rings share one atom (called a spiro atom), preferably a 6- to 14-membered spiroheterocycloalkyl group, and more preferably a 7- to 11-membered spiroheterocycloalkyl group; one or more of the ring atoms are heteroatoms selected from nitrogen, oxygen or S(O)t4 (where t4 is an integer from 0 to 2), and the remaining ring atoms are carbon. When the heteroatom is a nitrogen atom, the nitrogen atom may be substituted or unsubstituted (i.e., N or NR, R is hydrogen or other substituents already defined herein). Each single ring may contain one or more double bonds, but no ring has a fully conjugated π electron system.

According to the number of shared spiro atoms between rings, spiroheterocycloalkyl groups are divided into monospiroheterocycloalkyl, bispiroheterocycloalkyl or polyspiroheterocycloalkyl, and are preferably single spiroheterocycloalkyl and bispiroheterocycloalkyl. More preferably, they are 7-membered (4-membered monocyclic ring/4-membered monocyclic ring), 8-membered (4-membered monocyclic ring/5-membered monocyclic ring), 9-membered (4-membered monocyclic ring/6-membered monocyclic ring, 5-membered monocyclic ring/5-membered monocyclic ring), 10-membered (5-membered monocyclic ring/6-membered monocyclic ring) or 11-membered (6-membered monocyclic ring/6-membered monocyclic ring) monospiroheterocycloalkyl group. Non-limiting examples of spiroheterocycloalkyl include:

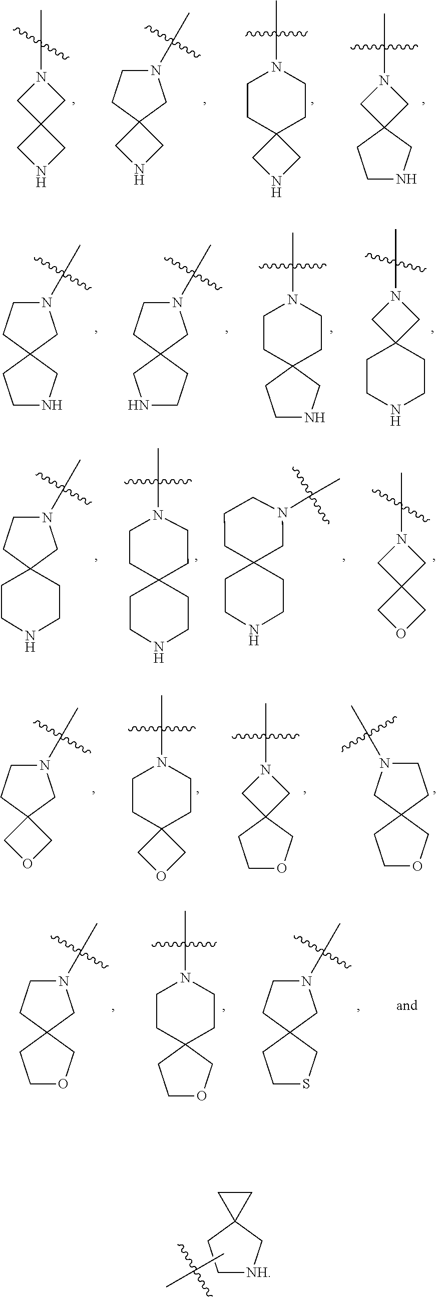

As used herein, the term “fused heterocycloalkyl” refers to a saturated or partially unsaturated polycyclic heterocycloalkyl group wherein each ring in the system shares an adjacent pair of atoms with every other ring in the system, and one or more (for example, 1 to 4 or 1 to 3 or 1 to 2) ring atoms in the system are heteroatoms selected from nitrogen, oxygen or S(O)ₜ₄ (wherein t4 is an integer from 0 to 2), the remaining ring atoms are carbon. The term “saturated fused heterocycloalkyl” refers that the fused heterocycloalkyl system does not have any unsaturated bonds. The term “partially unsaturated fused heterocycloalkyl” refers that one or more rings in the fused heterocycloalkyl system may contain one or more double bonds, but no ring has a fully conjugated π electron system. The term “fused heterocycloalkyl” may be a fused heterocycloalkyl group containing 5 to 14 ring atoms (i.e., 5- to 14-membered), preferably a 6- to 14-membered fused heterocycloalkyl group, more preferably, a 6- to 10-membered fused heterocycloalkyl group, and more preferably a 8- to 10-membered fused heterocycloalkyl group; one or more ring atoms in the system are selected from nitrogen, oxygen or S(O)ₜ₄ (wherein t4 is an integer from 0 to 2), and the remaining ring atoms are carbon. When the heteroatom is a nitrogen atom, the nitrogen atom may be substituted or unsubstituted (i.e., N or NR, R is hydrogen or other substituents already defined herein). According to the number of constituent rings, it can be divided into bicyclic, tricyclic, tetracyclic or polycyclic fused heterocycloalkyl groups, preferably bicyclic or tricyclic, more preferably 8-membered (5-membered monocyclic ring fused with 5-membered monocyclic ring), 9-membered (5-membered monocyclic ring fused with 6-membered monocyclic ring) or 10-membered (6-membered monocyclic ring fused with 6-membered monocyclic ring) bicyclic fused heterocycloalkyl group. Non-limiting examples of fused heterocycloalkyl groups include:

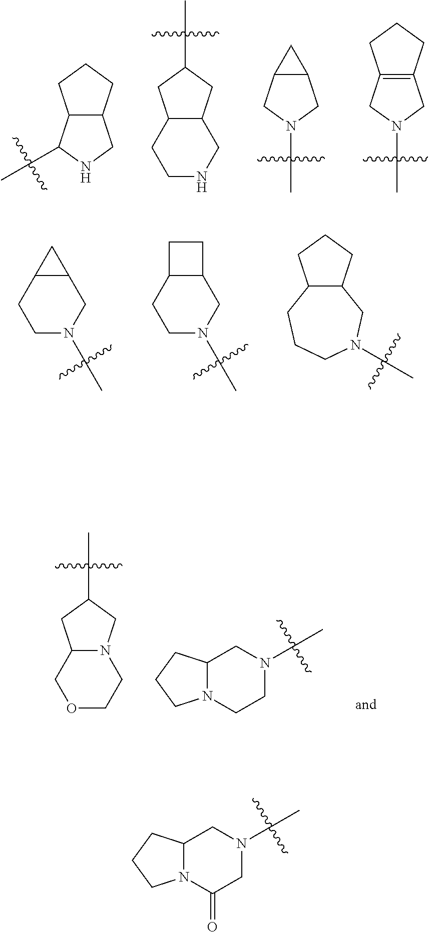

As used herein, the term “bridged heterocycloalkyl” refers to a saturated or partially unsaturated polycyclic heterocycloalkyl group wherein any two rings in the system share two atoms that are indirectly connected, wherein one or more (e.g., 1 to 4 or 1 to 3 or 1 to 2) ring atoms are heteroatoms selected from nitrogen, oxygen, or S(O)ₜ₃ (where t3 is an integer from 0 to 2), and the remaining ring atoms are carbon. The term “saturated bridged heterocycloalkyl” means that the bridged heterocycloalkyl system does not have any unsaturated bonds. The term “partially unsaturated bridged heterocycloalkyl” means that one or more rings in the bridged heterocycloalkyl system may contain one or more double bonds, but no ring has a fully conjugated x electron system. The term “bridged heterocycloalkyl” may be a bridged heterocycloalkyl group containing 5 to 14 ring atoms (i.e., 5- to 14-membered), preferably a 6- to 14-membered bridged heterocycloalkyl group, more preferably, a 7- to 10-membered bridged heterocycloalkyl group; wherein one or more (for example, 1 to 4 or 1 to 3 or 1 to 2) ring atoms are selected from nitrogen, oxygen or S(O)ₜ₃ (wherein t3 is an integer from 0 to 2), and the remaining ring atoms are carbon. According to the number of constituent rings, it can be divided into bicyclic, tricyclic, tetracyclic or polycyclic bridged heterocycloalkyl groups, preferably bicyclic, tricyclic or tetracyclic, more preferably bicyclic or tricyclic. Non-limiting examples of bridged heterocycloalkyl groups include:

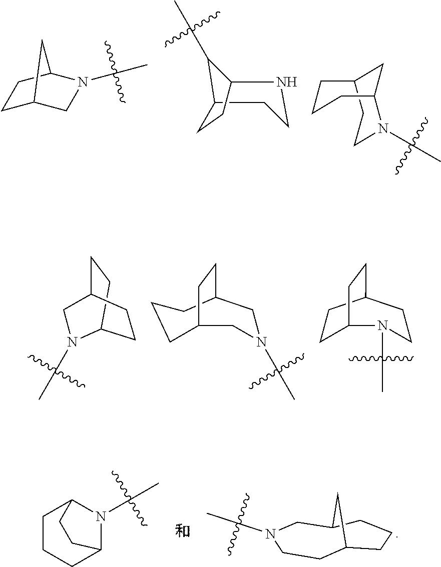

In the present invention, the above types of heterocycloalkyl groups may be optionally substituted or unsubstituted. When substituted, the substituent is preferably one or more groups described in this application.

As used herein, in the “spiroheterocycloalkyl”, “bridged heterocycloalkyl” or “fused heterocycloalkyl”, when the ring containing heteroatoms is a 3-membered ring and contains only 1 heteroatom as a ring atom, the heteroatom is not a nitrogen atom.

As used herein, the terms “aryl”, “aryl ring” and “aromatic ring” are used interchangeably and refer to a fully unsaturated aliphatic hydrocarbon group. It can be an all-carbon monocyclic ring containing 6 to 14 ring atoms (i.e., C₆₋₁₄), an all-carbon polycyclic ring (the rings are connected by covalent bonds, non-fused) or an all-carbon fused polycyclic ring (i.e., ring that share adjacent pairs of carbon atoms) group, and at least one ring in the ring system is aromatic, i.e., has a conjugated π electron system. Aryl groups containing 6 to 10 ring atoms (i.e., 6- to 10-membered or C₆₋₁₀) are preferred. Each ring in the ring system contains 5 or 6 ring atoms.

In some embodiments of the present invention, “aryl” refers to a monoaryl or polyaryl ring, non-limiting examples thereof include: phenyl, biphenyl, etc.

In some embodiments of the present invention, “aryl” refers to an aromatic fused polycyclic ring, which is a polycyclic group wherein a monoaryl ring is fused to one or more monoaryl rings, non-limiting examples thereof include: naphthyl, anthracenyl, etc.

In some embodiments of the present invention, the aryl ring (e.g., monoaryl ring, preferably phenyl) described herein can be fused with one or more non-aromatic rings to form a polycyclic group, wherein the rings attached to the parent structure are aromatic rings or non-aromatic rings, and the non-aromatic rings include but are not limited to: 3- to 6-membered monocyclic heterocycloalkyl rings, preferably 5- or 6-membered monocyclic heterocycloalkyl rings (the ring carbon atoms of the monocyclic heterocycloalkyl ring can be substituted by 1 to 2 oxo groups to form a ring lactam or ring lactone structure), 3- to 6-membered monocyclic cycloalkyl rings, preferably 5- or 6-membered monocyclic cycloalkyl rings (the ring carbon atoms of the monocyclic cycloalkyl ring can be substituted by 1 or 2 oxo group(s) to form a cyclic ketone structure) etc. The polycyclic group wherein the monoaryl ring above is fused with one or more non-aromatic rings can be connected to other groups or the parent structure through nitrogen atoms or carbon atoms, the ring connected to the parent structure is a monoaryl ring or non-aromatic ring.

As used herein, the phenyl group is fused with a 5- or 6-membered monocyclic heterocycloalkyl ring to form a 9- or 10-membered bicyclic ring, which refers that a 5- or 6-membered monocyclic heterocycloalkyl ring is formed by two adjacent substituent groups on the phenyl group and the ring atoms to which they are connected, the 5- or 6-membered monocyclic heterocycloalkyl ring is as defined herein, the formed 9- or 10-membered bicyclic ring may also be called a 9- or 10-membered phenylheterocycloalkyl ring.

Herein the phenyl group is fused with a 5- or 6-membered monocyclic cycloalkyl ring to form a 9- or 10-membered bicyclic ring, which refers that a fused 5- or 6-membered monocyclic cycloalkyl ring is formed by two adjacent substituent groups on the phenyl group and the ring atoms to which they are connected, the 5- or 6-membered monocyclic cycloalkyl ring is as defined herein, the formed 9- or 10-membered bicyclic ring may also be called a 9- or 10-membered phenylcycloalkyl ring.

In the present invention, the above types of aryl groups may be substituted or unsubstituted. When substituted, the substituent is preferably one or more groups described in the present application.

As used herein, the terms “heteroaryl”, “heteroaryl ring” and “heteroaromatic ring” are used interchangeably and refer to a fully unsaturated aliphatic hydrocarbon group containing heteroatoms. It may have 5 to 14 ring atoms (i.e., 5- to 14-membered), preferably 5 to 10 ring atoms (i.e., 5- to 10-membered), more preferably 5, 6, 8, 9 or 10 ring atoms of monocyclic or fused polycyclic (i.e., rings sharing pairs of adjacent carbon atoms or heteroatoms) groups, wherein containing 1 to 4 heteroatoms as rings atoms, heteroatoms are selected from oxygen, sulfur and nitrogen. The nitrogen and sulfur atoms may be optionally oxidized, and the nitrogen atoms may be optionally quaternized. The heteroaryl group preferably has 6, 10 or 14 π electrons shared in the ring system. At least one ring of the ring system is aromatic. In some embodiments of the present invention, “heteroaryl” refers to a monocyclic heteroaryl ring (preferably a 5- or 6-membered monocyclic heteroaryl ring). Non-limiting examples of monocyclic heteroaryl include: thiophene, N-alkylpyrrolidone, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine, etc.

In some embodiments of the present invention, “heteroaryl” refers to a fused polyheteroaryl ring (preferably an 8- to 10-membered bicyclic heteroaryl ring). The fused polyheteroaryl ring includes both a polycyclic group (preferably a 9 or 10 membered bicyclic heteroaryl ring) fused by monoaryl ring (preferably a phenyl group) and a monocyclic heteroaryl ring (preferably a 5- or 6-membered monocyclic heteroaryl ring) and polycyclic group (preferably an 8- to 10-membered bicyclic heteroaryl ring) fused by a monocyclic heteroaryl group (preferably a 5- or 6-membered monocyclic heteroaryl group) and a monocyclic heteroaryl group (preferably a 5- or 6-membered monocyclic heteroaryl group).

In some embodiments of the present invention, non-limiting examples of monocyclic heteroaryl rings (preferably 5- or 6-membered monocyclic heteroaryl rings) forming fused polycyclic rings include:

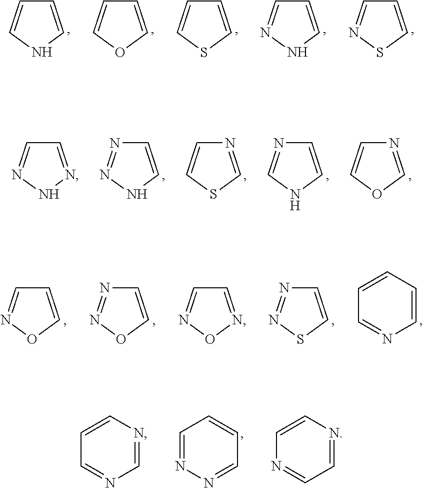

The two randomly connected ring atoms on the above monocyclic heteroaryl ring, including C—C, N-C, and N—N, can be fused with the cycloalkyl, heterocycloalkyl, aryl or heteroaryl, such as monocyclic cycloalkyl ring, monocyclic heterocycloalkyl ring, monoaryl ring, 5- or 6-membered monocyclic heteroaryl ring, etc. defined in the present invention, to form fused polycyclic rings. The two ring atoms connected to the monocyclic heteroaryl ring forming a fused ring with other rings are preferably C—C, including the following forms without limitation:

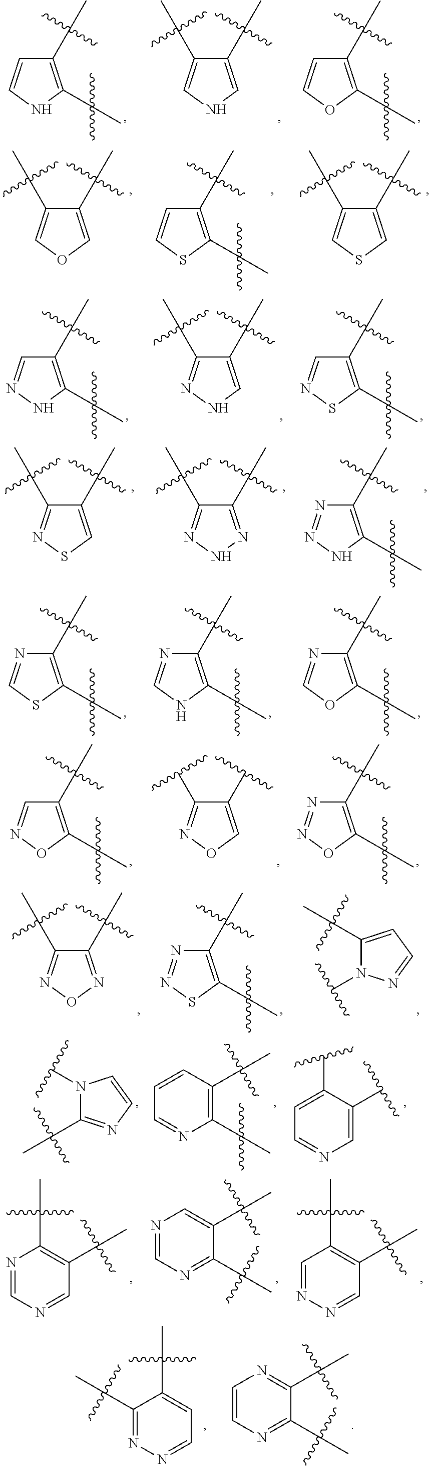

Non-limiting examples of fused polyheteroaryl rings include: benzo[d]isoxazole, 1H-indole, isoindole, 1H-benzo[d]imidazole, benzo[d]isothiazole, 1H-benzo[d][1,2,3]triazole, benzo[d]oxazole, benzo[d]thiazole, indazole, benzofuran, benzo[b]thiophene, quinoline, isoquinoline, quinazoline, quinoxaline, cinnoline, pyrido[3,2-d]pyrimidine, pyrido[2,3-d]pyrimidine, pyrido[3,4-d]pyrimidine, pyrido[4,3-d]pyrimidine, 1,8-naphthyridine, 1,7-naphthyridine, 1,6-naphthyridine, 1,5-naphthyridine, pyrazolo[1,5-a]pyrimidine , imidazo[1,2-b]pyridazine, etc.

The above monocyclic heteroaryl group, or a polycyclic group wherein a monoaryl ring is fused with a monocyclic heteroaryl ring, or a polycyclic group wherein a monocyclic heteroaryl group is fused with a monocyclic heteroaryl group can be connected to other groups or parent structures through nitrogen atoms or carbon atoms. When the group is a polycyclic group, the ring connected to the parent structure is a heteroaryl ring, an aryl ring, a monocyclic cycloalkyl ring or a monocyclic heterocycloalkyl ring, non-limiting examples thereof include:

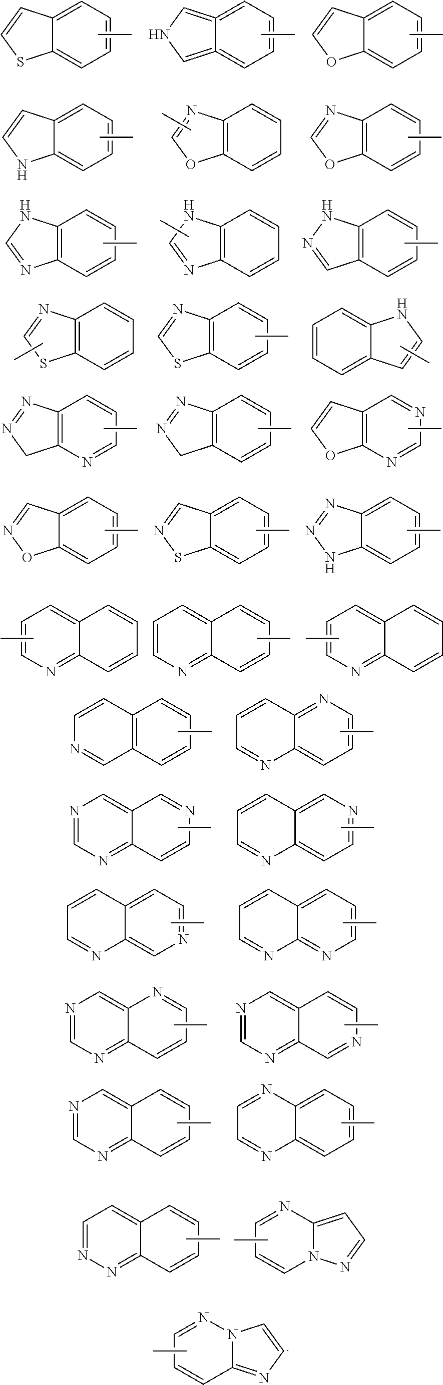

In some embodiments of the present invention, the heteroaryl ring of the present invention (e.g., a monocyclic heteroaryl ring, preferably a 5- or 6-membered monocyclic heteroaryl ring) may be fused with one or more non-aromatic rings to form polycyclic groups, wherein the ring connected to the parent structure is a heteroaryl ring or a non-aromatic ring, and the non-aromatic ring includes but is not limited to: a 3- to 6-membered (preferably 5- or 6-membered) monocyclic heterocycloalkyl ring (the ring carbon atoms of the monocyclic heterocycloalkyl ring can be substituted by 1 to 2 oxo groups to form a cyclic lactam or cyclic lactone structure), a 3- to 6-membered (preferably 5- or 6-membered) monocyclic cycloalkyl ring (the ring carbon atoms of the monocyclic cycloalkyl ring can be substituted by 1 or 2 oxo group(s) to form a cyclic ketone structure), etc.

The polycyclic group fused by above monocyclic heteroaryl ring and one or more non-aromatic rings can be connected to other groups or parent structures through nitrogen atoms or carbon atoms, the ring attached to the parent structure is a heteroaryl ring or a non-aromatic ring.

As used herein, the 5- or 6-membered monocyclic heteroaryl is fused with a 5- or 6-membered monocyclic heterocycloalkyl ring to form an 8- to 10-membered biheterocycle, which refers that a fused 5- or 6-membered monocyclic heterocycloalkyl ring is fused by the two adjacent substituent groups on the 5- or 6-membered monocyclic heteroaryl and the ring atoms to which they are connected form, and the 5- or 6-membered monocyclic heterocycloalkyl ring is as defined herein.

As used herein, the 5- or 6-membered monocyclic heteroaryl is fused with a 5- or 6-membered monocyclic cycloalkyl ring to form an 8- to 10-membered biheterocycle, which refers that a fused 5- or 6-membered monocyclic cycloalkyl ring is fused by the two adjacent substituent groups of 5- or 6-membered monocyclic heteroaryl and the ring atoms to which they are connected, and the 5- or 6-membered monocyclic cycloalkyl ring is as herein.

Non-limiting examples thereof include:

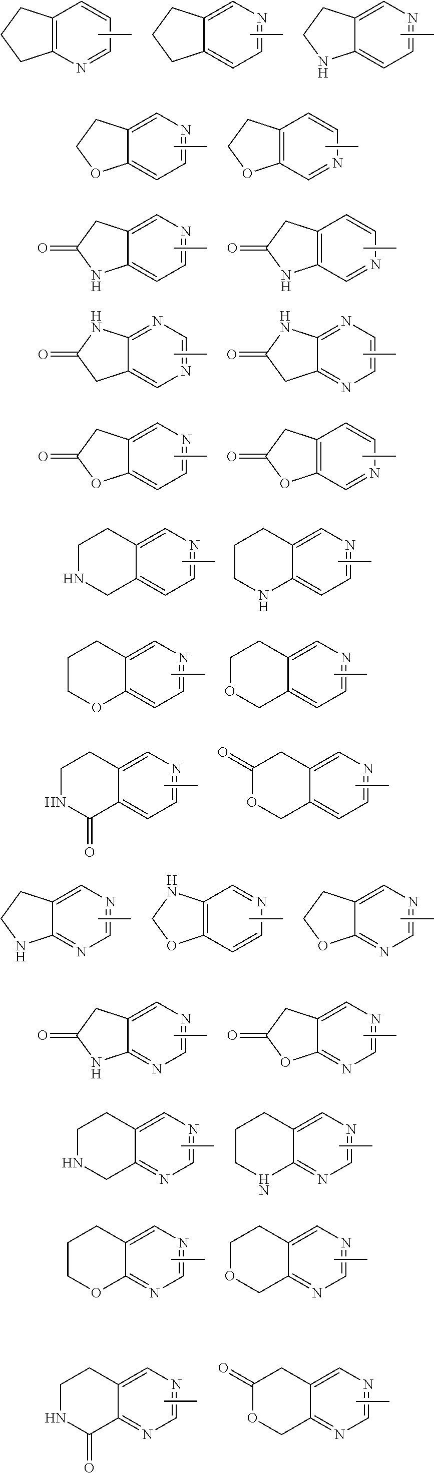

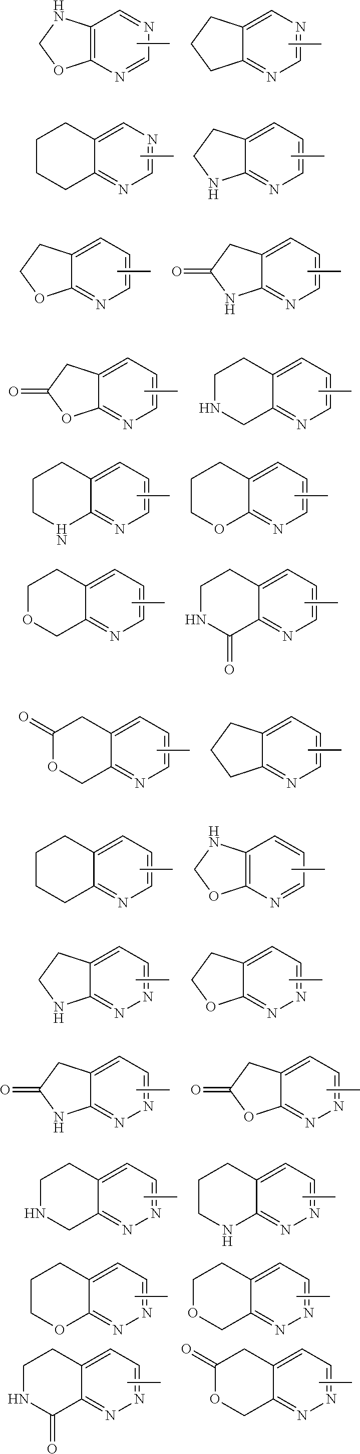

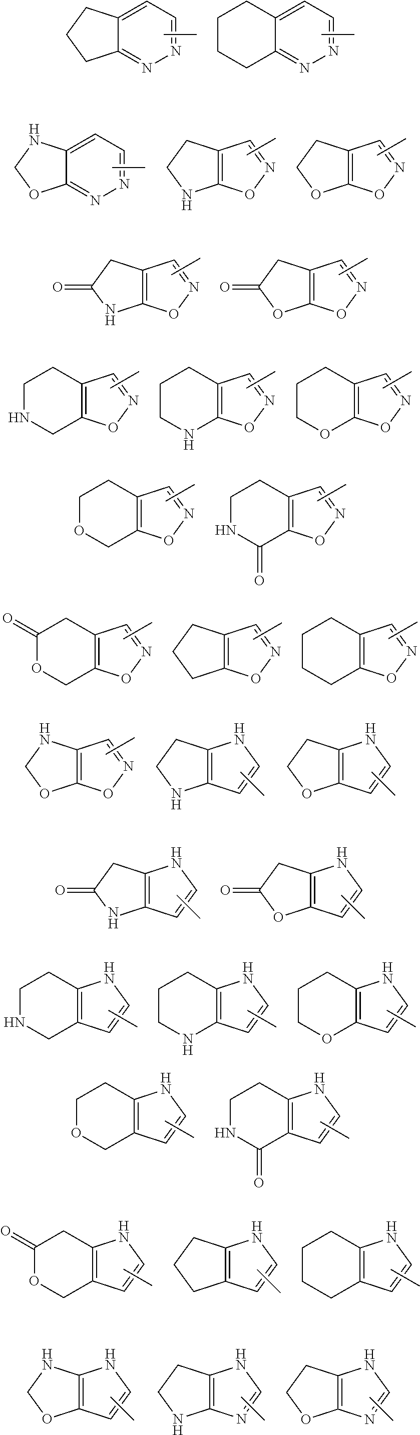

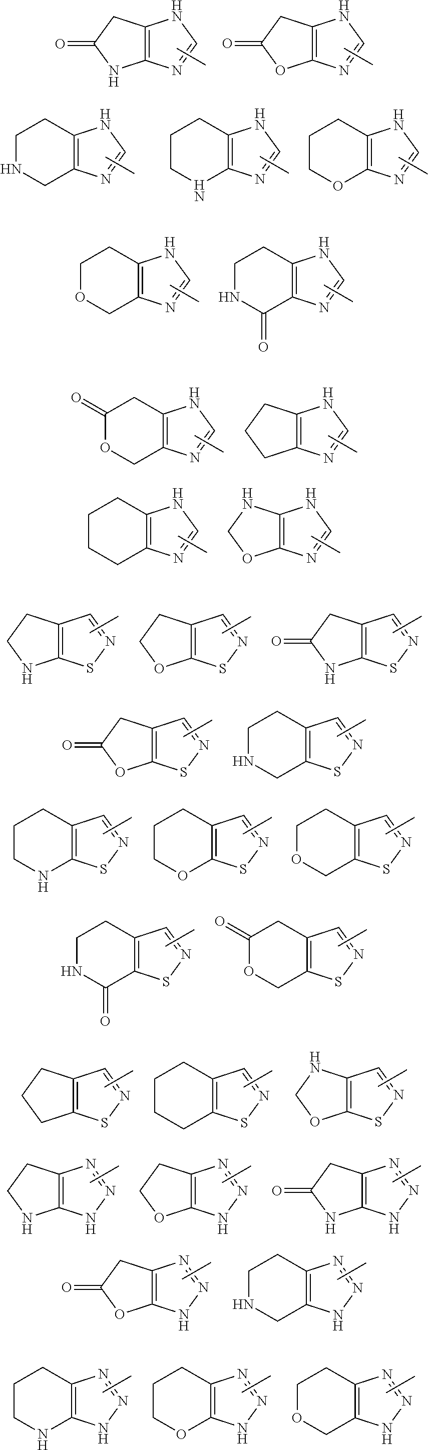

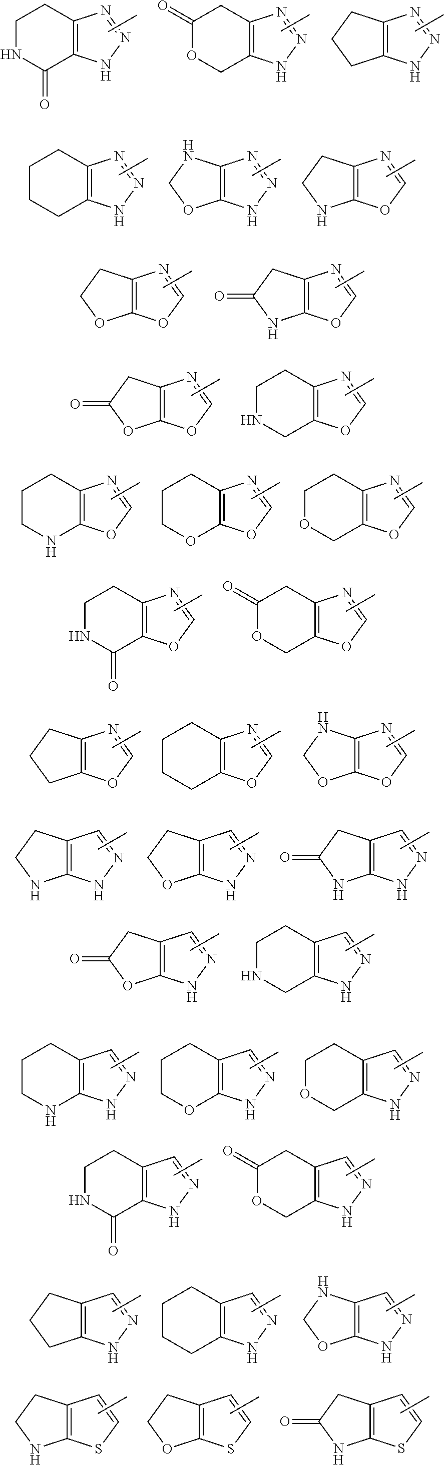

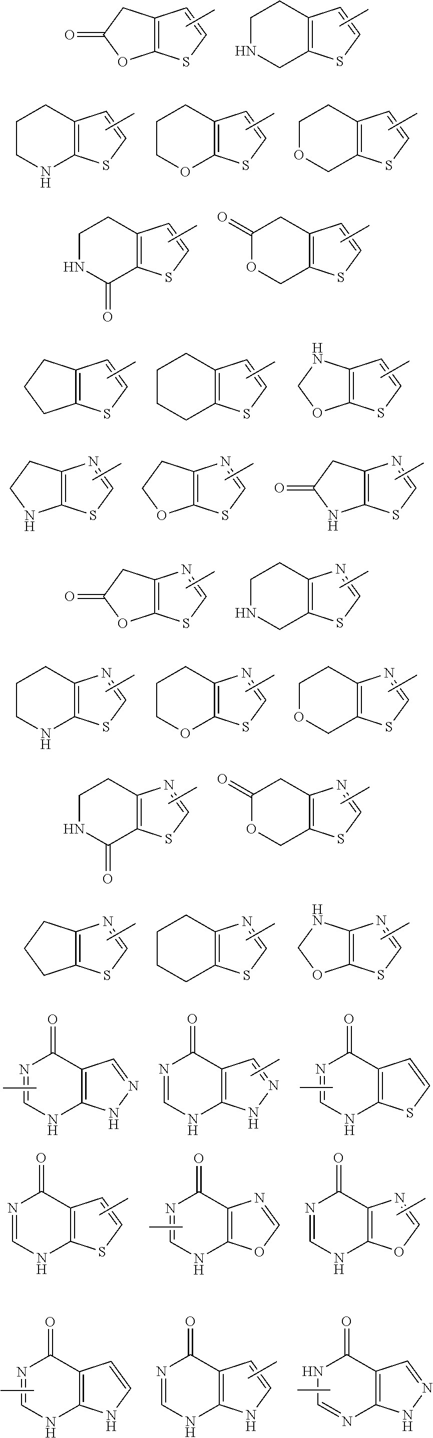

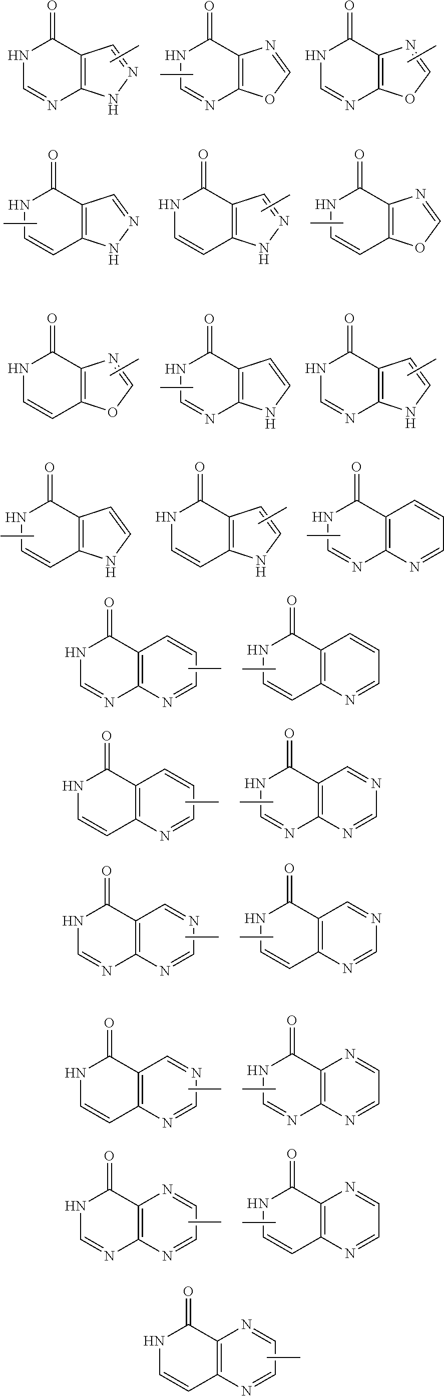

In the present invention, the above types of heteroaryl groups may be substituted or unsubstituted. When substituted, the substituent is preferably one or more groups described in the present application.

As used herein, the term “alkoxy” refers to —O-alkyl, wherein alkyl is as defined above. A C₁₋₆ alkoxy group is preferred, and a C₁₋₃ alkoxy group is more preferred. Non-limiting examples include methoxy, ethoxy, propoxy, isopropoxy, butoxy, tert-butoxy, isobutoxy, pentyloxy, etc. The alkoxy group may be optionally substituted or unsubstituted. When substituted, the substituent is preferably one or more groups described in the present application. As used herein, “deuterated” means that one or more (e.g., 1, 2, 3, 4 or 5) or all hydrogens in a group are replaced by deuterium atoms.

For example, “deuterated alkyl” refers to an alkyl group in which one or more (e.g., 1, 2, 3, 4 or 5) or all hydrogens are replaced by deuterium atoms, wherein alkyl is as defined above. A deuterated C₁₋₆ alkyl group is preferred, and a deuterated C₁₋₃ alkyl group is more preferred. For example, the deuterated methyl group may be monodeuterated methyl, dideuterated methyl or perdeuterated methyl.

As used herein, “halo” means that one or more (e.g., 1, 2, 3, 4 or 5) hydrogens in a group are replaced by halogens.

For example, “haloalkyl” refers to an alkyl group substituted by one or more (e.g., 1, 2, 3, 4 or 5) halogens, wherein alkyl is as defined above. A halogenated C₁₋₆ alkyl group is preferred, and a halogenated C₁₋₃ alkyl group is more preferred. Examples of halogenated C₁₋₈ alkyl groups include (but are not limited to) monochloromethyl, dichloromethyl, trichloromethyl, monochloroethyl, 1,2-dichloroethyl, trichloroethyl, monobromoethyl, monofluoromethyl, difluoromethyl, trifluoromethyl, monofluoroethyl, difluoroethyl, trifluoroethyl, etc.

As another example, “haloalkoxy” refers to an alkoxy group substituted by one or more (eg, 1, 2, 3, 4 or 5) halogens, wherein alkoxy is as defined above. A halogenated C₁₋₆ alkoxy group is preferred, and a halogenated C₁₋₃ alkoxy group is more preferred. It includes (but is not limited to) trifluoromethoxy, trifluoroethoxy, monofluoromethoxy, monofluoroethoxy, difluoromethoxy, difluoroethoxy, etc.

As another example, “halogenated cycloalkyl” refers to a cycloalkyl group substituted by one or more (e.g., 1, 2, 3, 4 or 5) halogens, wherein cycloalkyl is as defined above. Halogenated C₃₋₆ cycloalkyl is preferred. It includes (but is not limited to) trifluorocyclopropyl, monofluorocyclopropyl, monofluorocyclohexyl, difluorocyclopropyl, difluorocyclohexyl, etc.

As used herein, examples of “C₃₋₁₀ cycloalkenyl” include cyclopropenyl, cyclobutenyl, cyclopentenyl, cyclohexenyl, cycloheptenyl, and cyclooctenyl.

As used herein, examples of “C₆₋₁₄ aryl” include phenyl, 1-naphthyl, 2-naphthyl, 1-anthracenyl, 2-anthracenyl, and 9-anthracenyl.

As used herein, examples of “C₆₋₁₄ aryl-C₁₋₄ alkyl-” include benzyl, phenethyl, naphthylmethyl and phenylpropyl.

As used herein, examples of “optionally halogenated C₁₋₆ alkoxy” include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy and hexyloxy.

As used herein, examples of “C₁₋₆ alkylthio” include methylthio, ethylthio, propylthio, isopropylthio, butylthio, sec-butylthio, tert-butylthio, pentylthio and hexylthio.

As used herein, examples of “optionally halogenated C₁₋₆ alkylthio” include C₁₋₆ alkylthio optionally having 1 to 7 halogen atoms, preferably 1 to 5 halogen atoms. Specific examples thereof include methylthio, difluoromethylthio, trifluoromethylthio, ethylthio, propylthio, isopropylthio, butylthio, 4,4,4-trifluorobutylthio, pentylthio and hexylthio.

As used herein, examples of “C₁₋₆alkyl-C(═O)—” include acetyl, propionyl, butyryl, 2-methylpropionyl, valeryl, 3-methylbutyryl, 2-methylbutyryl, 2,2-dimethylpropionyl, hexanoyl and heptanoyl.

As used herein, examples of “optionally halogenated C₁₋₆alkyl-C(═O)—” include C-6 alkyl-C(═O)— optionally having 1 to 7 halogen atoms, preferably 1 to 5 halogen atoms. Specific examples thereof include acetyl, chloroacetyl, trifluoroacetyl, trichloroacetyl, propionyl, butyryl, valeryl and hexanoyl.

As used herein, examples of “C₁₋₆ alkoxy-C(═O)—” include methoxy-C(═O)—, ethoxy-C(═O)—, propoxy-C(═O)—, isopropoxy-C(═O)—, butoxy-C(═O)—, isobutoxy-C(═O)—, sec-butoxy-C(═O)—, tert-butoxy-C (═O)—, pentyloxy-C(═O)— and hexyloxy-C(═O)—.

As used herein, examples of “C₆₋₁₄ aryl-C(═O)—” include benzoyl, 1-naphthoyl, and 2-naphthoyl.

As used herein, examples of “C₆₋₁₄aryl-C₁₋₄-alkyl-C(═O)—” include phenylacetyl and phenylpropanoyl.

As used herein, examples of “5- to 14-membered heteroaryl-C(═O)—” include nicotinyl, isonicotinoyl, thiophenoyl, and furoyl.

As used herein, examples of “3- to 14-membered heterocycloalkyl-C(═O)—” include morpholinyl-C(═O)—, piperidinyl-C(═O)— and pyrrolidinyl-C(═O)—.

As used herein, examples of “mono- or di-C₁₋₆ alkyl-carbamoyl” include methylcarbamoyl, ethylcarbamoyl, dimethylcarbamoyl, diethylcarbamoyl, and N-ethyl-N-methylcarbamoyl.

As used herein, examples of “mono- or di-C₆₋₁₄ aryl-C₁₋₄ alkyl-carbamoyl” include benzylcarbamoyl and phenethylcarbamoyl.

As used herein, examples of “C₁₋₆ alkylsulfonyl” include methylsulfonyl, ethylsulfonyl, propylsulfonyl, isopropylsulfonyl, butanesulfonyl, sec-butylsulfonyl and tert-butylsulfonyl.

As used herein, examples of “optionally halogenated C₁₋₆ alkylsulfonyl” include C₁₋₆ alkylsulfonyl optionally having 1 to 7 halogen atoms, preferably 1 to 5 halogen atoms. Specific examples thereof include methylsulfonyl, difluoromethylsulfonyl, trifluoromethylsulfonyl, ethylsulfonyl, propylsulfonyl, isopropylsulfonyl, butanesulfonyl, 4,4,4-trifluorobutanesulfonyl, pentylsulfonyl and hexanesulfonyl.

As used herein, examples of “C₆₋₁₄ arylsulfonyl” include phenylsulfonyl, 1-naphthalenesulfonyl and 2-naphthalenesulfonyl.

As used herein, the term “hydroxy” refers to —OH.

As used herein, the term “halogen” refers to fluorine, chlorine, bromine or iodine.

As used herein, the term “amino” refers to —NH₂.

As used herein, the term “cyano” refers to —CN.

As used herein, the term “nitro” refers to —NO₂.

As used herein, the term “benzyl” refers to —CH₂-benzene.

As used herein, the term “oxo” refers to ═O.

As used herein, the term “carboxy” refers to —C(═O)OH.

As used herein, the term “formyl” refers to —C(═O)H.

As used herein (except in the embodiments), a wavy line on a group, no matter how it appears, indicates where it is connected to other parts of the molecule. If there are no wavy lines marked on the group, it means that any position in the group may be connected to other positions in the molecule.

“Optional” or “optionally” means that the subsequently described event or circumstance can but need not occur, the description includes instances where the event or circumstance does or does not occur. For example, “heterocycloalkyl group optionally substituted by alkyl” means that alkyl groups may but need not be present, and this description includes the case where the heterocycloalkyl group is substituted by an alkyl group and the heterocycloalkyl group is not substituted by an alkyl group.

“Substituted” means that one or more hydrogen atoms in a group, preferably up to 5, more preferably 1 to 3 hydrogen atoms, are independently substituted by a corresponding number of substituents. It goes without saying that the substituents are only in their possible chemical positions, and a person skilled in the art will be able to determine (either experimentally or theoretically) possible or impossible substitutions without undue effort, For example, an amino or hydroxyl group with a free hydrogen may be unstable when combined with a carbon atom with an unsaturated (e.g., olefinic) bond.

Unless otherwise defined, when a certain group described in the present invention is substituted by a substituent, it refers that all the same groups appearing in the present invention can be substituted by a substituent, which means that the group can be substituted when it exists alone, and it also means that the group can also be substituted when it exists in combination with other groups. For example, R is —C₁₋₆ alkyl, C₆₋₁₀ aryl, C₃₋₆ monocyclic cycloalkyl, —C(O)C₁₋₆ alkyl, —C₁₋₄ alkyl-C₆₋₁₀ aryl or —S(O)₂-C₃₋₆ monocyclic cycloalkyl, wherein the C₁₋₆ alkyl, C₆₋₁₀ aryl, C₃₋₆ monocyclic cycloalkyl are optionally substituted, this description also includes the C₁₋₆ alkyl, C₆₋₁₀ aryl and C₃₋₆ monocyclic cycloalkyl in —C(O)C₁₋₆ alkyl, —C₁₋₄ alkyl-C₆₋₁₀ aryl and —S(O)₂—C₃₋₆ monocyclic cycloalkyl are optionally substituted.

Unless otherwise defined, “ . . . the same or different, and each independently is . . . ” in the present invention refers that when there is more than one identical substituent group in the general formula, the groups may be the same or different and each be of independent types. For example, L is (CR_{L1}R_{L2})ₛ. When s is 2, i.e. L is (CR_{L1}R_{L2})-(CR_{L1}R_{L2}), wherein the two R_{L1} or R_{L2} can be the same or different and each be of independent types. For example, L can be C(CH₃)(CN)—C(CH₂CH₃)(OH), C(CH₃)(CN)—C(CH₃)(OH) or C(CN)(CH₂CH₃)—C(OH)(CH₂CH₃).

Unless otherwise defined, the “each independently selected from . . . (substituents)” in the present invention refer that when more than one hydrogen on the group is replaced by substituents, the types of the substituents may be the same or different, the substituents selected from are each independently of types.

As used herein, “pharmaceutical composition” means a mixture containing one or more compounds described herein, or physiologically/pharmaceutically acceptable salts or prodrugs thereof, and other chemical components, and other components for example physiologically/pharmaceutically acceptable carriers and excipients. The purpose of pharmaceutical compositions is to facilitate administration to living organisms and facilitate the absorption of active ingredients to exert biological activity.

The “pharmaceutically acceptable salts” include pharmaceutically acceptable acid addition salts and pharmaceutically acceptable base addition salts.

“Pharmaceutically acceptable acid addition salts” refer to salts formed with inorganic or organic acids that retain the biological effectiveness of the free base without other side effects.

“Pharmaceutically acceptable base addition salts” include, but are not limited to, salts of inorganic bases such as sodium salts, potassium salts, calcium salts and magnesium salts. Including but not limited to salts of organic bases, such as ammonium salts, triethylamine salts, lysine salts, arginine salts, etc.

The “solvate” mentioned in the present invention refers to the complex formed by the compound of the present invention and a solvent, They either react in the solvent or precipitate or crystallize from the solvent, For example, a complex formed with water is called a “hydrate.” The solvates of the compounds represented by formula (I) of the present invention fall within the scope of the present invention.

The compound represented by formula (I) of the present invention may contain one or more chiral centers and exist in different optically active forms. A compound contains enantiomers when it contains a chiral center. The present invention includes both isomers and mixtures of isomers, such as racemic mixtures. Enantiomers can be resolved by methods known in the art, such as crystallization and chiral chromatography. When a compound represented by formula (I) contains more than one chiral center, diastereomers may exist, The present invention includes resolved optically pure specific isomers as well as mixtures of diastereomers. Diastereomers can be resolved by methods known in the art, such as crystallization and preparative chromatography. The “stereoisomers” mentioned in the present invention include (but are not limited to) enantiomers, diastereomers, etc.

The present invention includes prodrugs of the above compounds. Prodrugs include known amino and carboxyl protecting groups and are hydrolyzed under physiological conditions or released via enzymatic reactions to yield the parent compound. Specific prodrug preparation methods can refer to (Saulnier, M. G.; Frennesson, D. B.; Deshpande, M. S.; Hansel, S. B and Vysa, D. M. Bioorg. Med. Chem Lett, 1994, 4, 1985-1990; and Greenwald, R. B.; Choe, Y. H.; Conover, C. D.; Shum, K.; Wu, D.; Royzen, M. J. Med. Chem.2000, 43, 475.).

Generally, the compounds of the present invention or pharmaceutically acceptable salts thereof, or solvates thereof, or stereoisomers thereof, or prodrugs thereof can be combined with one or more pharmaceutical carriers to form a suitable dosage form for administration. These dosage forms are suitable for oral, rectal, topical, intraoral, and other parenteral administration (e.g., subcutaneous, intramuscular, intravenous, etc.). For example, dosage forms suitable for oral administration include capsules, tablets, granules, syrups, etc. The compounds of the present invention contained in these preparations can be solid powder or granules; solutions or suspensions in aqueous or non-aqueous liquids; water-in-oil or oil-in-water emulsions, etc. The above dosage forms can be prepared from the active compound and one or more carriers or excipients through common pharmaceutical methods. The above carriers need to be compatible with the active compound or other excipients. For solid preparations, commonly used non- toxic carriers include but are not limited to mannitol, lactose, starch, magnesium stearate, cellulose, glucose, sucrose, etc. Carriers used for liquid preparations include water, physiological saline, glucose aqueous solution, ethylene glycol, polyethylene glycol, etc. The active compounds can form solutions or suspensions with the above carriers.

The compositions of the present invention are formulated, dosed, and administered in a manner consistent with good medical practice. The “therapeutically effective amount” of a compound administered is determined by factors such as the specific condition to be treated, the individual being treated, the cause of the condition, the target of the drug, and the mode of administration.

As used herein, a “therapeutically effective amount” means one that will elicit a biological or medical response in an individual, for example, an amount of a compound of the invention that reduces or inhibits enzyme or protein activity or ameliorates symptoms, alleviates a condition, slows or delays the progression of a disease, prevents a disease, etc. The therapeutically effective amount of the compound of the present invention, a pharmaceutically acceptable salt thereof, a solvate thereof, or a stereoisomer thereof contained in the pharmaceutical composition of the present invention is preferably 0.1 mg to 5 g/kg (body weight).

As used herein, “pharmaceutically acceptable carrier” means a nontoxic, inert, solid, semi-solid substance or liquid filling machine, diluent, encapsulating material or auxiliary preparation or excipient of any type that is compatible with the patient, preferably a mammal, more preferably a human. The carrier is suitable for delivering the active agent to the intended target site without terminating the activity of the agent.

As used herein, “patient” refers to an animal, preferably a mammal, and more preferably a human. The term “mammal” refers to warm-blooded vertebrate mammals, including such as cats, dogs, rabbits, bears, foxes, wolves, monkeys, deer, rats, pigs, and humans.

As used herein, “treating” means alleviating, delaying progression, attenuating, preventing, or maintaining an existing disease or condition (e.g., cancer). Treatment also includes curing, preventing the progression of, or alleviating to some degree one or more symptoms of a disease or condition.

### Embodiment

The present invention will be further described below in conjunction with specific embodiments. It should be understood that these examples are only used to illustrate the invention and are not intended to limit the scope of the invention. Experimental methods without specifying specific conditions in the following examples usually follow conventional conditions or conditions recommended by the manufacturer. Unless otherwise indicated, percentages and parts are by weight, Unless otherwise defined, terms used herein have the same meaning as is familiar to those skilled in the art, In addition, any methods and materials similar or equivalent to those described can be used in the present invention. The presence of wavy lines in the structural formulas in the following examples indicates that the compound represented by the structure is a cis-trans mixture. For example, the product in step three of Example 1.

As used herein, room temperature refers to about 20-25° C. The raw materials, reagents or solvents used in the present invention are commercially available. If no preparation method is given for the raw materials or reagents in some examples, even if the referenced preparation method is not explicitly stated, they can be obtained by referring to the methods described in other examples herein.

Abbreviation description: EA: ethyl acetate; PE: petroleum ether; MeOH: methanol; DCM: dichloromethane; i-PrOAc: isopropyl acetate; THF: tetrahydrofuran; DMF: dimethylformamide; DIEA: N,N-Diisopropylethylamine; HATU: O-(7-azabenzotriazol-1-yl)-N,N,N′,N′-tetramethyluronium hexafluorophosphate; NaHCO₃: sodium bicarbonate; NH₄Cl: ammonium chloride; HCl: hydrochloric acid; LiOH: lithium hydroxide; LiHMDS: lithium bistrimethylsilylamide; Ti(OEt)₄: tetraethyl titanate; Pd(dppf)Cl₂: [1,1′-bis(diphenylphosphino)ferrocene]palladium dichloride; TEA: triethylamine; DMAP: N,N-dimethylaniline; LDA: lithium diisopropylamide.

### EXAMPLES

### Example 1: Preparation of 2-benzyl-3-chloro-6-(2,2-difluoroethyl)-2,4,5,6-tetrahydro-7H-pyrazolo[3,4-c]pyridine-7-one(Z1)

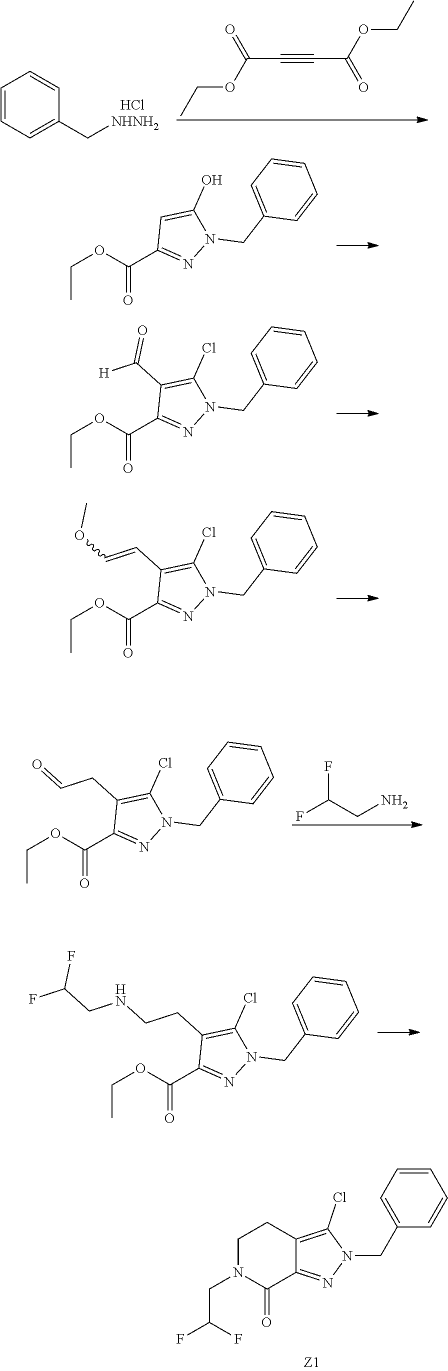 [0187] Step 1: benzylhydrazine hydrochloride (10 g, 81.853 mmol), potassium carbonate (16.969 g, 122.780 mmol), ethanol (300 mL) and diethyl butynedioate (13.928 g, 81.853 mmol) were added to a 500 mL round bottom flask, the reaction mixture was heated to 90° C. and stirred overnight, After LCMS detected that the reaction was completed, the reaction solution was cooled to 0° C., 6 M dilute hydrochloric acid (35 mL) was added, the reaction solution was extracted with ethyl acetate (300 mL×2), and the combined organic layer was washed with saturated brine (50 mL×2), dried over anhydrous sodium sulfate, the organic phase was filtered, and concentrated under reduced pressure. The residue was slurried with acetonitrile to obtain ethyl 1-benzyl-5-hydroxy-1H-pyrazole-3-carboxylate (4.67 g, yield: 30%). ES-API: [M+H]+=247.1.
Step 2: phosphorus trichloride (12.7 mL, 136 mmol) was slowly added dropwise to the solution of ethyl 1-benzyl-5-hydroxy-1H-pyrazole-3-carboxylate (4.186 g, 17.010 mmol) in N,N-dimethylformamide (5.2 mL, 68 mmol) under an ice-water bath. After the dropwise addition was completed, the reaction mixture was raised to 90° C. and stirred overnight, After LCMS detected that the reaction was completed, the reaction solution was cooled to 0° C., saturated sodium bicarbonate solution (30 mL) was added, the reaction solution was extracted with ethyl acetate (30 mL×3), and the combined organic layer was washed with saturated brine (30 mL×2), dried over anhydrous sodium sulfate, the organic phase was filtered and concentrated under reduced pressure. The crude product was purified by column chromatography (ethyl acetate: petroleum ether=0˜50%) to obtain ethyl 1-benzyl-5-chloro-4-formyl-1H-pyrazole-3-carboxylate (906 mg, Y: 18%). ES-API: [M+H]⁺=293.1.
Step 3: Potassium tert-butoxide (13.4 mL, 13.339 mmol, 1M in THF) was added to a solution of (methoxymethyl)triphenylphosphine chloride (4.679 g, 13.649 mmol) in tetrahydrofuran (30 mL). The reaction mixture was stirred for 10 minutes at 0° C. under an ice-water bath. Then a solution of ethyl 1-benzyl-5-chloro-4-formyl-1H-pyrazole-3-carboxylate (906 mg, 3.102 mmol) in tetrahydrofuran (10 mL) was added dropwise slowly to the above solution, stirred for 30 minutes at 0° C., the reaction mixture was warmed to room temperature and stirred overnight, After LCMS detected that the reaction was completed, the reaction solution was diluted with ethyl acetate (100 mL), then washed with water (20 mL×2) and saturated brine (20 mL×3). The organic phase was dried over anhydrous sodium sulfate, filtered, and concentrated under reduced pressure. The crude product was purified by column chromatography (ethyl acetate: petroleum ether=0˜50%) to obtain ethyl 1-benzyl-5-chloro-4-(2-methoxyvinyl)-1H-pyrazole-3-carboxylate (98 mg, yield: 13%), ES-API: [M+H]⁺=321.1.
Step 4: 6M dilute hydrochloric acid (1 mL, 6.00 mmol) was added to a solution of ethyl 1-benzyl-5-chloro-4-(2-methoxyvinyl)-1H-pyrazole-3-carboxylate (98 mg, 0.306 mmol) in tetrahydrofuran (5 mL) at room temperature, stirred at room temperature for 2 hours. Then 6M dilute hydrochloric acid (2 mL, 12.00 mmol) was added, and the reaction mixture was heated to 60° C. and stirred for 30 minutes. After LCMS detected that the reaction was completed, the reaction mixture was cooled to 0° C. The saturated sodium bicarbonate (30 mL) and saturated sodium chloride (30 mL) were added to the reaction solution and extracted with ethyl acetate (30 mL×3). The organic phase was dried over anhydrous sodium sulfate, filtered, and concentrated under reduced pressure. The crude product ethyl 1-benzyl-5-chloro-4-(2-oxyethyl)-1H-pyrazole-3-carboxylate (138 mg) was obtained, ES-API: [M+H]⁺=307.1.
Step 5: 2-methylpyridineborane (36.7 mg, 0.34 mmol) was added to a solution of ethyl 1-benzyl-5-chloro-4-(2-oxyethyl)-1H-pyrazole-3-ethyl carboxylate (138 mg), 2,2-difluoroethane-1-amine (36 mg), acetic acid (0.5 mL, 0.26 mmol) in methanol (5 mL, 0.26 mmol), reaction mixture was stirred at room temperature for 1 hour, under nitrogen protection. After LCMS detected that the reaction was completed, the reaction solution was quenched by adding saturated sodium bicarbonate (30 mL) and extracted with ethyl acetate (30 mL×3). The organic phase was dried with anhydrous sodium sulfate, filtered, and concentrated under reduced pressure. The crude product was purified by column chromatography (ethyl acetate: petroleum ether=0˜50%) to obtain ethyl 1-benzyl-5-chloro-4-(2-((2,2-difluoroethyl)amino)ethyl)-1H-pyrazole-3-carboxylate (114 mg, yield: 52%), ES-API: [M+H]⁺=372.1.
Step 6: trimethylaluminum toluene solution (0.76 mL, 0.758 mmol, 1M in Tol) was added to a solution of ethyl 1-benzyl-5-chloro-4-(2-((2,2-difluoroethyl)amino)ethyl)-1H-pyrazole-3-carboxylate (94 mg, 0.253 mmol) in toluene (8 mL), the reaction mixture was heated to 100° C. and stirred for 4 hours, under nitrogen protection. After LCMS detected that the reaction was completed. The reaction mixture was cooled to room temperature. Water (30 mL), ethyl acetate (30 mL), and saturated potassium sodium tartrate solution (30 mL) were added to the reaction solution in sequence. The organic phase was separated, dried over anhydrous sodium sulfate, and filtered, concentrate under reduced pressure. The crude product was purified by column chromatography (ethyl acetate: petroleum ether=0˜50%) to obtain light yellow solid 2-benzyl-3-chloro-6-(2,2-difluoroethyl)-2,4,5,6-tetrahydro-7H-pyrazolo[3,4-c]pyridin-7-one (Z1, 45 mg, yield: 48%). ES-API: [M+H]⁺=326.1. ¹H NMR (400 MHZ, CDCl₃) δ 7.30-7.26 (m, 5 H), 6.12-5.82 (m, 1 H), 5.36 (s, 2 H), 3.85-3.77 (m, 2 H), 3.68-3.65 (m, 2 H), 2.75-2.72 (m, 2 H).

### Example 2: Preparation of 3-chloro-6-(2,2-difluoroethyl)-2-(2-fluorobenzyl)-2,4,5,6-tetrahydro-7H-pyrazolo[3,4-c]pyridin-7-one(Z2)

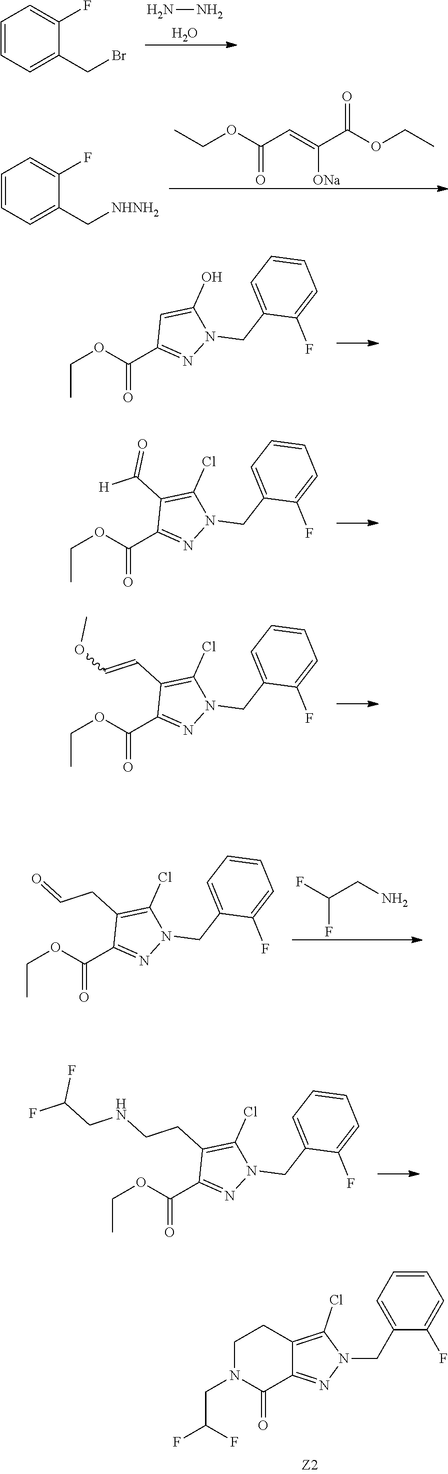 [0194] Step 1: 1-(bromomethyl)-2-fluorobenzene (14.1 g, 0.075 mol) was dissolved in ethanol (70 mL), hydrazine hydrate (18.6 g, 0.37 mol) was added, and the reaction solution was heated to 60° C. and the reaction was proceeded for 1 hour. After the reaction was completed, the reaction solution was spin-dried and purified by column chromatography (dichloromethane/methanol=0˜10/1) to obtain the product (2-fluorobenzyl)hydrazine (3 g, yield: 28%). ES-API: [M+H]+=141.0.
Step 2: diethyl oxaloacetate sodium salt (1.6 g, 7.5 mmol) was dissolved in acetic acid (10 mL), the reaction solution was reacted at room temperature for 0.5 h, and (2-fluorobenzyl)hydrazine (2.1 g, 0.15 mol) was added, the reaction solution was heated to 100° C. and the reaction was proceeded for 2 hours. After the reaction was completed, the reaction solution was extracted with ethyl acetate (100 mL) and hydrochloric acid (2N, 50 mL), dried over anhydrous sodium sulfate, evaporated to dryness, and purified by column chromatography (petroleum ether/ethyl acetate=10/1˜1/1) to obtain the product ethyl 1-(2-fluorobenzyl)-5-hydroxy-1H-pyrazole-3-carboxylate (1.2 g, yield: 61%). ES-API: [M+H]⁺=265.0. ¹H NMR (400 MHZ, cd₃od) δ 7.30 (dd, J=13.4, 6.1 Hz, 1H), 7.13-7.06 (m, 2H), 7.01 (t, J=7.2 Hz, 1H), 5.92 (s, 1H), 5.27 (s, 2H), 4.30 (q, J=7.1 Hz, 2H), 1.34 (t, J=7.1 Hz, 3H).
Step 3: ethyl 1-(2-fluorobenzyl)-5-hydroxy-1H-pyrazole-3-carboxylate (2.5 g, 9.47 mmol) was dissolved in N,N-dimethylformamide (6 mL), phosphorus oxychloride (20 mL) was added dropwise, the reaction solution was heated to 90° C. and the reaction was proceeded for 3 hours. After the reaction was completed, the reaction solution was spin-dried, quenched with water (200 mL), adjusted to pH 7˜8 with NaHCO₃ (solid), extracted with ethyl acetate (100 mL×3), dried over anhydrous sodium sulfate, spin-dried, and purified by column chromatography (petroleum ether/ethyl acetate=10/1˜5/1) to obtain the product ethyl 5-chloro-1-(2-fluorobenzyl)-4-formyl-1H-pyrazole-3-carboxylate (1.7 g, yield: 57.1%). ES-API: [M+H]⁺=311. ¹H NMR(400 MHz, CDCl₃) δ 10.43 (s, 1H), 7.36-7.28 (m, 1H), 7.14-7.05 (m, 3H), 5.53 (s, 2H), 4.47 (q, J=7.1 Hz, 2H), 1.43 (t, J=7.1 Hz, 3H).
Step 4: ethyl 5-chloro-1-(2-fluorobenzyl)-4-formyl-1H-pyrazole-3-carboxylate (1.32 g, 4.26 mmol) and (methoxymethyl)triphenylphosphine chloride (8.76 g, 25.55 mmol) was dissolved in tetrahydrofuran (100 mL), potassium tert-butoxide (2.15 g, 19.17 mmol) was added under ice bath, and then the reaction was proceeded at room temperature for 1 hour. The reaction solution was quenched with NH₄Cl solution and extracted with ethyl acetate (50 mL×3). The organic phase was dried over anhydrous sodium sulfate, spin-dried, and purified by column chromatography (petroleum ether/ethyl acetate=0˜1/3) to obtain the product ethyl 5-chloro-1-(2-fluorobenzyl)-4-(2-methoxyvinyl)-1H-pyrazole-3-carboxylate (0.6 g, yield: 41.2%). ES-API: [M+H]⁺=339.0.
Step 5: ethyl 5-chloro-1-(2-fluorobenzyl)-4-(2-methoxyvinyl)-1H-pyrazole-3-carboxylate (0.6 g, 1.78 mmol) was dissolved in tetrahydrofuran (10 mL), hydrochloric acid (6 N, 10 mL) was added, the reaction solution was heated to 50° C. and the reaction was proceeded for 3 hours. After the reaction was completed, the reaction solution was extracted with ethyl acetate/water, dried over anhydrous sodium sulfate, spin-dried, and purified by column chromatography (petroleum ether/ethyl acetate=10/1˜1/1) to obtain the product ethyl 5-chloro-1-(2-fluorobenzyl)-4-(2-oxyethyl)-1H-pyrazole-3-carboxylate (160 mg, yield: 27%). ES-API: [M+H]⁺=325.0.
Step 6: ethyl 5-chloro-1-(2-fluorobenzyl)-4-(2-oxyethyl)-1H-pyrazole-3-carboxylate (200 mg, 0.62 mmol) was dissolved in methanol (3 mL), 2,2-difluoroethylamine (60 mg, 0.74mmol) and borane-2-methylpyridine complex (100 mg, 0.93mmol) was added, and the reaction was proceeded at room temperature for 1 hour. After the reaction was completed, the reaction solution was extracted with ethyl acetate/water, dried over anhydrous sodium sulfate, spin-dried, and purified by column chromatography (petroleum ether/ethyl acetate=5/1˜1/1) to obtain the product ethyl 5-chloro-4-(2-((2,2-difluoroethyl)amino)ethyl)-1-(2-fluorobenzyl)-1H-pyrazole-3-carboxylate (0.2 g, yield: 82%). ES-API: [M+H]⁺=390.0.
Step 7: ethyl 5-chloro-4-(2-((2,2-difluoroethyl)amino)ethyl)-1-(2-fluorobenzyl)-1H-pyrazole-3-carboxylate (200 mg, 0.51 mmol) was dissolved in tetrahydrofuran (3 mL) and methanol (1 mL), lithium hydroxide monohydrate (65 mg, 1.54 mmol) and water (1 mL) were added, and the reaction was proceeded at room temperature overnight, The reaction solution was spin-dried and purified by thin layer chromatography (petroleum ether/ethyl acetate=2/1) to obtain 3-chloro-6-(2,2-difluoroethyl)-2-(2-fluorobenzyl)-2,4,5,6-tetrahydro-7H-pyrazolo[3,4-c] pyridin-7-one (Z2, 10 mg, yield: 5%). ES-API: [M+H]⁺=344.0. ¹H NMR (400 MHz, CDCl₃) δ 7.29 (dd, J=10.5, 4.7 Hz, 1H), 7.16 -7.02 (m, 3H), 6.01 (tt, J=56.2, 4.5 Hz, 1H), 5.47 (s, 2H), 3.85 (td, J=14.0, 4.5 Hz, 2H), 3.72 (t, J=6.6 Hz, 2H), 2.79 (t, J=6.6 Hz, 2H).

### Example 3: Preparation of 3-chloro-2-(2,6-difluorobenzyl)-6-(2,2-difluoroethyl)-2,4,5,6-tetrahydro-7H-pyrazole [3,4-c]pyridin-7-one(Z3)

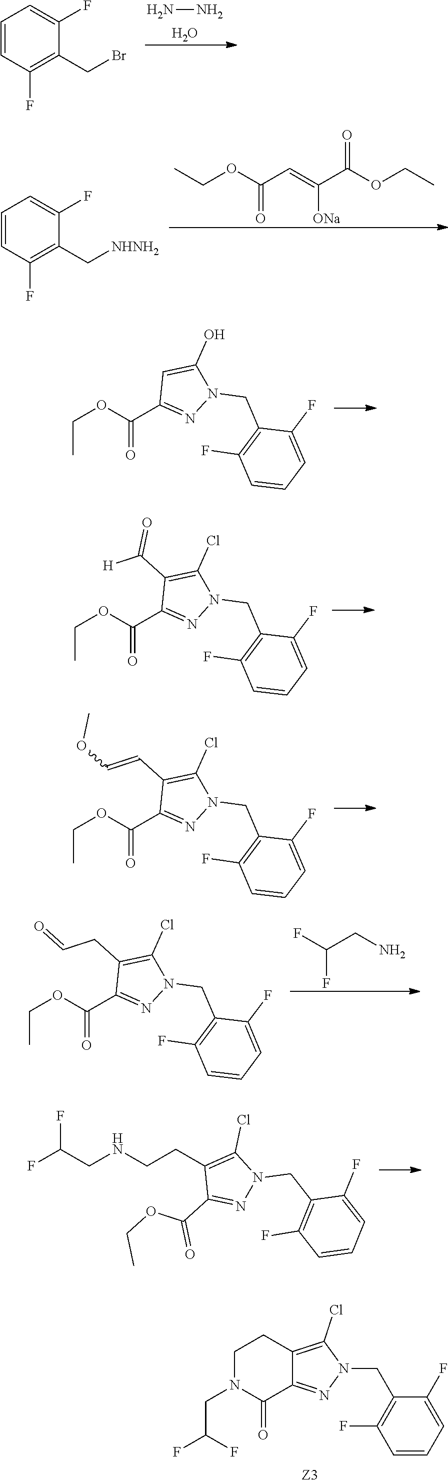 [0202] Step 1: 2-(bromomethyl)-1,3-difluorobenzene (6.2 g, 0.03 mol) was dissolved in ethanol (100 mL), hydrazine hydrate (9.3g, 0.15 mol) was added, and the reaction solution was heated to 60° C., the reaction was proceeded for 1 hour. After the reaction was completed, the product was spin-dried and purified by column chromatography (dichloromethane/methanol=0˜10/1) to obtain the product (2,6-difluorobenzyl)hydrazine (3.5 g, yield: 73%). ES-API: [M+H]⁺=159.0.
Step 2: diethyl oxaloacetate sodium salt (2.3 g, 0.011 mol) was dissolved in acetic acid (25 mL), the reaction solution was reacted at room temperature for 0.5 h, (2,6-difluorobenzyl) hydrazine (3.5 g, 0.022 mol) was added, the reaction solution was heated to 100° C. for 2 hours. After the reaction was completed, the reaction solution was extracted with ethyl acetate (100 mL) and hydrochloric acid (2N, 50 mL) to obtain the product ethyl 1-(2,6-difluorobenzyl)-5-hydroxy-1H-pyrazole-3-carboxylate (1.3 g, yield: 42%). ES-API: [M+H]⁺=283.0.
Step 3: ethyl 1-(2,6-difluorobenzyl)-5-hydroxy-1H-pyrazole-3-carboxylate (1.3g, 4.61mmol) was dissolved in N,N-dimethylformamide (3 mL), phosphorus oxychloride (10 mL) was added dropwise, the reaction solution was heated to 90° C. and the reaction was proceeded for 3 hours. After the reaction was completed, the reaction solution was spin-dried, quenched with water (200 mL), adjusted to pH 7˜8 with NaHCO₃ (solid), extracted with ethyl acetate (100 mL×3), dried over anhydrous sodium sulfate, spin-dried, and purified by column chromatography (petroleum ether/ethyl acetate=10/1˜5/1) to obtain the product ethyl 5-chloro-1-(2,6-difluorobenzyl)-4-formyl-1H-pyrazole-3-carboxylate (240 mg, yield: 22%). ES-API: [M+H]⁺=329.
Step 4: ethyl 5-chloro-1-(2,6-difluorobenzyl)-4-formyl-1H-pyrazole-3-carboxylate (240 mg, 0.73mmol) and (methoxymethyl)triphenylphosphine chloride (1.5 g, 4.39 mmol) were dissolved in tetrahydrofuran (10 mL), and potassium tert-butoxide (370 mg, 3.29 mmol) was added under ice bath, and then the reaction was proceeded at room temperature for 1 hour. The reaction solution was quenched with NH₄Cl solution and extracted with ethyl acetate (50 mL×3). The organic phase was dried over anhydrous sodium sulfate, spin-dried, and purified by column chromatography (petroleum ether/ethyl acetate=0˜1/3) to obtain the product ethyl 5-chloro-1-(2,6-difluorobenzyl)-4-(2-methoxyvinyl)-1H-pyrazole-3-carboxylate (0.23 g, yield: 88%). ES-API: [M+H]⁺=357.0.
Step 5: ethyl 5-chloro-1-(2,6-difluorobenzyl)-4-(2-methoxyvinyl)-1H-pyrazole-3-carboxylate (0.23g, 0.65 mmol) was dissolved in tetrahydrofuran (3 mL), hydrochloric acid (6 N, 3 mL) was added, the reaction solution was heated to 50° C. and the reaction was proceeded for 3 hours. After the reaction was completed, the reaction solution was extracted with ethyl acetate/water, dried over anhydrous sodium sulfate, spin-dried, and purified by column chromatography (petroleum ether/ethyl acetate=10/1˜1/1) to obtain the product ethyl 5-chloro-1-(2,6-difluorobenzyl)-4-(2-oxyethyl)-1H-pyrazole-3-carboxylate (220 mg, yield: 98%). ES-API: [M+H]⁺=343.0.
Step 6: ethyl 5-chloro-1-(2,6-difluorobenzyl)-4-(2-oxyethyl)-1H-pyrazole-3-carboxylate (220 mg, 0.64 mmol) was dissolved in methanol (3 mL), 2,2-difluoroethylamine (63 mg, 0.77 mmol) and borane-2-methylpyridine complex (103 mg, 0.96 mmol) was added, the reaction was proceeded at room temperature for 1 hour. After the reaction was completed, the reaction solution was extracted with ethyl acetate and water, dried over anhydrous sodium sulfate, spin-dried, and purified by column chromatography (petroleum ether/ethyl acetate=5/1˜1/1) to obtain the product ethyl 5-chloro-1-(2,6-difluorobenzyl)-4-(2-((2,2-difluoroethyl)amino)ethyl)-1H-pyrazole-3-carboxylate (0.2 g, yield: 82%). ES-API: [M+H]⁺=408.0.
Step 7: ethyl 5-chloro-1-(2,6-difluorobenzyl)-4-(2-((2,2-difluoroethyl)amino)ethyl)-1H-pyrazole-3-carboxylate (200 mg, 0.51 mmol) was dissolved in tetrahydrofuran (3 mL) and methanol (1 mL), lithium hydroxide monohydrate (65 mg, 1.54 mmol) and water (1 mL) were added, and the reaction was proceeded at room temperature overnight, The reaction solution was spin-dried and purified by thin layer chromatography (petroleum ether/ethyl acetate=2/1) to obtain 3-chloro-2-(2,6-difluorobenzyl)-6-(2,2-difluoroethyl)-2,4,5,6-tetrahydro-7H-pyrazolo[3,4-c]pyridin-7-one (Z3, 13 mg, yield: 6%). ES-API: [M+H]⁺=362.0. ¹H NMR (400 MHZ, CDCl₃) δ 7.31 (dd, J=14.8, 8.4 Hz, 1H), 6.90 (t, J=7.9 Hz, 2H), 6.14-5.82 (m, 1H), 5.46 (s, 2H), 3.83 (td, J=14.0, 4.5 Hz, 2H), 3.70 (t, J=6.6 Hz, 2H), 2.78 (t, J=6.6 Hz, 2H).

### Example 4: Preparation of 3-(3-chloro-2-(2,6-difluorobenzyl)-7-oxo-2,4,5,7-tetrahydro-6H-pyrazolo[3,4-c]pyridine-6-yl)propionitrile (Z4)

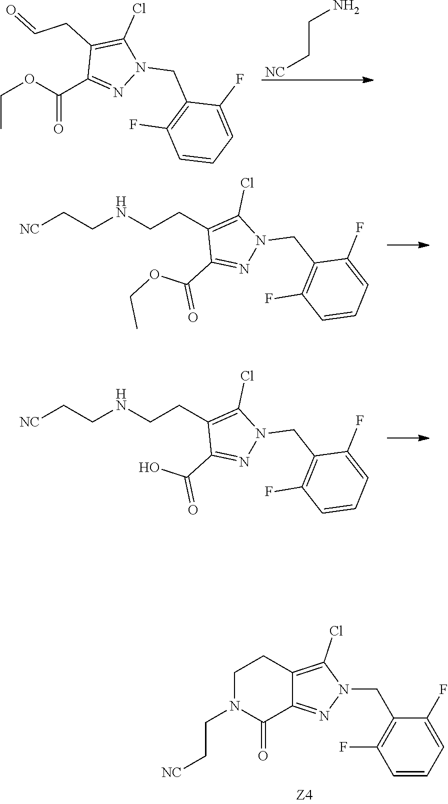 [0210] Step 1: ethyl 5-chloro-1-(2,6-difluorobenzyl)-4-(2-oxoethyl)-1H-pyrazole-3-carboxylate (250 g, 0.731 mmol) was dissolved in methanol (3 mL), 3-aminopropionitrile (102 mg, 1.46 mmol) and 2-methylpyridine borane complex (156 mg, 1.46 mmol) were added, and the reaction was proceeded at 25° C. for 5 hours. The reaction solution was quenched by adding water, extracted with dichloromethane, the organic phase was washed with saturated brine, dried over anhydrous sodium sulfate, concentrated under reduced pressure, and purified by column chromatography (petroleum ether/ethyl acetate=1/1) to obtain ethyl 5-chloro-4-(2-((2-cyanoethyl)amino)ethyl)-1-(2,6-difluorobenzyl)-1H-pyrazole-3-carboxylate (80 mg, yield: 27.6%). ES-API: [M+H]⁺=397.1.
Step 2: ethyl 5-chloro-4-(2-((2-cyanoethyl)amino)ethyl)-1-(2,6-difluorobenzyl)-1H-pyrazole-3-carboxylate (80 mg, 0.202 mmol) was dissolved in tetrahydrofuran/water (1 mL/1 mL), lithium hydroxide monohydrate (17 mg, 0.404 mmol) was added, and the reaction solution was heated to 40° C. and the reaction was proceeded for 1 hour. The reaction solution was cooled to room temperature, adjusted to pH about 5 with 1N HCl, extracted with ethyl acetate. The organic phase was washed with brine, dried over anhydrous sodium sulfate, and concentrated under reduced pressure to obtain 5-chloro-4-(2-((2-cyanoethyl)amino)ethyl)-1-(2,6-difluorobenzyl)-1H-pyrazole-3-carboxylic acid (80 mg, crude). ES-API: [M+H]⁺=369.1.
Step 3: 5-chloro-4-(2-((2-cyanoethyl)amino)ethyl)-1-(2,6-difluorobenzyl)-1H-pyrazole-3-carboxylic acid (80 mg, 0.202 mmol) was dissolved in dichloromethane (5 mL), heated to 40° C. and the reaction was proceeded for 0.5 hours. The solvent was spin-dried and the crude product was purified by thin layer chromatography (petroleum ether/ethyl acetate=1/1) to obtain 3-(3-chloro-2-(2,6-difluorobenzyl)-7-oxo-2,4 ,5,7-tetrahydro-6H-pyrazolo[3,4-c]pyridin-6-yl)propionitrile (Z4, 4.6 mg, yield: 6.5%). ¹H NMR (400 MHZ, CDCl₃) 87.34-7.27 (m, 1H), 6.90 (t, J=7.9 Hz, 2H), 5.46 (s, 2H), 3.77 (td, J=6.5, 3.6 Hz, 4H), 2.83 (t, J=6.6 Hz, 2H), 2.72 (t, J=6.2 Hz, 2H). ES-API: [M+H]⁺=351.1.

### Example 5: Preparation of 3-chloro-2-(2,5-difluorobenzyl)-6-(2,2-difluoroethyl)-2,4,5,6-tetrahydro-7H-pyrazolo[3,4-c]pyridin-7-one(Z5)

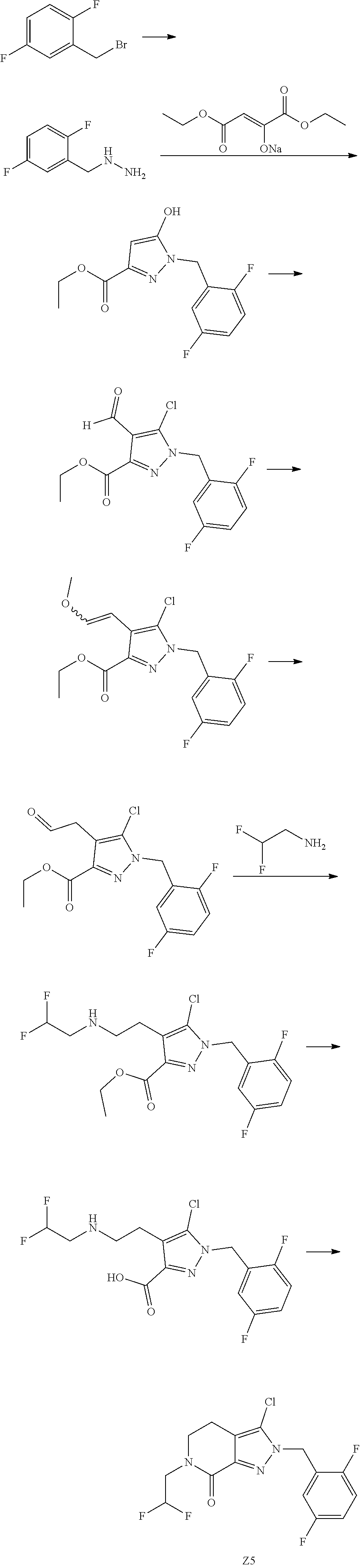 [0214] Step 1: hydrazine hydrate (12.1 g, 0.24 mol) was dissolved in ethanol (100 mL), the temperature was raised to 50° C., a solution of 2,5-difluorobenzyl bromide (10 g, 0.48 mol) in ethanol (100 mL) was added dropwise. After the addition was completed, the reaction was proceeded for 0.5 hours with heat preserved. The reaction solution was directly spin-dried, purified by column chromatography (dichloromethane:methanol=10:1) to obtain colorless oily substance (2,5-difluorobenzyl)hydrazine (7 g, yield: 72%), which was directly used for the next reaction. ES-API: [M+1]⁺=159.0.
Step 2: diethyl oxaloacetate sodium salt (4.6 g, 0.022 mol) was dissolved in acetic acid (200 mL), (2,5-difluorobenzyl)hydrazine (7 g, 0.044 mol) was added, heated to 100° C. and the reaction was proceeded for 2 hours. The reaction solution was spin-dried and purified by column chromatography (petroleum ether: ethyl acetate=1:1) to obtain ethyl 1-(2,5-difluorobenzyl)-5-hydroxy-1H-pyrazole-3-carboxylate (7 g, yield: 100%). ES-API: [M+1]⁺=283.0.
Step 3: ethyl 1-(2,5-difluorobenzyl)-5-hydroxy-1H-pyrazole-3-carboxylate (7 g, 0.025 mol) was added with N,N-dimethylformamide (7 mL), phosphorus oxychloride (100 mL) respectively, the temperature was raised to 100° C. and the reaction was proceeded for 18 hours. The reaction solution was poured into water (300 mL) after most of the phosphorus oxychloride was spin-dried, adjusted with sodium bicarbonate solid to weak alkalinity and extracted with ethyl acetate (100 mL×3), dried over anhydrous sodium sulfate, spin-dried and purified by column chromatography (petroleum ether: ethyl acetate=5:1) to obtain ethyl 5-chloro-1-(2,5-difluorobenzyl)-4-formyl-1H-pyrazole-3-carboxylate (3.2 g, yield: 40%) ES-API: [M+1]⁺=329.0. ¹H NMR (400 MHz, CDCl₃) δ 10.44 (s, 1H), 7.11-6.97 (m, 2H), 6.77 (ddd, J=8.5, 5.7, 3.1 Hz, 1H), 5.50 (s, 2H), 4.49 (d, J=7.1 Hz, 2H), 1.44 (t, J=7.1 Hz, 3H).
Step 4: (methoxymethyl)triphenylphosphine chloride (4.92 g, 14.35 mmol) and potassium tert-butoxide (1.47 g, 13.08 mmol) were added to the reaction bottle, tetrahydrofuran (100 mL) was added. The reaction solution was cooled to 0° C., and ethyl 5-chloro-1-(2,5-difluorobenzyl)-4-formyl-1H-pyrazole-3-carboxylate (1.02 g, 3.12 mmol) was added, the reaction was proceeded for 1 hour with heat preserved. The reaction solution was poured into water (200 mL), extracted with ethyl acetate (100 mL×3), dried over anhydrous sodium sulfate, spun to dryness, and purified by column chromatography (petroleum ether: ethyl acetate=3:1) to obtain ethyl 5-chloro-1-(2,5-difluorobenzyl)-4-(2-methoxyvinyl)-1H-pyrazole-3-carboxylate (510 mg, yield: 45.8%). ES-API: [M+1]⁺=357.0.
Step 5: ethyl 5-chloro-1-(2,5-difluorobenzyl)-4-(2-methoxyvinyl)-1H-pyrazole-3-carboxylate (300 mg, 0.84 mmol) was dissolved in tetrahydrofuran (2 mL), HCl (5N, 2 mL) was added, the temperature was raised to 50° C., and the reaction was proceeded for 1 hour. The reaction solution was poured into water (50 mL), extracted with ethyl acetate (20 mL×3), spin-dried to obtain ethyl 5-chloro-1-(2,5-difluorobenzyl)-4-(2-oxoethyl)-1H-pyrazole-3-carboxylate (300 mg, yield: 100%), which was directly used in the next reaction.
Step 6: ethyl 5-chloro-1-(2,5-difluorobenzyl)-4-(2-oxoethyl)-1H-pyrazole-3-carboxylate (300 mg, 0.87 mmol), 2,2-difluoroethylamine (78 mg, 0.96 mmol), 2-methylpyridine-N-borane (140 mg, 1.31 mmol) were added to methanol (5 mL) at once and stirred at room temperature for 1 hour. The reaction solution was poured into water (100 mL), extracted with ethyl acetate (20 mL×3), spin-dried, and purified by column chromatography (petroleum ether: ethyl acetate=4:1) to obtain ethyl 5-chloro-1-(2,5-difluorobenzyl)-4-(2-((2,2-difluoroethyl)amino)ethyl)-1H-pyrazole-3-carboxylate (86 mg, yield: 23%). ES-API: [M+23]⁺=408.0.
Step 7: lithium hydroxide monohydrate (25 mg, 0.61mol) was dissolved in water (0.2 mL) and added dropwise to a solution of ethyl 5-chloro-1-(2,5-difluorobenzyl)-4-(2-((2,2-difluoroethyl)amino)ethyl)-1H-pyrazole-3-carboxylate (86 mg, 0.2 mmol) in tetrahydrofuran (1 mL) and the reaction was proceeded at room temperature for 2 hours after the addition was completed. The reaction solution was adjusted pH˜6 with HCl (2N), extracted with ethyl acetate (20 mL×2), spin-dried and purified by thin layer chromatography (petroleum ether/ethyl acetate=2/1) to obtain 3-chloro-2-(2,5-difluorobenzyl)-6-(2,2-difluoroethyl)-2,4,5,6-tetrahydro-7H-pyrazolo[3,4-c]pyridin-7-one (Z5, 10 mg, yield: 13.8%). ES-API: [M+23]⁺=362.0. ¹H NMR (400 MHz, CDCl₃) δ 7.04 (td, J=9.0, 4.4 Hz, 1H), 6.97 (ddd, J=9.0. 7.4, 3.6 Hz, 1H), 6.77 (ddd, J=8.6, 5.7, 3.1 Hz, 1H), 6.02 (tt, J=56.2, 4.5 Hz, 1H), 5.45 (s, 2H), 3.87 (td, J=14.0, 4.5 Hz, 2H), 3.74 (t, J=6.6 Hz, 2H), 2.81 (t, J=6.6 Hz, 2H).

### Example 6: Preparation of 3-chloro-6-(2,2-difluoroethyl)-2-(2,3,6-trifluorobenzyl)-2,4,5,6-tetrahydro-7H-Pyrazolo[3,4-c]pyridin-7-one(Z6)

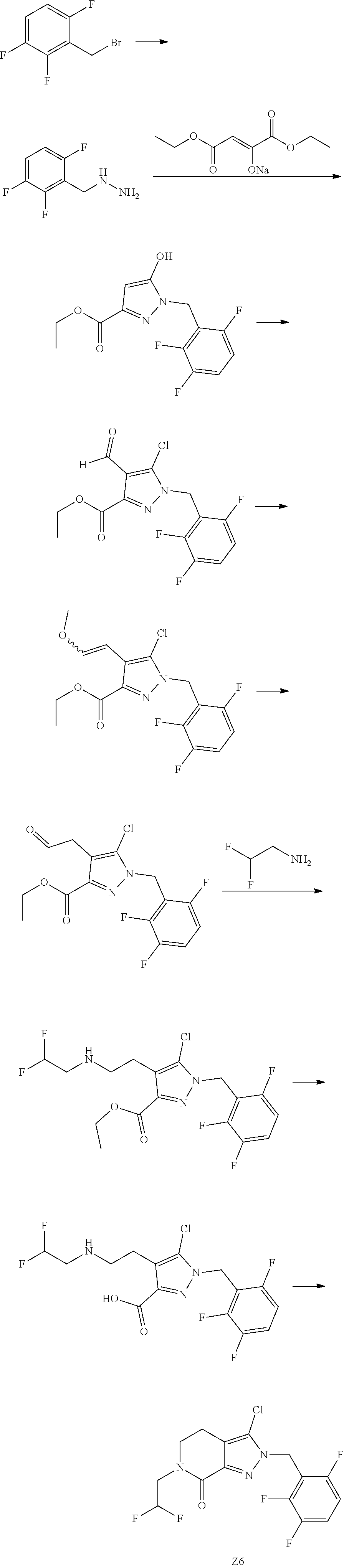 [0222] Step 1: hydrazine hydrate (5.5 g, 0.11 mol) was dissolved in ethanol (100 mL), the temperature was raised to 50° C., a solution of 1,3,4 trifluorobenzyl bromide (5 g, 0.022 mol) in ethanol (100 mL) was added dropwise, and the reaction was proceeded for 0.5 hours with heat preserved after the addition was completed. The reaction solution was spin-dried and purified by column chromatography (dichloromethane:methanol=10:1) to obtain a colorless oily substance (2,3,6-trifluorobenzyl)hydrazine (3.16 g, yield: 81%)), which was used directly for the next reaction. ES-API: [M+1]⁺=177.0.
Step 2: diethyl oxaloacetate sodium salt (1.9 g, 8.98 mmol) was dissolved in acetic acid (50 mL), (2,3,6-trifluorobenzyl)hydrazine (3.16 g, 17.95 mmol) was added, and the reaction was proceeded at 100° C. for 2 hours. The reaction solution was spin-dried and purified by column chromatography (petroleum ether: ethyl acetate=1:1) to obtain ethyl 5-hydroxy-1-(2,3,6-trifluorobenzyl)-1H-pyrazole-3-carboxylate (3.4 g, yield: 63%). ES-API: [M+1]⁺=301.0.
Step 3: ethyl 5-hydroxy-1-(2,3,6-trifluorobenzyl)-1H-pyrazole-3-carboxylate (3.4 g, 11.3 mmol) was added to N,N-dimethylformamide (4 mL), phosphorus oxychloride (50 mL) respectively, the reaction solution was heated to 100° C. and the reaction was proceeded for 18 hours. The reaction solution was poured into water (200 mL) after most of the phosphorus oxychloride was spin-dried, adjusted with sodium bicarbonate solid to weak alkalinity, extracted with ethyl acetate (100 mL×3), dried with anhydrous sodium sulfate, spin-dried and purified by column chromatography (petroleum ether: ethyl acetate=5:1) to obtain ethyl 5-chloro-4-formyl-1-(2,3,6-trifluorobenzyl)-1H-pyrazole-3-carboxylate (2 g, yield: 53.7%). ES-API: [M+1]⁺=347.0. ¹H NMR (400 MHz, CDCl₃) δ 10.40 (s, 1H), 7.19 (qd, J=9.2, 5.1 Hz, 1H), 6.93-6.84 (m, 1H), 5.53 (s, 2H), 4.45 (q, J=7.1 Hz, 2H), 1.41 (t, J=7.1 Hz, 3H).
Step 4: (methoxymethyl)triphenylphosphonium chloride (2 g, 5.71 mmol) and potassium tert-butoxide (586 mg, 5.21 mmol) was added into the reaction flask, tetrahydrofuran (50 mL) was added, and cooled to 0° C. Ethyl 5-chloro-4-formyl-1-(2,3,6-trifluorobenzyl)-1H-pyrazole-3-carboxylate (430 mg, 1.24 mmol) was added, and the reaction was proceeded for 1 hour with heat preserved after the addition was completed. The reaction solution was poured into water (200 mL), extracted with ethyl acetate (100 mL×3), dried over anhydrous sodium sulfate, spin-dried, and purified by column chromatography (petroleum ether: ethyl acetate=3:1) to obtain ethyl 5-chloro-4-(2-methoxyvinyl)-1-(2,3,6-trifluorobenzyl)-1H-pyrazole-3-carboxylate (220 mg, yield: 46%). ES-API: [M+1]⁺=375.0.
Step 5: ethyl 5-chloro-4-(2-methoxyvinyl)-1-(2,3,6-trifluorobenzyl)-1H-pyrazole-3-carboxylate (220 mg, 0.59 mmol) was dissolved in tetrahydrofuran (2 mL), HCl (5N, 2 mL) was added, and the reaction was proceeded for 1 hour after the temperature was raised to 50° C. The reaction solution was poured into water (50 mL), extracted with ethyl acetate (20 mL×3), spin-dried to obtain ethyl 5-chloro-4-(2-oxoethyl)-1-(2,3,6-trifluorobenzyl)-1H-pyrazole-3-carboxylate (210 mg, yield: 98%), which was directly used in the next reaction. ES-API: [M+1]⁺=361.0.
Step 6: ethyl 5-chloro-4-(2-oxoethyl)-1-(2,3,6-trifluorobenzyl)-1H-pyrazole-3-carboxylate (210 mg, 0.58 mmol), 2,2-difluoroethylamine (52 mg, 0.64 mmol), 2-methylpyridine-N-borane (81 mg, 0.75 mmol) were added to methanol (3 mL) in sequence and stirred at room temperature for 1 hour. The reaction solution was poured into water (50 mL), extracted with ethyl acetate (20 mL×3), dried with anhydrous sodium sulfate, spin-dried and purified by column chromatography (petroleum ether: ethyl acetate=4:1) to obtain ethyl 5-chloro-4-(2-((2,2-difluoroethyl)amino)ethyl)-1-(2,3,6-trifluorobenzyl)-1H-pyrazole-3-carboxylate (200 mg, yield: 81%). ES-API: [M+1]⁺=426.0.
Step 7: lithium hydroxide monohydrate (59 mg, 1.41 mol) was dissolved in water (1 mL) and added dropwise to a solution of ethyl 5-chloro-4-(2-((2,2-difluoroethyl)amino)ethyl)-1-(2,3,6-trifluorobenzyl)-1H-pyrazole-3-carboxylate (200 mg, 0.47 mmol) in tetrahydrofuran (3 mL), the reaction was proceeded at room temperature for 2 hour after the addition was completed. The reaction solution was adjusted with HCl (2N) to PH˜6, extracted with ethyl acetate (20 mL×2), spin-dried, and purified by thin layer chromatography (petroleum ether/ethyl acetate=2/1) to obtain 5-chloro-4-(2-((2,2-difluoroethyl)amino)ethyl)-1-(2,3,6-trifluorobenzyl)-1H-pyrazole-3-carboxylic acid (45 mg, yield: 24%). ES-API: [M+1]⁺=398.0.
Step 8: 5-chloro-4-(2-((2,2-difluoroethyl)amino)ethyl)-1-(2,3,6-trifluorobenzyl)-1H-pyrazole-3-carboxylic acid (45 mg, 0.11 mmol), O-(7-azabenzotriazol-1-yl)-N,N,N′,N′-tetramethyluronium hexafluorophosphate (52 mg, 0.14 mmol), N,N-diisopropylethylamine (29 mg, 0.22 mmol) were added to N,N-dimethylformamide (2 mL) in sequence, and the reaction was proceeded at room temperature for 0.5 hours. The reaction solution was poured into water (50 mL), extracted with ethyl acetate (20 mL×3) and purified by thin layer chromatography (petroleum ether/ethyl acetate=2/1) to obtain white solid 3-chloro-6-(2,2-difluoroethyl)-2-(2,3,6-trifluorobenzyl)-2,4,5,6-tetrahydro-7H-pyrazolo[3,4-c]pyridin-7-one (Z6, 8 mg, yield: 19%). ES-API: [M+1]⁺⁼380.0. ¹H NMR (400 MHz, CDCl₃) δ 7.14 (qd, J=9.2, 5.0 Hz, 1H), 6.90-6.80 (m, 1H), 5.98 (tt, J=56.3, 4.5 Hz, 1H), 5.46 (s, 2H), 3.82 (td, J=14.0, 4.5 Hz, 2H), 3.70 (t, J=6.6 Hz, 2H), 2.79 (t, J=6.6 Hz, 2H).

### Example 7: Preparation of 2-(1-(3-chloro-2-(2,6-difluorobenzyl)-7-oxo-2,4,5,7-tetrahydro-6H-pyrazolo[3,4-c]pyridin-6-yl)cyclopropyl)acetonitrile (Z7)

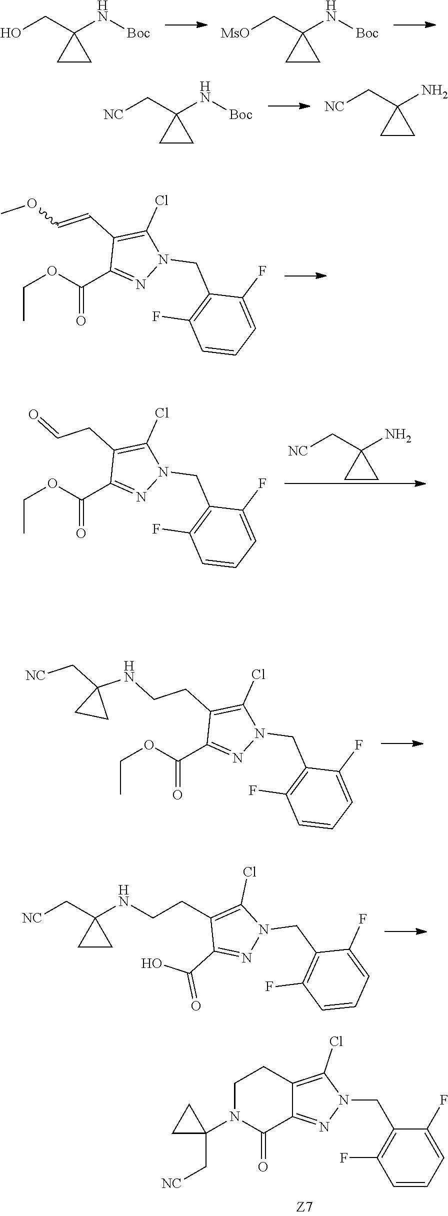 [0231] Step 1: 1-(hydroxymethyl)cyclopropyl)carbamic acid tert-butyl ester (1000 mg, 5.35 mmol) was dissolved in dichloromethane (10 mL), triethylamine (1.08 g, 10.7 mmol) was added, methylsulfonyl chloride (0.918 g, 8.02 mmol) was added under ice water bath, and the reaction was proceeded at room temperature for 2 hours. The reaction solution was washed with 1N dilute hydrochloric acid and saturated brine in sequence, dried over anhydrous sodium sulfate, and concentrated under reduced pressure to obtain (1-((tert-butoxycarbonyl)amino)cyclopropyl)methylmethylsulfonate (1.3 g, yield: 91.7%), which was directly used in the next reaction. ES-API: [M+H]⁺⁼266.1.
Step 2: (1-((tert-butoxycarbonyl)amino)cyclopropyl)methylmethylsulfonate (300 mg, 1.132 mmol) and sodium cyanide (111 mg, 2.26 mmol) were dissolved in N,N-dimethyl sulfoxide, heated to 50° C. and the reaction was proceeded for 3 hours. The reaction solution was cooled to room temperature, with water added, extracted with ethyl acetate. The organic phase was washed with saturated brine, dried over anhydrous sodium sulfate, concentrated under reduced pressure, and purified by column chromatography (petroleum ether/ethyl acetate=3/1) to obtain (1-(cyanomethyl)cyclopropyl)carbamic acid tert-butyl ester (180 mg, yield: 81%). ES-API: [M+H]⁺⁼197.1.
Step 3: (1-(cyanomethyl)cyclopropyl)carbamic acid tert-butyl ester (180 mg, 0.918 mmol) was dissolved in dichloromethane (2 mL) and trifluoroacetic acid (2 mL), and the reaction was proceeded at room temperature for 1 hour. The solution was concentrated under reduced pressure until the solvent was dried, adjusted to pH=12 with 1N sodium hydroxide solution, extracted with dichloromethane. The organic phase was washed with saturated brine, dried over anhydrous sodium sulfate, and concentrated under reduced pressure to obtain 2-(1-aminocyclopropyl)acetonitrile (50 mg, yield: 56.8%). ES-API: [M+H]⁺⁼97.1.
Step 4: ethyl 5-chloro-1-(2,6-difluorobenzyl)-4-(2-methoxyvinyl)-1H-pyrazole-3-carboxylate (600 mg, 1.69 mmol) was dissolved in 5N hydrochloric acid (6 mL) and tetrahydrofuran (6 mL), the reaction was proceeded at 50° C. for 1 hour. The reaction solution was cooled to room temperature, with water added, extracted with ethyl acetate. The organic phase was washed with saturated brine, dried over anhydrous sodium sulfate, and concentrated under reduced pressure to obtain ethyl 5-chloro-1-(2,6-difluorobenzyl)-4-(2-oxoethyl)-1H-pyrazole-3-carboxylate (600 mg, crude product). ES-API: [M+H]⁺⁼343.1.
Step 5: ethyl 5-chloro-1-(2,6-difluorobenzyl)-4-(2-oxoethyl)-1H-pyrazole-3-carboxylate (178 mg, 0.52 mmol) was dissolved in methanol (2 mL), 2-(1-aminocyclopropyl)acetonitrile (50 mg, 0.52 mmol) and 2-methylpyridine borane complex (8 mg, 0.78 mmol) were added, and the reaction was proceeded at 25° C. for 5 hours. The reaction solution was quenched by adding water, extracted with dichloromethane. The organic phase was washed with saturated brine, dried over anhydrous sodium sulfate, concentrated under reduced pressure, and purified by column chromatography (petroleum ether/ethyl acetate=3/1) to obtain ethyl 5-chloro-4-(2-((1-(cyanomethyl)cyclopropyl)amino)ethyl)-1-(2,6-difluorobenzyl)-1H-pyrazole-3-carboxylate (105 mg, yield: 47.9%). ES-API: [M+H]⁺⁼423.1.
Step 6: ethyl 5-chloro-4-(2-((1-(cyanomethyl)cyclopropyl)amino)ethyl)-1-(2,6-difluorobenzyl)-1H-pyrazole-3-carboxylate (105 mg, 0.249 mmol) was dissolved in tetrahydrofuran/water (1 mL/1 mL), lithium hydroxide monohydrate (2 mg, 0.498 mmol) were added, heated to 40° C. and the reaction was proceeded for 1 hour. The reaction solution was cooled to room temperature, adjusted to pH around 5 with 1N HCl, and concentrated under reduced pressure to obtain 5-chloro-4-(2-((1-(cyanomethyl)cyclopropyl)amino)ethyl)-1-(2,6-difluorobenzyl)-1H-pyrazole-3-carboxylic acid (200 mg, crude). ES-API: [M+H]⁺⁼395.1.
Step 7: 5-chloro-4-(2-((1-(cyanomethyl)cyclopropyl)amino)ethyl)-1-(2,6-difluorobenzyl)-1H-pyrazole-3-carboxylic acid (200 mg, 0.508 mmol), N,N-diisopropylethylamine (164 mg, 1.27 mmol), O-(7-azabenzotriazol-1-yl)-N,N,N′,N′-tetramethyluronium hexafluorophosphate (231 mg, 0.609 mmol) was dissolved in N,N-dimethylformamide (2 mL) and the reaction was proceeded at 20° C. for 1 hour. The reaction solution was washed with 0.5N dilute hydrochloric acid, washed with saturated brine, dried over anhydrous sodium sulfate, spin-dried to dry the solvent, and purified by thin layer chromatography (petroleum ether/ethyl acetate=1/1) to obtain 2-(1-(3-chloro-2-(2,6-difluorobenzyl)-7-oxo-2,4,5,7-tetrahydro-6H-pyrazolo[3,4-c]pyridin-6-yl)cyclopropyl)acetonitrile (Z7, 20 mg, yield: 10.5%).¹H NMR (400 MHz, CDCl₃) 87.34-7.26 (m, 1H), 6.95-6.83 (m, 2H), 5.43 (s, 2H), 3.78 (t, J=6.5 Hz, 2H), 2.73 (t, J=6.5 Hz, 4H), 1.04 (s, 4H). ES-API: [M+H]⁺⁼377.1.

### Example 8: Preparation of 3-chloro-2-(2-chlorobenzyl)-6-(2,2-difluoroethyl)-2,4,5,6-tetrahydro-7H-pyrazolo[3,4-c]pyridin-7-one (Z8)

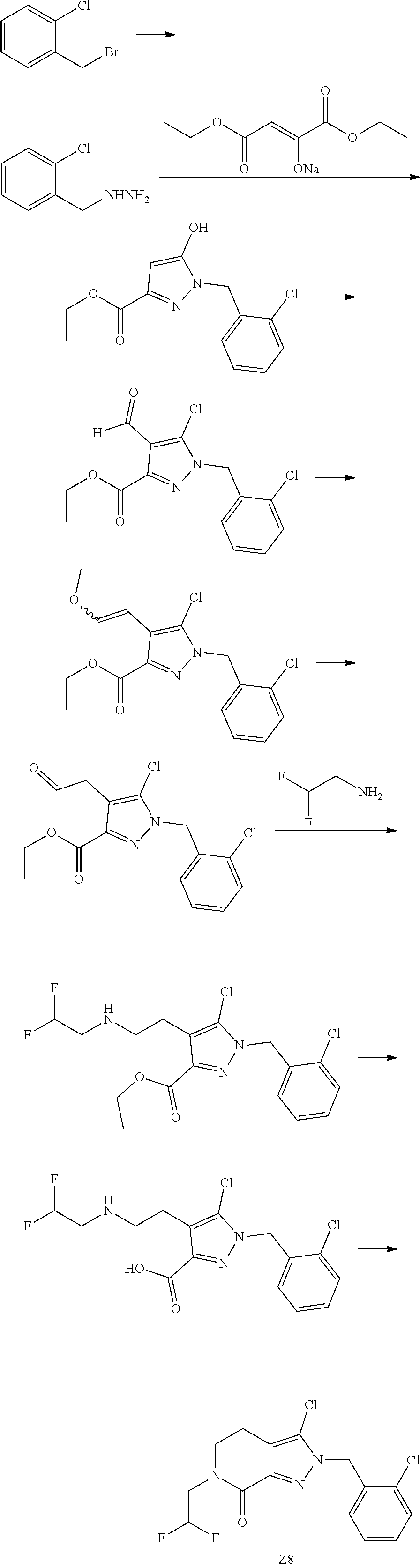 [0239] Step 1: hydrazine hydrate (12 g, 243 mmol) was heated to 60° C., a solution of 2-chlorobromobenzene (5.0 g, 24.3 mmol) in ethanol (120 mL) was added dropwise, and continued stirring for 1 hour after the addition. The reaction solution was cooled to room temperature, and the excess hydrazine hydrate and ethanol was removed by rotary evaporation. The crude product was purified by column chromatography (dichloromethane/methanol=40/1-10/1) to obtain 2-chlorobenzylhydrazine (3.8g, yield: 98%). ES-API: [M+H]⁺⁼157.1.
Step 2: diethyl oxaloacetate sodium salt (3.83 g, 18.2 mmol) was dissolved in acetic acid (60 mL), and 2-chlorobenzylhydrazine (3.8 g, 24.3 mmol) was added. The reaction was proceeded at 100° C. for 16 hours. The reaction solution was rotary evaporated to remove the solvent, the residue was dissolved in dichloromethane, washed with sodium bicarbonate solution, and the solvent was evaporated. The crude product was purified by column chromatography (petroleum ether/ethyl acetate=7/1-1/1) to obtain ethyl 1-(2-chlorobenzyl)-5-hydroxy-1H-pyrazole-3-carboxylate (3.6 g, yield: 71%). ES-API: [M+H]⁺⁼281.1.
Step 3: ethyl 1-(2-chlorobenzyl)-5-hydroxy-1H-pyrazole-3-carboxylate (4.0 g, 14.3 mmol) was dissolved in phosphorus oxychloride (30 mL), N,N-dimethylformamide (4 mL) was added. The reaction was proceeded at 90° C. for 16 hours. The phosphorus oxychloride was removed by rotary evaporation, the residue was diluted with dichloromethane, washed with sodium bicarbonate solution, extracted with dichloromethane, and the solvent was spun off. The crude product was purified by column chromatography (petroleum ether/ethyl acetate=10/1-4/1) to obtain ethyl 5-chloro-1-(2-chlorophenyl)-4-formyl-1H-pyrazole-3-carboxylate (3.24 g, yield: 64%). ES-API: [M+H]+=327.0.
Step 4: Under nitrogen protection, methoxymethyltriphenylphosphine chloride (4.8 g, 14.06 mmol) and potassium tert-butoxide (1.44 g, 12.8 mmol) was suspended in anhydrous tetrahydrofuran (25 mL). The reaction solution was cooled to 0° C., and the solution of ethyl 5-chloro-1-(2-chlorophenyl)-4-formyl-1H-pyrazole-3-carboxylate (1 g, 3.05 mmol) in tetrahydrofuran (25 mL) was added dropwise. Under stirring, the reaction was proceeded for 1 hour, slowly raised to room temperature, quenched with saturated ammonium chloride solution, extracted with ethyl acetate, and the solvent was removed by rotary evaporation. The crude product was purified by column chromatography (petroleum ether/ethyl acetate=10/1-4/1) to obtain ethyl 5-chloro-1-(2-chlorobenzyl)-4-(2-methoxyvinyl)-1H-pyrazole-3-carboxylate (350 mg, yield: 32%). ES-API: [M+H]⁺⁼355.1.
Step 5: ethyl 5-chloro-1-(2-chlorobenzyl)-4-(2-methoxyvinyl)-1H-pyrazole-3-carboxylate (35 mg, 1.0 mmol) was dissolved in tetrahydrofuran (4 mL), hydrochloric acid (5N, 3 mL) was added. The reaction was proceeded at 40° C. for 1 hour. The reaction solution was extracted with ethyl acetate, dried over anhydrous sodium sulfate, and the solvent was evaporated to obtain ethyl 5-chloro-1-(2-chlorobenzyl)-4-(2-oxoethyl)-1H-pyrazole-3-carboxylate (250 mg, yield: 74%).
Step 6: ethyl 5-chloro-1-(2-chlorobenzyl)-4-(2-oxoethyl)-1H-pyrazole-3-carboxylate (250 mg, 0.74 mmol) was dissolved in methanol (5 mL), wherein 2-methylpyridine borane complex (102 mg, 0.96 mmol) and 2,2-difluoroethylamine (59 mg, 0.74 mmol) were added. Under the reaction solution was stirring, the reaction was proceeded at room temperature for 1 hour. The reaction solution was extracted with ethyl acetate, dried over anhydrous sodium sulfate, and the solvent was rotary evaporated. The crude product was purified by column chromatography (petroleum ether/ethyl acetate=8/1-1/1) to obtain ethyl 5-chloro-1-(2-chlorobenzyl)-4-(2-((2,2-difluoroethyl)amino)ethyl)-1H-pyrazole-3-carboxylate (220 mg, yield: 74%). ES-API: [M+H]⁺⁼406.1.
Step 7: ethyl 5-chloro-1-(2-chlorobenzyl)-4-(2-((2,2-difluoroethyl)amino)ethyl)-1H-pyrazole-3-carboxylate (20 mg, 5.43 mmol) was dissolved in tetrahydrofuran/methanol/water (4 mL/1 mL/1 mL). The reaction was proceeded at room temperature for 1 hour. The reaction solution was acidified with dilute hydrochloric acid (2N, pH=6-7). The solvent was removed by rotary evaporation to obtain 5-chloro-1-(2-chlorobenzyl)-4-(2-((2,2-difluoroethyl)amino)ethyl)-1H-pyrazole-3-carboxylic acid (220 mg, crude product). ES-API: [M+H]⁺⁼378.0.
Step 8: 5-chloro-1-(2-chlorobenzyl)-4-(2-((2,2-difluoroethyl)amino)ethyl)-1H-pyrazole-3-carboxylic acid (220 mg, crude product) was dissolved in dichloromethane (3 mL), wherein HATU (265 mg, 0.70 mmol) and N,N-diisopropylethylamine (224 mg, 1.74 mmol) were added. The reaction was proceeded at room temperature for 1 hour. The reaction solution was extracted with dichloromethane, dried over anhydrous sodium sulfate, and the solvent was rotary evaporated. The crude product was purified by column chromatography (petroleum ether/ethyl acetate=6/1-1/1) to obtain 3-chloro-2-(2-chlorobenzyl)-6-(2,2-difluoroethyl)-2,4,5,6-tetrahydro-7H-pyrazolo[3,4-c]pyridin-7-one (Z8, 60 mg, yield: 29%). ¹H NMR (400 MHz, CDCl₃) δ 7.39 (dd, J=7.9, 1.1 Hz, 1H), 7.25-7.15 (m, 2H), 6.84 (d, J=7.7 Hz, 1H), 6.03 (tt, J=56.2, 4.5 Hz, 1H), 5.55 (s, 2H), 3.87 (td, J=14.0, 4.5 Hz, 2H), 3.75 (t, J=6.6 Hz, 2H), 2.83 (t, J=6.6 Hz, 2H). ES-API: [M+H]⁺⁼360.0.

### Example 9: Preparation of 3-chloro-2-(2,6-difluorobenzyl)-6-(1,1-difluoropropan-2-yl)-2,4,5,6-tetrahydro-7H-pyrazolo[3,4-c]pyridin-7-one (Z9) and Isomers Thereof

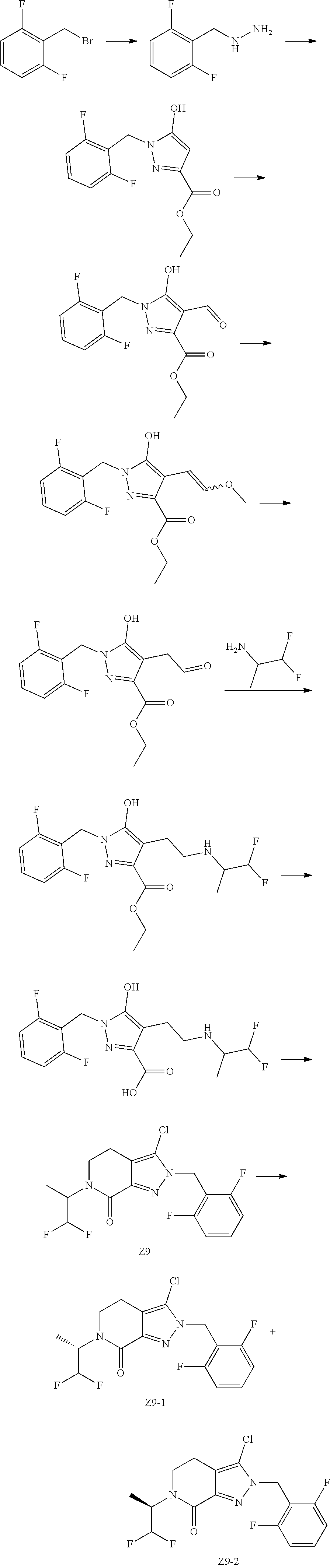 [0248] Step 1: hydrazine hydrate (36.1 g, 721 mmol, 5 eq.) was placed in a 500 mL three-neck flask and stirred at 50° C. for 30 minutes. 2-(bromomethyl)-1,3-difluorobenzene (30 g, 144 mmol, 1 eq) was dissolved in 300 mL of anhydrous ethanol and slowly added to the flask dropwise. After the addition was completed, the reaction was proceeded at 50-60° C. for 1 hour. After TLC (PE) showed that the raw material reacted completely, the reaction solvent was spin-dried under reduced pressure, and the crude product was purified by column chromatography (MeOH/DCM=1/20) to obtain 21 g of colorless oil (2,6-difluorobenzyl)hydrazine, yield: 92%. ES-API: [M+H]⁺=159.07.
Step 2: sodium (Z)-1,4-diethoxy-1,4-dioxobut-2-en-2-ol (13.9 g, 66 mmol) was dissolved in acetic acid (200 mL) and stirred for 30 minutes, the reaction solution became clear. (2,6-difluorobenzyl)hydrazine (21 g, 132 mmol) was added to the reaction solution, heated to 100° C., and the reaction was proceeded for 16 hours. The reaction solvent was spin-dried under reduced pressure, dissolved in ethyl acetate (400 mL), washed with saturated sodium bicarbonate solution (50 mL×2), and washed twice with water (50 mL×2). The organic phases were combined, sequentially, dried over anhydrous sodium sulfate, concentrated, and the crude product was purified by column chromatography (MeOH/DCM=1/20) to obtain a yellow solid ethyl 1-(2,6-difluorobenzyl)-5-hydroxy-1H-pyrazole-3-carboxylate (14 g, yield: 75%). ES-API: [M+H]⁺=282.08.
Step 3: ethyl 1-(2,6-difluorobenzyl)-5-hydroxy-1H-pyrazole-3-carboxylate (14 g, 49.6 mmol) was dissolved in DMF (14 mL), phosphorus oxychloride (10 mL) was added dropwise, heated to 90° C. after addition was completed and the reaction was proceeded overnight, After the reaction solution was evaporated to dryness under reduced pressure, it was slowly added into water, and the temperature was controlled within 30° C. (by adding ice). The reaction solution was adjusted to pH=7-8 with solid sodium bicarbonate, extracted with ethyl acetate (200 mL×3). The organic phase was washed with water (50 ml×1) and saturated NaCl (50 mL×1), dried over anhydrous sodium sulfate, and evaporated to dryness. The crude product was purified by column chromatography (EA/PE=0˜50%) to obtain a yellow solid ethyl 5-chloro-1-(2,6-difluorobenzyl)-4-formyl-1H-pyrazole-3-carboxylate (10 g, yield: 61.5%). ES-API: [M+H]⁺=328.04.
Step 4: (methoxymethyl)triphenylphosphine chloride (15.34 g, 44.76 mmol, 4.6 eq) was suspended in THF (150 mL), protected with nitrogen, cooled to 0-10° C., and potassium tert-butoxide (4.59 g, 40.8 mmol) was added, the temperature was controlled within 20° C. The reaction solution was stirred for 20 minutes after addition was completed (the reaction liquid turned into brown-red). The temperature was cooled to −50° C., and ethyl 5-chloro-1-(2,6-difluorobenzyl)-4-formyl-1H-pyrazole-3-carboxylate (3.2 g, 9.73 mmol) was dissolved in THF (100 mL), the reaction solution was dropped into it, and the temperature was controlled within −50° C. After the addition was completed, the temperature was naturally raised to room temperature and the reaction was proceeded for 2 hours (the reaction solution was yellow suspension). The reaction solution was cooled to 0-10° C., with saturated ammonium chloride (50 mL) added, stirred for 10 minutes, separated with liquid separation extraction, the aqueous phase was extracted with ethyl acetate (300 mL×2). The organic phases were combined, washed with saturated sodium chloride (50 mL) and dried over anhydrous sodium sulfate. The organic phases were evaporated to dryness and purified by column chromatography (EA/PE=0%-30%) to obtain the white solid product ethyl 5-chloro-1-(2,6-difluorobenzyl)-4-(2-methoxyvinyl)-1H-pyrazole-3-carboxylate (2.8 g, yield: 82%). ES-API: [M+H]⁺=357.25.
Step 5: ethyl 5-chloro-1-(2,6-difluorobenzyl)-4-(2-methoxyvinyl)-1H-pyrazole-3-carboxylate (300 mg, 0.84 mmol) was dissolved in THF (5 ml), 6N HCl (1 ml) was added dropwise slowly, the solution became colorless and transparent. Under stirring, the reaction was proceeded at 50° C. for 2 hours. 10 ml of pure water was added to the reaction bottle, 30 ml of ethyl acetate was added for extraction three times. The organic phases were combined, dried over anhydrous sodium sulfate, spin-dried to obtain the ethyl 5-chloro-1-(2,6-difluorobenzyl)-4-(2-oxoethyl)-1H-pyrazole-3-carboxylate (300 mg, crude product), the crude product was directly used in the next reaction. ES-API: [M+H]⁺=342.06.
Step 6: ethyl 5-chloro-1-(2,6-difluorobenzyl)-4-(2-oxoethyl)-1H-pyrazole-3-carboxylate (300 mg, 0.84 mmol) and 1,1-difluoropropyl-2-amine (95 mg, 1.0 mmol) were dissolved in methanol (10 ml), boranemethylpyridine complex (135 mg, 1.26 mmol) was added, and the reaction solution was stirred at room temperature, the reaction was proceeded for 16 hours. 10 ml of water was added to the reaction solution, 50 ml of ethyl acetate was added and the reaction solution was extracted three times. The organic phase was washed with 10 ml of saturated brine, dried with anhydrous sodium sulfate, evaporated to dryness, and purified by column chromatography (EA/PE=0%-35%) to obtain the white solid product that was a white crystal of ethyl 5-chloro-1-(2,6-difluorobenzyl)-4-(2-((1,1-difluoroprop-2-yl)amino)ethyl)-1H-pyrazole-3-carboxylate (354 mg, yield: 100%). ES-API: [M+H]⁺=422.12.
Step 7: ethyl 5-chloro-1-(2,6-difluorobenzyl)-4-(2-((1,1-difluoroprop-2-yl)amino)ethyl)-1H-pyrazole-3-carboxylate (354 mg, 0.84 mmol) was dissolved in 10 ml tetrahydrofuran and 1 ml methanol, evenly stirred. When the solution was colorless and transparent, 1N LiOH solution (2.5 mL) was added to the reaction solution and stirred at room temperature for 2 hours. The reaction was monitored by LCMS. After the reaction was completed, 1N HCl was used to adjust the reaction solvent to weak acidity, the solution changes from colorless and clear to white turbid. The solution was spin-dried to obtain 5-chloro-1-(2,6-difluorobenzyl)-4-(2-((1,1-difluoroprop-2-yl)amino)ethyl)-1H-pyrazole-3-carboxylic acid (500 mg, crude product). ES-API: [M+H]⁺=393.69.
Step 8: 5-chloro-1-(2,6-difluorobenzyl)-4-(2-((1,1-difluoroprop-2-yl)amino)ethyl)-1H-pyrazole-3-carboxylic acid (80 mg, 0.2 mmol) was dissolved in DCM (15 ml), DIEA (78 mg, 0.6 mmol) and HATU (84 mg, 0.22 mmol) were added, the reaction solution changed from a light yellow clear state to some white solids was precipitated. The reaction solution was stirred at room temperature of 25° C. for 1 hour and the reaction was monitored by LCMS. After the reaction was completed, the reaction solution was poured into 50 ml of water, extracted with ethyl acetate (50 mL×3), washed with saturated brine (50 mL×1). The organic phase was dried with anhydrous sodium sulfate, concentrated, and the crude product was separated by high performance liquid chromatography (column: Ultimate XB-C18, 50*250 mm, 10 um; A: purified water B: pure acetonitrile, flow rate: 80 ml/min, wavelength: 210 nm), to obtain 3-chloro-2-(2,6-difluorobenzyl)-6-(1,1-difluoroprop-2-yl)-2,4,5,6-tetrahydro-pyrazolo[3,4-c]pyridin-7-one (Z9, 18.6 mg, yield: 24.8%).¹H NMR (400 MHz, CDC13) δ 7.36-7.25 (m, 2H), 6.90 (t, J=8.0 Hz, 2H), 5.88 (t, J=2.6 Hz, 1H), 5.46 (s, 2H), 5.06-4.92 (m, 1H), 3.57 (t, J=6.5 Hz, 2H), 2.73 (q, J=6.4 Hz, 2H), 1.33 (d, J=7.2 Hz, 4H). ES-API: [M+H]⁺=375.08
Step 9: The compound (29) 3-chloro-2-(2,6-difluorobenzyl)-6-(1,1-difluoroprop-2-yl)-2,4,5,6-tetrahydro-7H-pyrazolo[3,4-c]pyridin-7-one (45 mg) obtained above was chiral separated (co-solvent: n-hexane: ethanol: diethanolamine=70:30:0.2)); column: AS-H (4.6*100 mm 5 um); flow rate: 0.5 ml/min; column temperature: 25° C.) to obtain two isomeric compounds. The structure of one compound (retention time: 2.566 min) arbitrarily assigned as (S)-3-chloro-2-(2,6-difluorobenzyl)-6-(1,1-difluoroprop-2-yl)-2,4,5,6-tetrahydro-7H-pyrazolo[3,4-c]pyridin-7-one (Z9-1, 18 mg, purity: 100%, ee value: 100%). ES-API: [M+H]⁺=376.0. The structure of another compound (retention time: 2.832 min) arbitrarily assigned as (R)-3-chloro-2-(2,6-difluorobenzyl)-6-(1,1-difluoroprop-2-yl)-2,4,5,6-tetrahydro-7H-pyrazolo[3,4-c]pyridin-7-one (Z9-2, 19 mg, purity: 100%, ee value: 100%). ES-API: [M+H]⁺=376.0.

### Example 10: Preparation of 3-chloro-2-(2,6-difluorobenzyl)-6-(1-(difluoromethyl)cyclopropyl)-2,4,5,6-tetrahydro-7H-pyrazolo[3,4-c]pyridin-7-one (Z10)

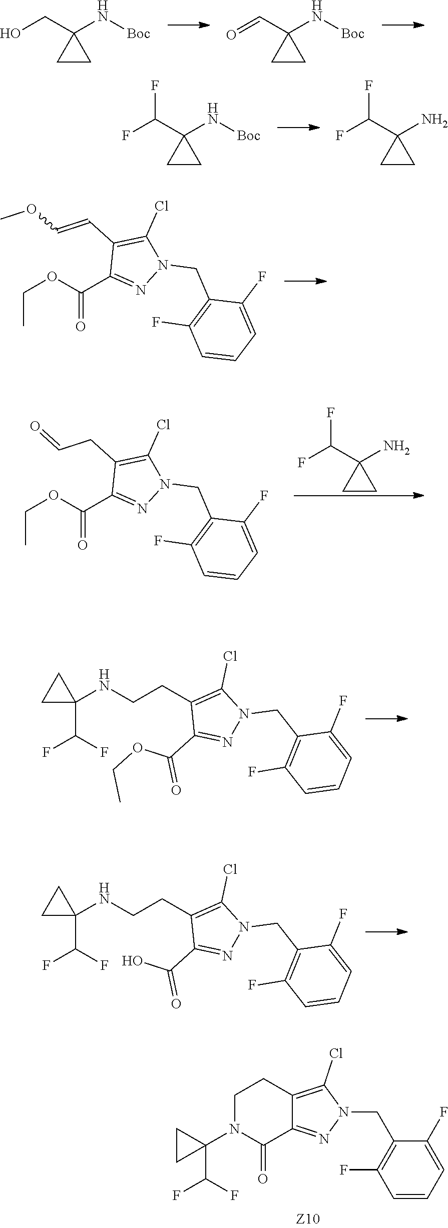 [0258] Step 1: 1-(hydroxymethyl)cyclopropyl)carbamic acid tert-butyl ester (5 g, 26.74 mmol) was dissolved in dichloromethane (10 mL), Dess-Martin periodinane (DMP) (17 g, 40.11 mmol) was added, and the reaction was proceeded at room temperature for 16 hours. The reaction solution was washed with sodium sulfite, sodium bicarbonate solution, and saturated brine in sequence, dried over anhydrous sodium sulfate, concentrated under reduced pressure, and purified by column chromatography (petroleum ether/ethyl acetate=5/1) to obtain (1-formylcyclopropyl)carbamic acid tert-butyl ester (2.1 g, yield: 42.4%). ES-API: [M+H]⁺=186.1.
Step 2: (1-formylcyclopropyl)carbamic acid tert-butyl ester (2.1 g, 11.35 mmol) was dissolved in dichloromethane (20 mL), diethylamino sulfur trifluoride (5.49 g, 34.05 mmol) was added, the reaction was proceeded at 20° C. for 4 hours. The reaction was quenched with ice water. The organic phase was washed with sodium bicarbonate solution and saturated brine, dried over anhydrous sodium sulfate, concentrated under reduced pressure, and performed column chromatography (petroleum ether/ethyl acetate=5/1) to obtain (1-(difluoromethyl)cyclopropyl)carbamic acid tert-butyl ester (170 mg, yield: 7.23%). ES-API: [M+H]⁺=208.1.
Step 3: 1-(difluoromethyl)cyclopropyl)carbamic acid tert-butyl ester (170 mg, 0.821 mmol) was dissolved in dichloromethane (1 mL) and trifluoroacetic acid (1 mL), and the reaction was proceeded at room temperature for 1 hour. The solvent was concentrated to dryness under reduced pressure. The pH of the reaction solution was adjusted to 12 with 1N sodium hydroxide solution, the reaction solution was extracted with dichloromethane. The organic phase was washed with saturated brine, dried over anhydrous sodium sulfate, and concentrated under reduced pressure to obtain 1-(difluoromethyl)cyclopropylamine. (50 mg, yield: 56.9%). ES-API: [M+H]⁺=108.1.
Step 4: ethyl 5-chloro-1-(2,6-difluorobenzyl)-4-(2-methoxyvinyl)-1H-pyrazole-3-carboxylate (200 mg, 0.562 mmol) was dissolved in 5N hydrochloric acid (2 mL) and tetrahydrofuran (2 mL), and reaction was proceeded at 50° C. for 1 hour. The reaction solution was cooled to room temperature, water was added. The reaction solution was extracted with ethyl acetate. The organic phase was washed with saturated brine, dried over anhydrous sodium sulfate, and concentrated under reduced pressure to obtain ethyl 5-chloro-1-(2,6-difluorobenzyl)-4-(2-oxoethyl)-1H-pyrazole-3-carboxylate (200 mg, crude product). ES-API: [M+H]⁺=343.1.
Step 5: ethyl 5-chloro-1-(2,6-difluorobenzyl)-4-(2-oxoethyl)-1H-pyrazole-3-carboxylate (200 mg, 0.467 mmol) was dissolved in methanol (2 mL), 1-(difluoromethyl)cyclopropylamine (50 mg, 0.467 mmol) and 2-methylpyridine borane complex (75 mg, 0.7 mmol) was added, and the reaction was proceeded at 25° C. for 5 h. The reaction solution was quenched by adding water, extracted with dichloromethane. The organic phase was washed with saturated brine, dried over anhydrous sodium sulfate, concentrated under reduced pressure, and purified by column chromatography (petroleum ether/ethyl acetate=3/1) to obtain ethyl 5-chloro-1-(2,6-difluorobenzyl)-4-(2-((1-(difluoromethyl)cyclopropyl)amino)ethyl)-1H-pyrazole-3-carboxylate (40 mg, yield: 19.8%). ES-API: [M+H]⁺=434.1.
Step 6: ethyl 5-chloro-1-(2,6-difluorobenzyl)-4-(2-((1-(difluoromethyl)cyclopropyl)amino)ethyl)-1H-pyrazole-3-carboxylate (40 mg, 0.092 mmol) was dissolved in tetrahydrofuran/water (1 mL/1 mL), lithium hydroxide monohydrate (8 mg, 0.185 mmol) was added, heated to 40° C. and the reaction was proceeded for 1 hour. The reaction solution was cooled to room temperature, adjusted to pH about 5 with 1N HCl, and concentrated under reduced pressure to obtain 5-chloro-1-(2,6-difluorobenzyl)-4-(2-((1-(difluoromethyl)cyclopropyl)amino)ethyl)-1H-pyrazole-3-carboxylic acid (70 mg, crude product). ES-API: [M+H]⁺=406.1.
Step 7: 5-chloro-1-(2,6-difluorobenzyl)-4-(2-((1-(difluoromethyl)cyclopropyl)amino)ethyl)-1H-pyrazole-3-carboxylic acid (70 mg, 0.173 mmol), N,N-diisopropylethylamine (56 mg, 0.433 mmol), O-(7-azabenzotriazol-1-yl)-N,N,N′,N′-tetramethyluronium hexafluorophosphate (79 mg, 0.207 mmol) was dissolved in N,N-dimethylformamide (2 mL) and reacted at 200° C. for 1 hour. The reaction solution was washed with 0.5N dilute hydrochloric acid, washed with saturated brine, dried over anhydrous sodium sulfate, spin-dried to dry the solvent, and purified by thin layer chromatography (petroleum ether/ethyl acetate=2/1) to obtain 3-chloro-2-(2,6-difluorobenzyl)-6-(1-(difluoromethyl)cyclopropyl)-2,4,5,6-tetrahydro-7H-pyrazolo[3,4-c]pyridin-7-one (Z10, 4.1 mg, yield: 6.12%). ¹H NMR (400 MHz, CDCl₃) 87.31 (q, J=8.2 Hz, 1H), 6.90 (t, J=7.9 Hz, 2H), 5.44 (s, 2H), 3.69 (s, 2H), 2.70 (t, J=6.4 Hz, 2H), 0.98 (s, 2H). ES-API: [M+H]⁺=388.1.

### Example 11: Preparation of 3-chloro-2-(2,6-difluorobenzyl)-6-((1-fluorocyclopropyl)methyl)-2,4,5,6-tetrahydro-7H-pyrazolo[3,4-c]pyridin-7-one(Z11)

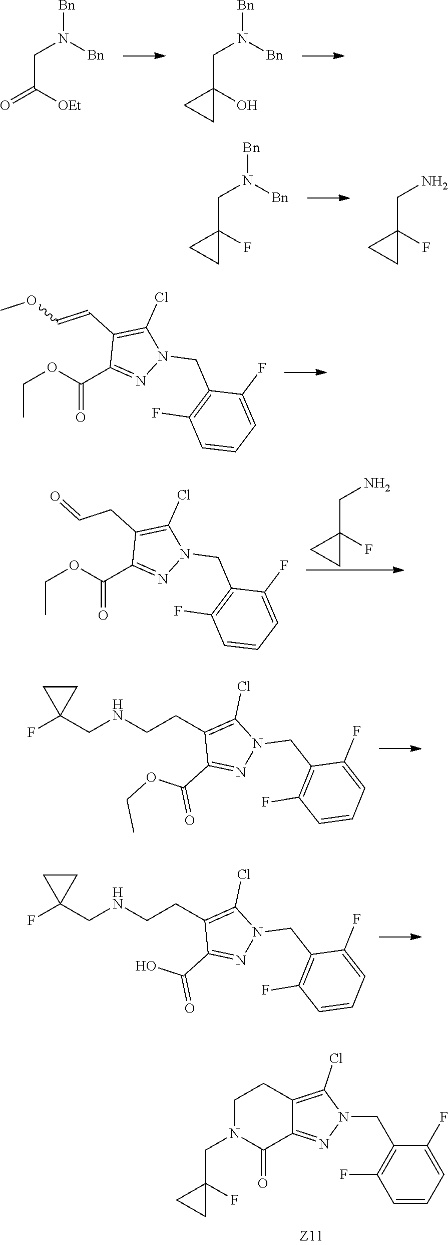 [0266] Step 1: 2-(dibenzylamino)ethyl acetate (6 g, 21.2 mmol) was dissolved in 30 mL tetrahydrofuran under nitrogen protection, tetraisopropyl titanate (1.46 g, 5.14 mmol) was added, and the temperature was controlled at 8˜12° C., 3 mol/L ethyl magnesium bromide solution (21.2 mL, 63.6 mmol) was added, and stirred at room temperature for 16 hours. The reaction solution was quenched with saturated ammonium chloride solution and filtered. The filter cake was washed with ethyl acetate (150 mL×2). The organic phase was washed with sodium bicarbonate (100 mL). The organic phases were combined, dried over anhydrous sodium sulfate and concentrated. The crude product was purified by column chromatography (petroleum ether/ethyl acetate=5/1) to obtain 3.2 g of colorless oily substance 1-((dibenzylamino)methyl)cyclopropanol. Yield: 53.8%, ES-API: [M+H]⁺=268.1.
Step 2: 1-((dibenzylamino)methyl)cyclopropanol (2 g, 7.4 mmol) was added to 40 mL toluene, the temperature was controlled at 8˜12° C., and diethylamine sulfur trifluoride (2.38 g, 14.8 mmol) was added to the reaction solution and stirred at room temperature for 16 hours. Ethyl acetate (100 mL) was added to the reaction solution, washed twice with saturated sodium bicarbonate solution (20 mL×2), and twice with water (20 mL×2). The organic phases were combined, dried over anhydrous sodium sulfate, and concentrated. The crude product was purified by column chromatography (petroleum ether/ethyl acetate=10/1) to obtain 1 g of colorless oily substance N,N-dibenzyl-1-(1-fluorocyclopropyl)methanamine. Yield: 50%, ES-API: [M+H]⁺=270.1.
Step 3: N,N-dibenzyl-1-(1-fluorocyclopropyl)methanamine (500 mg, 1.85 mmol) was dissolved in methanol (20 mL), 10% palladium on carbon (150 mg) was added, and stirred at room temperature for 16 hours in hydrogen atmosphere. The reaction solution was filtered, and the filtrate was concentrated to obtain crude (1-fluorocyclopropyl)methylamine, which was directly used in the next reaction. Yield: 100%. ES-API: [M+H]⁺=90.1.
Step 4: ethyl 5-chloro-1-(2,6-difluorobenzyl)-4-(2-methoxyvinyl)-1H-pyrazole-3-carboxylate (150 mg, 0.42 mmol) was dissolved in THF (5 ml), 6N HCl (1 ml) was added dropwise slowly, the solution became colorless and transparent. The reaction solution was stirred at 50° C. for 2 hours. 10 ml of pure water was added to the reaction bottle, 30 ml of ethyl acetate was added for extraction three times, the organic phases was combined, dried over anhydrous sodium sulfate and spin-dried to obtain ethyl 5-chloro-1-(2,6-difluorobenzyl)-4-(2-oxyethyl)-1H-pyrazole-3-carboxylate (150 mg, crude product), which was directly used next reaction. ES-API: [M+H]⁺=343.06.
Step 5: ethyl 5-chloro-1-(2,6-difluorobenzyl)-4-(2-oxyethyl)-1H-pyrazole-3-carboxylate (150 mg, 0.42 mmol) and (1-fluorocyclopropyl)methanamine (56 mg, 0.63 mmol) was dissolved in methanol (10 ml), boranemethylpyridine complex (89 mg, 0.84 mmol) was added, and the reaction solution was stirred at room temperature for 1 hour. 10 ml of water was added to the reaction solution, 50 ml of ethyl acetate was added and extracted three times. The organic phase was washed with 10 ml of saturated brine, dried over anhydrous sodium sulfate, evaporated to dryness, and purified by column chromatography (EA/PE=10%-50%) to obtain ethyl 5-chloro-1-(2,6-difluorobenzyl)-4-(2-(((1-fluorocyclopropyl)methyl)amino)ethyl)-1H-pyrazole-3-carboxylate (100 mg, yield: 57.4%). Brown oily substance. ES-API: [M+H]⁺=416.1.
Step 6: ethyl 5-chloro-1-(2,6-difluorobenzyl)-4-(2-(((1-fluorocyclopropyl)methyl)amino)ethyl)-1H-pyrazole-3-carboxylate (100 mg, 0.24 mmol) was dissolved in 5 ml tetrahydrofuran and 1 ml methanol, stirred evenly. The solution was colorless and transparent, 1N LiOH solution (2.5 mL) was added to the reaction solution. Under stirring, the reaction was proceeded for 16 hours at room temperature. LCMS monitored the reaction and the reaction was completed. 1N HCl was used to adjust the reaction solvent to weak acidity. The solution changed from colorless and clear to white turbidity and was spin-dried to obtain crude product of 5-chloro-1-(2,6-difluorobenzyl)-4-(2-(((1-fluorocyclopropyl)methyl)amino)ethyl)-1H-pyrazole-3-carboxylic acid (100 mg, yield: 100%). ES-API: [M+H]⁺=388.1.
Step 7: 5-chloro-1-(2,6-difluorobenzyl)-4-(2-(((1-fluorocyclopropyl)methyl)amino)ethyl)-1H-pyrazole-3-carboxylic acid (100 mg, 0.24 mmol) was dissolved in DCM (10 ml), DIEA (93 mg, 0.72 mmol) and HATU (100 mg, 0.26 mmol) were added. The reaction was stirred at room temperature of 25° C. for 1 hour, monitored by LCMS. After the reaction was completed, the reaction solution was poured into 50 ml of dichloromethane, washed twice with 10 ml of water. The organic phase was dried with anhydrous sodium sulfate, concentrated. The crude product was purified by high performance liquid chromatography (column: Ultimate XB-C18, 50*250 mm, 10 um; A: purified water B: pure acetonitrile, flow rate: 80 ml/min, wavelength: 210 nm) to obtain 3-chloro-2-(2,6-difluorobenzyl)-6-((1-fluorocyclopropyl)methyl)-2,4,5,6-tetrahydro-7H-pyrazolo[3,4-c]pyridin-7-one (Z11, 18.3 mg, yield: 20.6%).¹H NMR (400 MHz, CDCl³) δ 7.30 (d, J=7.4 Hz, 1H), 6.89 (t, J=8.0 Hz, 2H), 5.44 (s, 2H), 4.49 (d, J=49.1 Hz, 2H), 3.74 (t, J=6.5 Hz, 2H), 2.67 (t, J=6.5 Hz, 2H), 1.03 (s, 4H). ES-API: [M+H]⁺=370.1.

### Example 12: Preparation of 3-chloro-6-(1,1-difluoropropan-2-yl)-2-(2-(trifluoromethoxy)benzyl)-2,4,5,6-tetrahydro-7H-pyrazolo[3,4-c]pyridin-7-one (Z12)

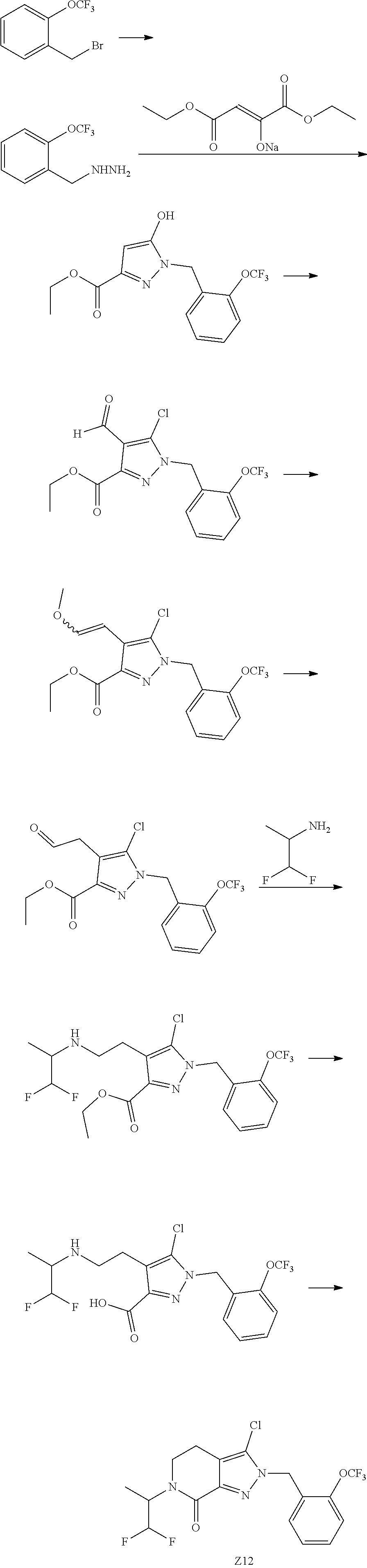 [0274] Step 1: 2-(trifluoromethoxy)benzyl bromide (5 g, 19.6 mmol) was dissolved in 150 mL ethanol, added dropwise to hydrazine hydrate (30 g, 588 mmol) at 60° C. Under stirring, the reaction was proceeded at 60° C. for 3 hours. The reaction solution was concentrated under reduced pressure to remove excess hydrazine hydrate, and performed column chromatography (dichloromethane/methanol=1%˜10%) to obtain 2-(trifluoromethoxy)benzylhydrazine (2.8 g, yield: 69%). ES-API: [M+H]+=207.1.
Step 2: diethyl oxaloacetate sodium salt (2.13 g, 10.10 mmol) was dissolved in 40 mL acetic acid, 2-(trifluoromethoxy)benzylhydrazine (2.8 g, 13.5 mmol) was added. Under stirring, the reaction was proceeded at 100° C. for 16 hours. The reaction solution was concentrated under reduced pressure to remove acetic acid, extracted with ethyl acetate. The crude product was purified by column chromatography (petroleum ether/ethyl acetate=10/1-1/1) to obtain ethyl 5-hydroxy-1-(2-(trifluoromethoxy)benzyl)-1H-pyrazole-3-carboxylate (2.67 g, yield: 60%). ES-API: [M+H]⁺=331.0.
Step 3: ethyl 5-hydroxy-1-(2-(trifluoromethoxy)benzyl)-1H-pyrazole-3-carboxylate (2.67 g, 8.06 mmol) was dissolved in phosphorus oxychloride (30 mL), DMF (3 mL) was added dropwise. The reaction solution was heated to 90° C. and reacted for 16 hours. The reaction solution was cooled to room temperature. Phosphorus oxychloride in the reaction solution was removed by rotary evaporation. The residue was dissolved with dichloromethane and added dropwise into ice water. The solution was extracted with dichloromethane, dried with anhydrous sodium sulfate. The solvent was spin-dried, and the crude product was purified by column chromatography (petroleum ether/ethyl acetate=10/1-5/1) to obtain ethyl 5-chloro-4-formyl-1-(2-trifluoromethoxy)benzyl)-1H-pyrazole-3-carboxylate (1.75 g, Y: 58%). ES-API: [M+H]⁺=377.0.
Step 4: Under nitrogen protection, methoxymethyltriphenylphosphine chloride (3.35 g, 9.76 mmol) and potassium tert-butoxide (987 mg, 8.82 mmol) were dissolved in anhydrous tetrahydrofuran (10 mL). The reaction solution was cooled to −30° C., and the solution of ethyl 5-chloro-4-formyl-1-(2-trifluoromethoxy)benzyl)-1H-pyrazole-3-carboxylate (800 mg, 2.1 mmol) was added dropwise into tetrahydrofuran (10 mL). The reaction solution was stirred and reacted for 1 hour, slowly raised to room temperature, quenched with saturated ammonium chloride solution, extracted with ethyl acetate. The solvent was removed by rotary evaporation. The crude product was purified by column chromatography (petroleum ether/ethyl acetate=10/1-5/1) to obtain ethyl 5-chloro-4-(2-methoxyvinyl)-1-(2-trifluoromethoxy)benzyl)-1H-pyrazole-3-carboxylate (100 mg, yield: 12%). ES-API: [M+H]⁺=405.0.
Step 5: ethyl 5-chloro-4-(2-methoxyvinyl)-1-(2-trifluoromethoxy)benzyl)-1H-pyrazole-3-carboxylate (230 mg, 0.57 mmol) was dissolved in tetrahydrofuran (5 mL), and hydrochloric acid (5N, 1 mL) was added. The reaction solution was stirred and the reaction was proceeded at 60° C. for 1 hour. The reaction solution was cooled to room temperature, extracted with ethyl acetate, dried over anhydrous sodium sulfate, and rotary evaporated to remove the solvent to obtain ethyl 5-chloro-4-(2-oxoethyl)-1-(2-trifluoromethoxy)benzyl)-1H-pyrazole-3-carboxylate (230 mg, crude product). ES-API: [M+H]⁺=391.0.
Step 6: ethyl 5-chloro-4-(2-oxoethyl)-1-(2-trifluoromethoxy)benzyl)-1H-pyrazole-3-carboxylate (230 mg, crude product) was dissolved in methanol (3 mL), borane-2-methylpyridine complex (82 mg, 0.77 mmol) and 1,1-difluoropropan-2-amine (67 mg, 0.71 mmol) were added. The reaction solution was stirred at room temperature for 16 hours. The reaction solution was extracted with dichloromethane, dried over anhydrous sodium sulfate, and the solvent was removed by rotary evaporation to obtain 5-chloro-4-(2-((1,1-difluoropropan-2-yl)amino)ethyl)-1-(2-(trifluoromethoxy)benzyl)-1H-pyrazole-3-carboxylatic acid ethyl ester (270 mg, crude product). ES-API: [M+H]+=470.1.
Step 7: 5-chloro-4-(2-((1,1-difluoropropan-2-yl)amino)ethyl)-1-(2-(trifluoromethoxy)benzyl)-1H-pyrazole-3-carboxylatic acid ethyl ester (270 mg, crude product) was dissolved in methanol-water (1 mL/1 mL). Lithium hydroxide (27 mg, 0.63 mmol) was added. The reaction was proceeded at room temperature for 1 hour. The reaction solution was adjusted to pH=5-6 with hydrochloric acid (2N), extracted with dichloromethane. The solvent was removed by rotary evaporation to obtain 5-chloro-4-(2-((1,1-difluoropropan-2-yl)amino)ethyl)-1-(2-(trifluoromethoxy)benzyl)-1H-pyrazole-3-carboxylic acid (250 mg, crude product). ES-API: [M+H]⁺=442.1.
Step 8: 5-chloro-4-(2-((1,1-difluoropropan-2-yl)amino)ethyl)-1-(2-(trifluoromethoxy)benzyl)-1H-pyrazole-3-carboxylic acid (25 mg, crude product) was dissolved in dichloromethane (5 mL), N,N-diisopropylethylamine (108 mg, 0.84 mmol) and HATU (258 mg, 0.68 mmol) was added. The reaction solution was stirred at room temperature and reacted for 1 hour. The reaction solution was extracted with dichloromethane, dried over anhydrous sodium sulfate, and the solvent was removed by rotary evaporation. The crude product was purified by column chromatography (petroleum ether/ethyl acetate=1/1) to obtain white solid 3-chloro-6-(1,1-difluoropropan-2-yl)-2-(2-(trifluoromethoxy)benzyl)-2,4,5,6-tetrahydro-7H-pyrazolo[3,4-c]pyridin-7-one, (Z12, 13.9 mg, yield: 5%).¹H NMR (400 MHz, CDCl3) δ7.34 (t, J=7.9 Hz, 1H), 7.28 (s, 1H), 7.22 (t, J=7.6 Hz, 1H), 7.02 (d, J=7.7 Hz, 1H), 6.15 -5.68 (m, 1H), 5.50 (s, 2H), 5.04 (dd, J=18.6, 8.7 Hz, 1H), 3.62 (t, J=6.6 Hz, 2H), 2.76 (q, J=6.7 Hz, 2H), 1.36 (d, J=7.2 Hz, 3H). ES-API: [M+H]+=424.1.

### Example 13: Preparation of 3-chloro-6-(1-cyclopropyl-2,2-difluoroethyl)-2-(2,6-difluorobenzyl)-2,4,5,6-tetrahydro-7H-pyrazolo[3,4-c]pyridin-7-one (Z13)

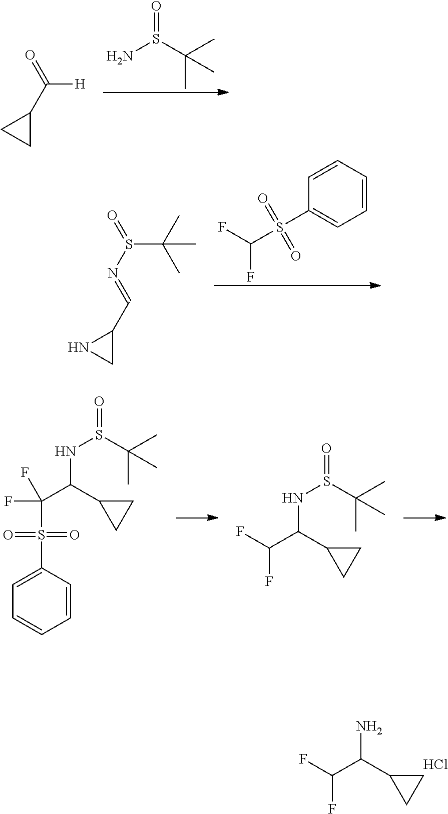 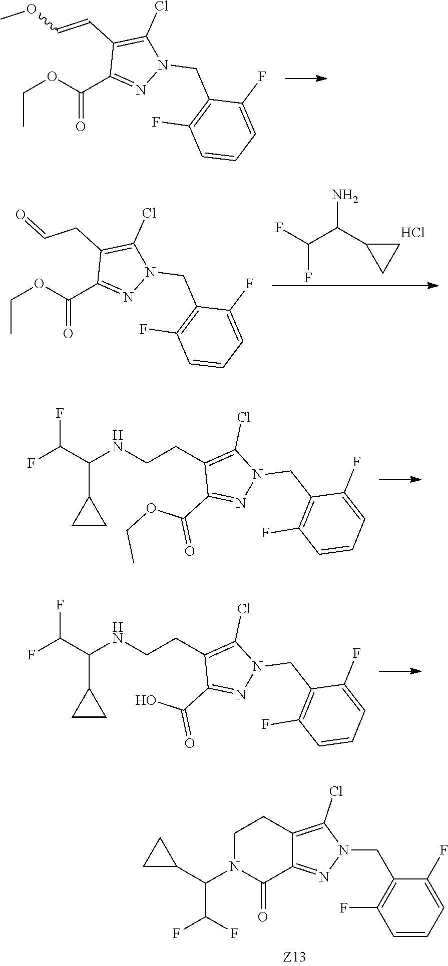 [0283] Step 1: cyclopropanecarboxaldehyde (5.67 g, 81 mmol) and 2-methylpropane-2-sulfinamide (9.8 g, 81 mmol) was dissolved in tetrahydrofuran (60 mL), and tetraisopropyl titanate (46 g, 162 mmol) was added. The reaction was proceeded at room temperature for 16 hours. The reaction solution was quenched with saturated brine, filtered with suction, and the filtrate was extracted with ethyl acetate, dried over anhydrous sodium sulfate, concentrated under reduced pressure, and purified by column chromatography (petroleum ether/ethyl acetate=3/1) to obtain N-(cyclopropylmethylene)-2-methylpropane-2-sulfenamide (8.5 g, yield: 60.7%). ES-API: [M+H]⁺=174.1.
Step 2: N-(cyclopropylmethylene)-2-methylpropane-2-sulfenamide (8.5 g, 49.1 mmol), (difluoromethyl)sulfonyl)benzene (9 g, 46.8 mmol) was dissolved in tetrahydrofuran (90 mL) LiHMDS (94 mL) was added dropwise at −78° C., and the reaction was proceeded at −78° C. for 1 hour.

The reaction solution was quenched with 0.5 N dilute hydrochloric acid, extracted with ethyl acetate. The organic phase was washed with saturated brine, dried with anhydrous sodium sulfate, concentrated under reduced pressure, and purified by column chromatography (petroleum ether/ethyl acetate=3/1) to obtain N-(1-cyclopropyl-2,2-difluoro-2-(phenylsulfonyl)ethyl)-2-methylpropane-2-sulfinamide (13 g, yield: 76.1%). ES-API: [M+H]⁺=366.1.
Step 3: N-(1-cyclopropyl-2,2-difluoro-2-(phenylsulfonyl)ethyl)-2-methylpropane-2-sulfinamide (5 g, 13.7 mmol) was dissolved in DMF (30 mL), acetic acid (37.25 g, 618 mmol), sodium acetate (50.75 g, 618 mmol), water (10 mL), magnesium chips (8.23 g, 343 mmol) after treatment with dilute hydrochloric acid were added, and the reaction was proceeded at room temperature for 16 hours. The reaction solution was filtered with suction, quenched with 0.5N dilute hydrochloric acid, extracted with ethyl acetate. The organic phase was concentrated to dry solvent under reduced pressure, and purified by column chromatography (petroleum ether/ethyl acetate=3/1) to obtain N-(1-cyclopropyl)-2,2-difluoroethyl)-2-methylpropane-2-sulfinamide (2.2 g, yield: 42.8%). ES-API: [M+H]⁺=226.1.
Step 4: N-(1-cyclopropyl-2,2-difluoroethyl)-2-methylpropane-2-sulfinamide (1 g, 4.44 mmol) was dissolved in methanol (5 mL) and HCl/i-PrOAc (5 mL), the reaction was proceeded at 20° C. for 0.5 hour. The solvent was concentrated to dry, the product was dissolved in dichloromethane, the pH was adjusted to 12. The organic phase was washed with saturated brine, dried over anhydrous sodium sulfate, and concentrated under reduced pressure to obtain 1-cyclopropyl-2,2-difluoroethylamine (850 mg, crude product). ES-API: [M+H]⁺=122.1.
Step 5: ethyl 5-chloro-1-(2,6-difluorobenzyl)-4-(2-methoxyvinyl)-1H-pyrazole-3-carboxylate (30 mg, 0.084 mmol) was dissolved in 5N hydrochloric acid (2 mL) and tetrahydrofuran (2 mL), the reaction was proceeded at 50° C. for 1 hour. The reaction solution was cooled to room temperature, water was added, the reaction solution was extracted with ethyl acetate. The organic phase was washed with saturated brine, dried over anhydrous sodium sulfate, and concentrated under reduced pressure to obtain ethyl 5-chloro-1-(2,6-difluorobenzyl)-4-(2-oxoethyl)-1H-pyrazole-3-carboxylate (20 mg, yield: 69.7%). ES-API: [M+H]⁺=343.1.
Step 6: ethyl 5-chloro-1-(2,6-difluorobenzyl)-4-(2-oxoethyl)-1H-pyrazole-3-carboxylate (20 mg, 0.058 mmol) was dissolved in methanol (2 mL), 1-cyclopropyl-2,2-difluoroethylamine (7 mg, 0.058 mmol) and 2-methylpyridineborane complex (9 mg, 0.087 mmol) were added, and the reaction was proceeded at 25° C. for 16 hours. The reaction solution was quenched by adding water, extracted with dichloromethane, washed with saturated brine, dried over anhydrous sodium sulfate, concentrated under reduced pressure, and purified by column chromatography (petroleum ether/ethyl acetate=3/1) to obtain ethyl 5-chloro-4-(2-((1-cyclopropyl-2,2-difluoroethyl)amino)ethyl)-1-(2,6-difluorobenzyl)-1H-pyrazole-3-carboxylate (20 mg, yield: 77.2%). ES-API: [M+H]⁺=448.1.
Step 7: ethyl 5-chloro-4-(2-((1-cyclopropyl-2,2-difluoroethyl)amino)ethyl)-1-(2,6-difluorobenzyl)-1H-pyrazole-3-carboxylate (20 mg, 0.045 mmol) was dissolved in tetrahydrofuran/water (1 mL/1 mL), lithium hydroxide monohydrate (4 mg, 0.09 mmol) was added, heated to 40° C. and the reaction was proceeded for 1 hour. The reaction solution was cooled to room temperature, adjusted to pH around 5 with 1NHCl, concentrated under reduced pressure, 5-chloro-4-(2-((1-cyclopropyl-2,2-difluoroethyl)amino)ethyl)-1-(2,6-difluorobenzyl)-1H-pyrazole-3-carboxylic acid (30 mg, crude product). ES-API: [M+H]⁺=420.1.
Step 8: 5-chloro-4-(2-((1-cyclopropyl-2,2-difluoroethyl)amino)ethyl)-1-(2,6-difluorobenzyl)-1H-pyrazole-3-carboxylic acid (30 mg, 0.07 mmol), N,N-diisopropylethylamine (23 mg, 0.175 mmol), O-(7-azabenzotriazol-1-yl)-N,N,N′,N′-tetramethyluronium hexafluorophosphate (33 mg, 0.086 mmol) were dissolved in N,N-dimethylformamide (2 mL) and the reaction was proceeded at 20° C. for 1 hour. The reaction solution was washed with 0.5N dilute hydrochloric acid, washed with saturated brine, dried over anhydrous sodium sulfate, spin-dried to dry the solvent, and purified by thin layer chromatography (petroleum ether/ethyl acetate=2/1) to obtain 3-chloro-6-(1-cyclopropyl-2,2-difluoroethyl)-2-(2,6-difluorobenzyl)-2,4,5,6-tetrahydro-7H-pyrazolo[3,4-c]pyridin-7-one(Z13, 1.5 mg, yield: 5.36%). ¹H NMR (400 MHz, CDCl₃) δ 87.30 (s, 1H), 6.90 (t, J=8.0 Hz, 2H), 5.94 (s, 0H), 5.46 (s, 2H), 4.06 (d, J=12.5 Hz, 1H), 3.74 (q, J=7.2, 6.8 Hz, 2H), 2.77 (q, J=6.6 Hz, 2H), 1.21 (s, 1H), 0.79 (s, 1H), 0.68 -0.48 (m, 2H), 0.29 (s, 1H). ES-API: [M+H]⁺=402.1.

### Example 14: Preparation of 3-chloro-2-(3,5-difluorobenzyl)-6-(1,1-difluoropropan-2-yl)-2,4,5,6-tetrahydro-7H-pyrazolo[3,4-c]pyridin-7-one (Z14)

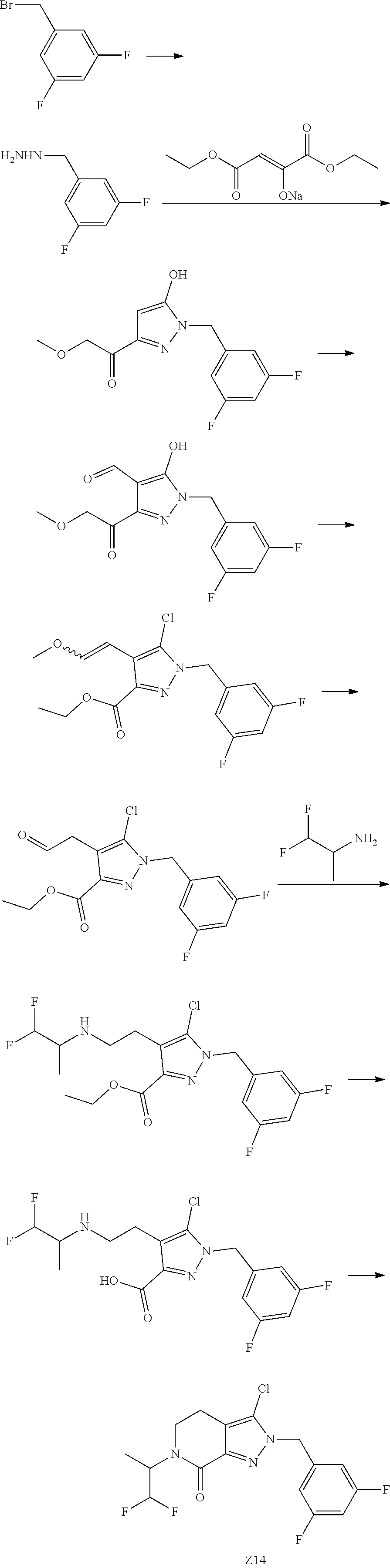 [0293] Step 1: A solution of 1-(bromomethyl)-3,5-difluorobenzene (8 g, 38.6 mmol) in ethanol (100 mL) was added dropwise to hydrazine hydrate (30 g, 600 mmol) at 60° C. After the addition was completed, the temperature was maintained and the reaction was continued for 2 hours. The reaction solution was concentrated under reduced pressure, and the crude product was subjected to column chromatography (dichloromethane/methanol=30/1-10/1) to obtain (3,5-difluorobenzyl)hydrazine (5.34 g, yield: 87%). ES-API: [M+H]⁺=159.1.
Step 2: diethyl oxaloacetate sodium salt (5.32 g, 25.3 mmol) was dissolved in acetic acid (50 mL), and (3,5-difluorobenzyl)hydrazine (5.34 g, 33.8 mmol) was added. The reaction was proceeded at 100° C. for 16 hours. The reaction solution was concentrated under reduced pressure, the residue was dissolved in ethyl acetate, washed with saturated sodium bicarbonate solution, and the organic phase was concentrated. The crude product was purified by column chromatography (petroleum ether/ethyl acetate=5/1-1/1) to obtain ethyl 1-(3,5-difluorobenzyl)-5-hydroxy-1H-pyrazole-3-carboxylate (3 g, yield: 44%). ES-API: [M+H]⁺=283.1.
Step 3: ethyl 1-(3,5-difluorobenzyl)-5-hydroxy-1H-pyrazole-3-carboxylate (3.0 g, 10.6 mmol) was dissolved in phosphorus oxychloride (30 mL), N,N-dimethylformamide (3 mL) was added. The reaction solution reacted at 100° C. for 16 hours. The reaction solution was concentrated under reduced pressure to remove phosphorus oxychloride. The residue was dissolved in dichloromethane, added to ice water dropwise, washed with sodium bicarbonate solution, and extracted with dichloromethane. The organic phase was concentrated under reduced pressure, and the crude product was purified by column chromatography (petroleum ether/ethyl acetate=15/1-8/1) to obtain ethyl 5-chloro-1-(3,5-difluorobenzyl)-4-formyl-1H-pyrazole-3-carboxylate (500 mg, yield: 14%). ES-API: [M+H]⁺=329.0.
Step 4: methoxymethyltriphenylphosphine chloride (1.43 g, 4.20 mmol) and potassium tert-butoxide (430 mg, 3.80 mmol) was suspended in tetrahydrofuran (20 mL), cooled to −30° C. under nitrogen protection, and a solution of ethyl 5-chloro-1-(3,5-difluorobenzyl)-4-formyl-1H-pyrazole-3-carboxylate (300 mg, 0.91 mmol) in tetrahydrofuran (5 mL) was added dropwise. After the addition was completed, the temperature was maintained and the reaction was continued for 30 minutes, then the temperature was slowly increased to room temperature. The reaction was quenched with ammonium chloride solution, extracted with ethyl acetate, concentrated under reduced pressure, and the crude product was purified by column chromatography (petroleum ether/ethyl acetate=10/1-8/1) to obtain ethyl 5-chloro-1-(3,5-difluorobenzyl)-4-(2-methoxyvinyl)-1H-pyrazole-3-carboxylate (278 mg, yield: 85%). ES-API: [M+H]⁺=357.1.
Step 5: ethyl 5-chloro-1-(3,5-difluorobenzyl)-4-(2-methoxyvinyl)-1H-pyrazole-3-carboxylate (100 mg, 0.28 mmol) was dissolved in tetrahydrofuran (5 mL) and hydrochloric acid (1 mL, 5N) was added. The reaction was proceeded at 60° C. for 30 minutes. The reaction solution was extracted with ethyl acetate, and concentrated under reduced pressure to obtain ethyl 5-chloro-1-(3,5-difluorobenzyl)-4-(2-oxyethyl)-1H-pyrazole-3-carboxylate (109 mg, crude product). ES-API: [M+H]⁺=343.1.
Step 6: ethyl 5-chloro-1-(3,5-difluorobenzyl)-4-(2-oxyethyl)-1H-pyrazole-3-carboxylate (109 mg, 0.32 mmol) and 1,1-difluoropropan-2-amine (46 mg, 0.32 mmol) were dissolved in methanol (5 mL). The solution was stirred for 10 minutes and borane-2-methylpyridine complex (51 mg, 0.48 mmol) was added. Under stirring, the reaction was proceeded at room temperature for 1 hour. The reaction solution was extracted with ethyl acetate and concentrated under reduced pressure to obtain ethyl 5-chloro-1-(3,5-difluorobenzyl)-4-(2-((1,1-difluoropropan-2-yl)amino)ethyl)-1H-pyrazole-3-carboxylate (110 mg, crude product). ES-API: [M+H]⁺=422.1.
Step 7: ethyl 5-chloro-1-(3,5-difluorobenzyl)-4-(2-((1,1-difluoropropan-2-yl)amino)ethyl)-1H-pyrazole-3-carboxylate (110 mg, 0.26 mmol) was dissolved in methanol/water (2 mL/1 mL), and lithium hydroxide monohydrate (16 mg, 0.39 mmol) was added. Under stirring, the reaction was proceeded at room temperature for 1 hour. The reaction solution was extracted with ethyl acetate, and the aqueous phases were combined. The pH of the aqueous phase was adjusted to 6-7 with dilute hydrochloric acid (2N), the aqueous phase was extracted again with ethyl acetate, and concentrated under reduced pressure to obtain 5-chloro-1-(3,5-difluorobenzyl)-4-(2-((1,1-difluoropropan-2-yl)amino)ethyl)-1H-pyrazole-3-carboxylic acid (110 mg, crude product). ES-API: [M+H]⁺=394.1.
Step 8: 5-chloro-1-(3,5-difluorobenzyl)-4-(2-((1,1-difluoropropan-2-yl)amino)ethyl)-1H-pyrazole-3-carboxylic acid (110 mg, 0.28 mmol) was dissolved in dichloromethane (3 mL), and N,N-diisopropylethylamine (0.5 mL) and HATU (1272 mg, 0.34 mmol) were added. Under stirring, the reaction was proceeded at room temperature for 2 hours. The reaction solution was extracted with dichloromethane, concentrated under reduced pressure, and the crude product was purified by thin layer chromatography (petroleum ether/ethyl acetate=1/1) to obtain 3-chloro-2-(3,5-difluorobenzyl)-6-(1,1-difluoropropan-2-yl)-2,4,5,6-tetrahydro-7H-pyrazolo[3,4-c]pyridin-7-one (Z14, 3.3 mg, yield: 16%). ¹H NMR (400 MHz, CDCl₃) δ 6.76 (dd, J=19.8, 8.1 Hz, 3H), 5.90 (t, J=56.0 Hz, 1H), 5.37 (s, 2H), 5.05 (dt, J=18.2, 8.2 Hz, 1H), 3.61 (t, J=6.6 Hz, 2H), 2.76 (q, J=6.9 Hz, 2H), 1.36 (d, J=7.2 Hz, 3H). ES-API: [M+H]⁺=376.1.

### Example 15: Preparation of 3-chloro-2-(2,6-difluorobenzyl)-6-(1,1-difluorobutan-2-yl)-2,4,5,6-tetrahydro-7H-pyrazolo[3,4-c]pyridin-7-one (Z15)

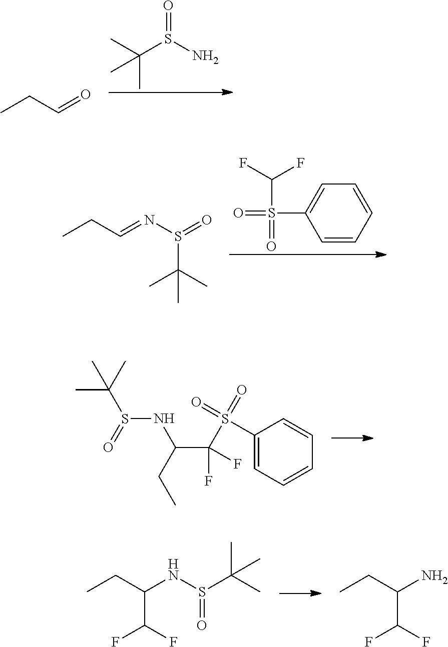 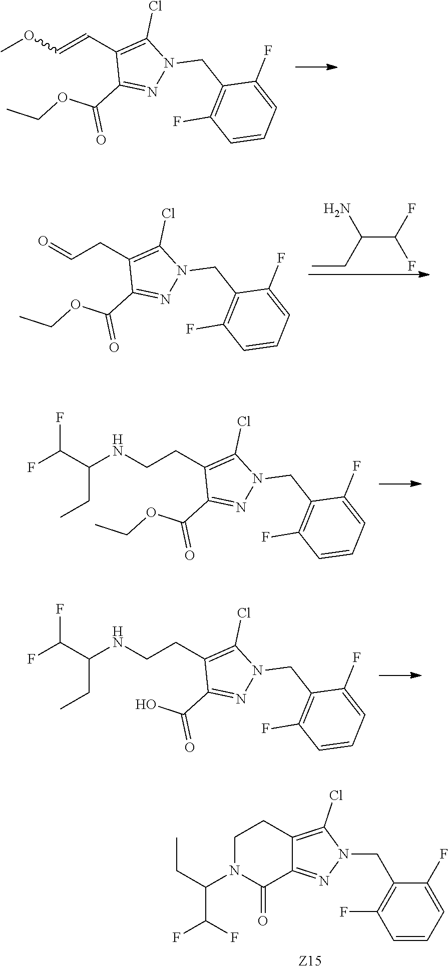 [0302] Step 1: propionaldehyde (1 g, 17.2 mmol) and (E)-2-methyl-N-propylenepropane-2-sulfenamide (2.3 g, 18.9 mmol) were dissolved in tetrahydrofuran (30 mL), and Ti(OEt)₄(14 g, 51.6 mmol) was added. Under stirring, the reaction was proceeded at room temperature for 16 hours. Water was added to the reaction solution, stirred for another 15 minutes, diatomaceous earth was added and filtered, and washed with ethyl acetate. The filtrate was concentrated under reduced pressure, to obtain (E)-2-methyl-N-propylenepropane-2-sulfenamide(3 g, crude product). ES-API: [M+H]⁺=162.1.
Step 2: (E)-2-methyl-N-propylenepropane-2-sulfenamide (2.00 g, 12.4 mmol) and ((difluoromethyl)sulfonyl)benzene (2.86 g, 15 mmol) were dissolved in anhydrous tetrahydrofuran (20 mL). The reaction solution was cooled to-70° C., and LiHMDS (15 mL, 15 mmol, 1 N in THF) was added dropwise. After the addition was completed, the temperature was maintained for 30 minutes and then slowly increased to room temperature. The reaction solution was quenched with dilute hydrochloric acid (1N), extracted with ethyl acetate, and concentrated under reduced pressure. The crude product was purified by column chromatography (petroleum ether/ethyl acetate=5/1-3/1) to obtain (E)-N-(1,1-difluoro-1-(phenylsulfonyl)butan-2-yl)-2-methylpropane-2-sulfinamide (973 mg, yield: 15%). ES-API: [M+H]⁺=354.1.
Step 3: (E)-N-(1,1-difluoro-1-(phenylsulfonyl)butan-2-yl)-2-methylpropane-2-sulfinamide (973 mg, 2.76 mmol) was dissolved in N,N-dimethylformamide/water (12 mL/4 mL), acetic acid (7.4 g, 124 mmol) and sodium acetate (10.18 g, 124 mmol) were added. The reaction solution was cooled to 0° C., and magnesium strips which was washed with dilute hydrochloric acid (1.32 g, 55 mmol) were added in batches. The reaction was proceeded at room temperature for 72 hours. The reaction solution was extracted with ethyl acetate, concentrated under reduced pressure, and the crude product was purified by column chromatography (petroleum ether/ethyl acetate=8/1-3/1) to obtain N-(1,1-difluorobutan-2-yl)-2-methylpropane-2-sulfinamide (300 mg, crude product). ES-API: [M+H]⁺=214.1.
Step 4: N-(1,1-difluorobutan-2-yl)-2-methylpropane-2-sulfenamide (500 mg, 2.35 mmol) was dissolved in methanol (3 mL), dioxane hydrochloride solution (3 mL) was added. Under stirring, the reaction was proceeded at room temperature for 1 hour. The pH of the reaction solution was adjusted to 13-14 with sodium hydroxide solution (6N), the reaction solution was extracted with dichloromethane, and concentrated under reduced pressure to obtain 1,1-difluorobutane-2-amine (500 mg, crude product), which was directly used next reaction. ES-API: [M+H]⁺=110.1.
Step 5: ethyl 5-chloro-1-(2,6-difluorobenzyl)-4-(2-methoxyvinyl)-1H-pyrazole-3-carboxylate (35 mg, 0.098 mmol) was dissolved in tetrahydrofuran (2 mL) and hydrochloric acid (5N, 1 mL) was added. Under stirring, the reaction was proceeded at 50° C. for 1 hour. The reaction solution was extracted with ethyl acetate and concentrated under reduced pressure to obtain ethyl 5-chloro-1-(2,6-difluorobenzyl)-4-(2-oxyethyl)-1H-pyrazole-3-carboxylate (40 mg, crude product), which was directly used it for the next reaction. ES-API: [M+H]⁺=343.1.
Step 6: ethyl 5-chloro-1-(2,6-difluorobenzyl)-4-(2-oxyethyl)-1H-pyrazole-3-carboxylate was dissolved in methanol (2 mL), and 1,1-difluorobutane-2-amine (500 mg, crude product) was added. Under stirring, the reaction was proceeded at room temperature for 5 minutes, and borane-2-methylpyridine complex (76 mg, 0.728 mmol) was added. Under stirring, the reaction was proceeded at room temperature overnight, The reaction solution was extracted with ethyl acetate and concentrated under reduced pressure to obtain ethyl 5-chloro-1-(2,6-difluorobenzyl)-4-(2-((1,1-difluorobutan-2-yl)amino)ethyl)-1H-pyrazole-3-carboxylate (30 mg, crude product), the crude product was directly used in the next reaction. ES-API: [M+H]⁺=436.1.
Step 7: ethyl 5-chloro-1-(2,6-difluorobenzyl)-4-(2-((1,1-difluorobutan-2-yl)amino)ethyl)-1H-pyrazole-3-carboxylate (30 mg, crude product) was dissolved in methanol/water (1 mL/0.5 mL), and lithium hydroxide monohydrate (5 mg, 0.082 mmol) was added. Under stirring, the reaction solution was proceeded at room temperature for 1 hour. The reaction solution was extracted with ethyl acetate. The aqueous phases were combined, the pH of which was adjusted to 6-7 with dilute hydrochloric acid (2N), and concentrated under reduced pressure to obtain 5-chloro-1-(2,6-difluorobenzyl)-4-(2-((1,1-difluorobutan-2-yl)amino)ethyl)-1H-pyrazole-3-carboxylic acid (30 mg, crude product), the crude product was directly used in the next reaction. ES-API: [M+H]⁺=408.1.
Step 8: 5-chloro-1-(2,6-difluorobenzyl)-4-(2-((1,1-difluorobutan-2-yl)amino)ethyl)-1H-pyrazole-3-carboxylic acid (30 mg, crude product) was dissolved in dichloromethane (2 mL), N,N-diisopropylethylamine (0.5 mL) and HATU (33 mg, 0.08 mmol) were added. Under stirring, the reaction solution was proceeded at room temperature for 1 hour. The reaction solution was extracted with dichloromethane, concentrated under reduced pressure, and the crude product was purified by thin layer chromatography (petroleum ether/ethyl acetate=3/1) to obtain 3-chloro-2-(2,6-difluorobenzyl)-6-(1,1-difluorobutan-2-yl)-2,4,5,6-tetrahydro-7H-pyrazolo[3,4-c]pyridin-7-one (Z15, 12.1 mg, yield: 31%). ¹H NMR (400 MHz, CDCl³) δ 7.40-7.14 (m, 1H), 6.90 (t, J=8.0 Hz, 2H), 6.10-5.65 (m, 1H), 5.45 (s, 2H), 4.77 (d, J=14.6 Hz, 1H), 3.65-3.36 (m, 2H), 2.74 (td, J=6.4, 3.3 Hz, 2H), 1.87-1.71 (m, 2H), 0.95 (t, J=7.5 Hz, 3H). ES-API: [M+H]⁺=390.1.

### Example 16: Preparation of 6-(2-(2-benzyl-3-chloro-7-oxo-2,4,5,7-tetrahydro-6H-pyrazolo[3,4-c]pyridine-6-yl)propoxy)nicotinonitrile (Z16)

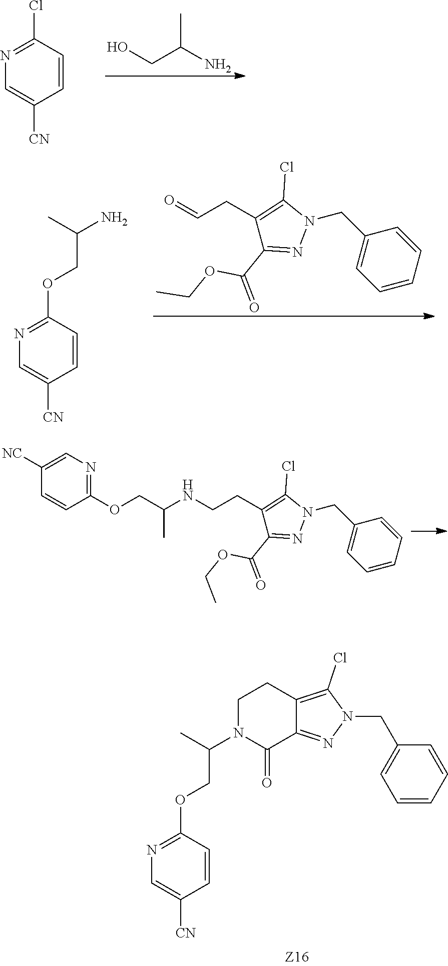 [0311] Step 1: Sodium hydrogen (560 mg, 13.98 mmol) and 6-chloronicotinonitrile (1384 mg, 9.99 mmol) were added successively into a solution of 2-aminopropan-1-ol (350 mg, 4.66 mmol) in N,N-dimethylformamide (20 mL), the reaction mixture was heated to 70° C. and stirred overnight, When LCMS detected the reaction was completed, the reaction solution was cooled to room temperature, water (30 mL) was added, the reaction solution was extracted with ethyl acetate (20 mL×3). The combined organic layer was washed with water (20 mL×2), dried over anhydrous sodium sulfate, filtered and concentrated under reduced pressure to obtain 6-(2-aminopropoxy)nicotinonitrile (319 mg, yield: 39%). ES-API: [M+H]⁺=178.1.
Step 2: Under nitrogen protection, 2-methylpyridineborane (66 mg, 0.612 mmol) was added to a solution of ethyl 1-benzyl-5-chloro-4-(2-oxyethyl)-1H-pyrazole-3-carboxylate (200 mg, 0.652 mmol), (2-aminopropoxy)nicotinonitrile (127 mg, 0.72 mmol), acetic acid (0.9 mL, 0.468 mmol) in methanol (9 mL, 0.468 mmol). The reaction mixture was stirred at room temperature for 3 hours. LCMS detected that the reaction was complete. The reaction solution was quenched by adding saturated sodium bicarbonate (30 mL) and extracted with ethyl acetate (30 mL×3). The organic phase was dried with anhydrous sodium sulfate, filtered, and concentrated under reduced pressure to obtain ethyl 1-benzyl-5-chloro-4-(2-((1-((5-cyanopyridin-2-yl)oxy)propan-2-yl)amino)ethyl)-1H-pyrazole-3-carboxylate (147 mg, crude product), ES-API: [M+H]⁺=468.2.
Step 3: Under nitrogen protection, trimethylaluminum solution in toluene(0.91 mL, 0.910 mmol, 1M in Tol) was added to a solution of ethyl 1-benzyl-5-chloro-4-(2-((1-((5-cyanopyridin-2-yl)oxy)propan-2-yl)amino)ethyl)-1H-pyrazole-3-carboxylate (147 mg, 0.314 mmol) in toluene (8 mL). The reaction mixture was heated to 100° C. and stirred for 4 hours. LCMS detected that the reaction was complete. The reaction solution was cooled to room temperature. Water (30 mL), ethyl acetate (30 mL), and saturated potassium sodium tartrate solution (30 mL) were added to the reaction solution in sequence. The organic phase was separated, dried over anhydrous sodium sulfate, filtered, and concentrated under reduced pressure. The crude product was purified by column chromatography (ethyl acetate: petroleum ether=0˜65%) to obtain a light yellow solid 6-(2-(2-benzyl-3-chloro-7-oxo-2,4,5,7-tetrahydro-6H-pyrazolo[3,4-c]pyridin-6-yl)propoxy)nicotinonitrile (Z16, 18.54 mg, two-step yield 7%). ES-API: [M+H]⁺=422.1. ¹H NMR (400 MHz, DMSO-d₆) δ 8.69 (d, J=1.6 Hz, 1 H), 8.14-8.11 (m, 1 H), 7.38-7.36 (m, 2 H), 7.33-7.30 (m, 1 H), 7.22 (d, J=5.6 Hz, 2 H), 6.96 (d, J=6.4 Hz, 1 H), 5.42 (s, 2 H), 5.06-5.02 (m, 1 H), 4.46-4.45 (m, 2 H), 3.56-3.52 (m, 2 H), 2.69-2.62 (m, 2 H), 1.21(d, J=5.6 Hz, 3 H).

### Example 17: Preparation of 1-(2,6-difluorobenzyl)-6-(1,1-difluoropropan-2-yl)-3-(trifluoromethyl)-1,4,5,6-tetrahydro-7H-pyrazolo[3,4-c]pyridin-7-one (Z17)

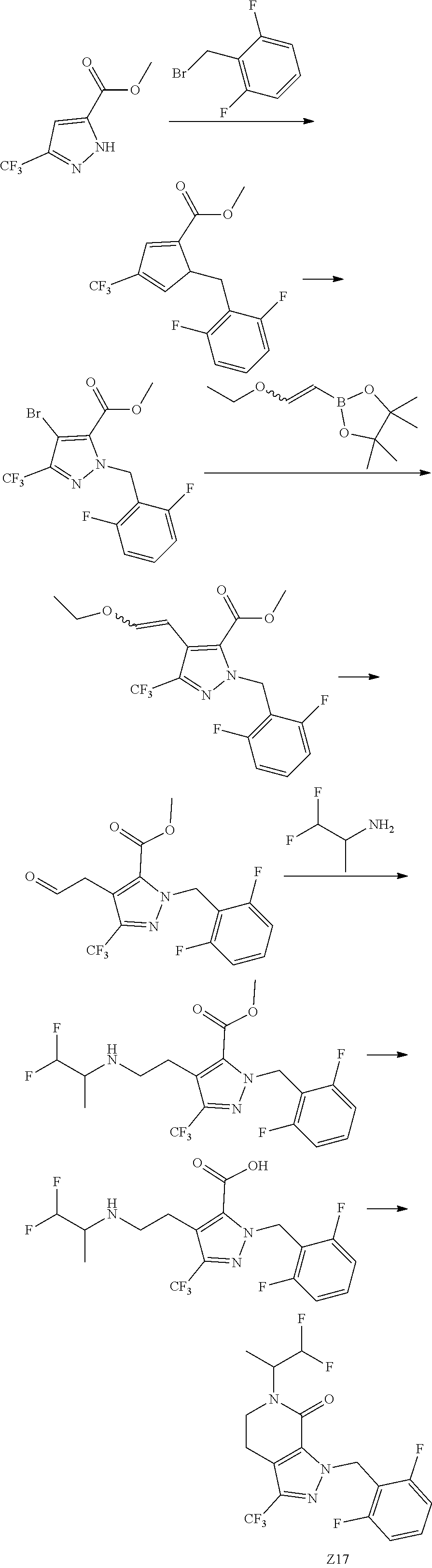 [0315] Step 1: 3-(trifluoromethyl)-1H-pyrazole-5-carboxylic acid methyl ester (500 mg, 2.58 mmol, 1 eq), 2-(bromomethyl)-1,3-difluorobenzene (534 mg, 2.58 mmol, leq) and potassium carbonate (712 mg, 5.16 mmol, 2.0 eq) were dissolved in 10 mL DMF and the reaction was proceeded at room temperature for 4 hours.

Ethyl acetate was added, the reaction solution was washed with water three times. The organic phase was dried over sodium sulfate, spin-dried under reduced pressure, and the crude product was separated by column chromatography (PE/EA=1/10) to obtain colorless oil 1-(2.6-difluorobenzyl)-3-(trifluoromethyl)-1H-pyrazole-5-carboxylic acid methyl ester (120 mg, yield: 12.1%). ES-API: [M+H]⁺=321.1.
Step 2: 1-(2,6-difluorobenzyl)-3-(trifluoromethyl)-1H-pyrazole-5-carboxylic acid methyl ester (120 mg, 0.375 mmol, 1.0 eq) and NBS (100 mg, 0.563 mmol, 1.5 eq) were dissolved in acetic acid (3 mL) and nitric acid (0.1 mL), 120° C., microwave reacted for 30 minutes. Ethyl acetate (60 mL) was added, the reaction solution was washed with saturated sodium bicarbonate solution (10 mL×2), and washed twice with water (10 mL×2). The organic phases were combined, dried over anhydrous sodium sulfate, and concentrated sequentially to obtain brown oily substance 4-bromo-1-(2,6-difluorobenzyl)-3-(trifluoromethyl)-1H-pyrazole-5-carboxylic acid methyl ester (160 mg, crude product), which was used directly in next reaction. ES-API: [M+H]⁺=399.
Step 3: Under nitrogen protection, 4-bromo-1-(2,6-difluorobenzyl)-3-(trifluoromethyl)-1H-pyrazole-5-carboxylic acid methyl ester (160 mg, 0.375 mmol), 2-(2-ethoxyvinyl)-4,4,5,5-tetramethyl-1,3,2-dioxaborane (149 mg, 0.75 mmol), Pd(dppf)C12(27 mg, 0.0375 mmol) and potassium carbonate (155 mg, 1.125 mmol) were dissolved in dioxane (10 mL) and water (1 mL), heated to 100° C., and the reaction was proceeded for 16 hours. Ethyl acetate (150 mL) was added, the reaction solution was washed twice with water (15 ml×2). The organic phases were combined, dried over anhydrous sodium sulfate, evaporated to dryness, and the crude product was purified by column chromatography (EA/PE=0˜10%) to obtain colorless oily substance 1-(2,6-difluorobenzyl)-4-(2-ethoxyvinyl)-3-(trifluoromethyl)-1H-pyrazole-5-carboxylic acid methyl ester (80 mg yield: 50%.). ES-API: [M+H]⁺=391.1.
Step 4: 1-(2,6-difluorobenzyl)-4-(2-ethoxyvinyl)-3-(trifluoromethyl)-1H-pyrazole-5-carboxylic acid methyl ester (80 mg, 0.21 mmol, 1.0 eq) was dissolved in THF (4 mL) and 6N HCl (1 mL) was slowly added dropwise. The solution was colorless and transparent, The reaction solution was stirred at 50° C. for 2 hours. 10 ml of purified water was added to the reaction bottle, 30 ml of ethyl acetate was added and extracted three times. The organic phases were combined, dried over anhydrous sodium sulfate, and spin-dried to obtain 1-(2,6-difluorobenzyl)-4-(2-oxyethyl)-3-(trifluoromethyl)-1H-pyrazole-5-carboxylic acid methyl ester (80 mg, crude product), which was used directly in the next reaction. ES-API: [M+H]⁺=363.1.
Step 5: 1-(2,6-difluorobenzyl)-4-(2-oxyethyl)-3-(trifluoromethyl)-1H-pyrazole-5-carboxylic acid methyl ester (80 mg, 0.21 mmol) and 1,1-difluoropropan-2-amine (30 mg, 0.32 mmol) were dissolved in methanol (2 ml), boranemethylpyridine complex (27 mg, 0.25 mmol) was added. Under stirring, the reaction was proceeded at room temperature for 2 hours. 10 ml of water was added to the reaction solution, 50 ml of ethyl acetate was added and extracted three times. The organic phase was washed with 10 ml of saturated brine, dried over anhydrous sodium sulfate, evaporated to dryness, and separated by column chromatography (EA/PE=0%-35%) to obtain a colorless oil of 1-(2,6-difluorobenzyl)-4-(2-((1,1-difluoropropan-2-yl)amino)ethyl)-3-(trifluoromethyl)-1H-pyrazole-5-carboxylic acid methyl ester (40 mg, yield: 41.3%). ES-API: [M+H]⁺=442.12.
Step 6: 1-(2,6-difluorobenzyl)-4-(2-((1,1-difluoropropan-2-yl)amino)ethyl)-3-(trifluoromethyl)-1H-pyrazole-5-carboxylic acid methyl ester (40 mg, 0.09 mmol) was dissolved in 10 ml tetrahydrofuran and 1 ml methanol, stirred evenly. The solution was colorless and transparent, 1N LiOH solution (2.5 mL) was added to the reaction solution, the reaction was proceeded at room temperature for 2 hours under stirring. 1N HCl was used to adjust the reaction solvent to weak acidity. The solution changes from colorless and clear to white turbidity and spin-dried to obtain 1-(2,6-difluorobenzyl)-4-(2-((1,1-difluoropropan-2-yl)amino)ethyl)-3-(trifluoromethyl)-1H-pyrazole-5-carboxylic acid (80 mg, crude). ES-API: [M+H]⁺=428.1.
Step 7: 1-(2,6-difluorobenzyl)-4-(2-((1,1-difluoropropan-2-yl)amino)ethyl)-3-(trifluoromethyl)-1H-pyrazole-5-carboxylic acid (80 mg, 0.09 mmol) was dissolved in DMF (2 mL), TEA (36 mg, 0.36 mmol) and HATU (42 mg, 0.11 mmol) were added, and the reaction was proceeded at room temperature for 16 hours.

The reaction was poured into 50 mL of water, extracted three times with 50 mL of ethyl acetate, washed once with 50 mL of saturated brine. The organic phase was dried over anhydrous sodium sulfate, concentrated, and the crude product was subjected to thin layer chromatography to obtain 1-(2,6-difluorobenzyl)-6-(1,1-difluoropropan-2-yl)-3-(trifluoromethyl)-1,4,5,6-tetrahydro-7H-pyrazolo[3,4-c]pyridin-7-one (Z17, 14 mg, yield: 38.1%). ¹H NMR (400 MHz, CDCl₃) δ 7.32 (ddd, J=8.5, 6.3, 2.1 Hz, 1H), 6.91 (t, J=8.0 Hz, 2H), 6.07-5.71 (m, 1H), 5.53 (s, 2H), 4.96 (dddd, J=19.9, 10.0, 7.2, 2.8 Hz, 1H), 3.60 (t, J=6.5 Hz, 2H), 2.94 (q, J=6.7 Hz, 2H), 1.34 (d, J=7.2 Hz, 3H). ES-API: [M+H]⁺=410.1.

### Example 18: Preparation of 3-chloro-2-(2,4-difluorobenzyl)-6-(1,1-difluoropropan-2-yl)-2,4,5,6-tetrahydro-7H-pyrazolo[3,4-c]pyridin-7-one (Z18)

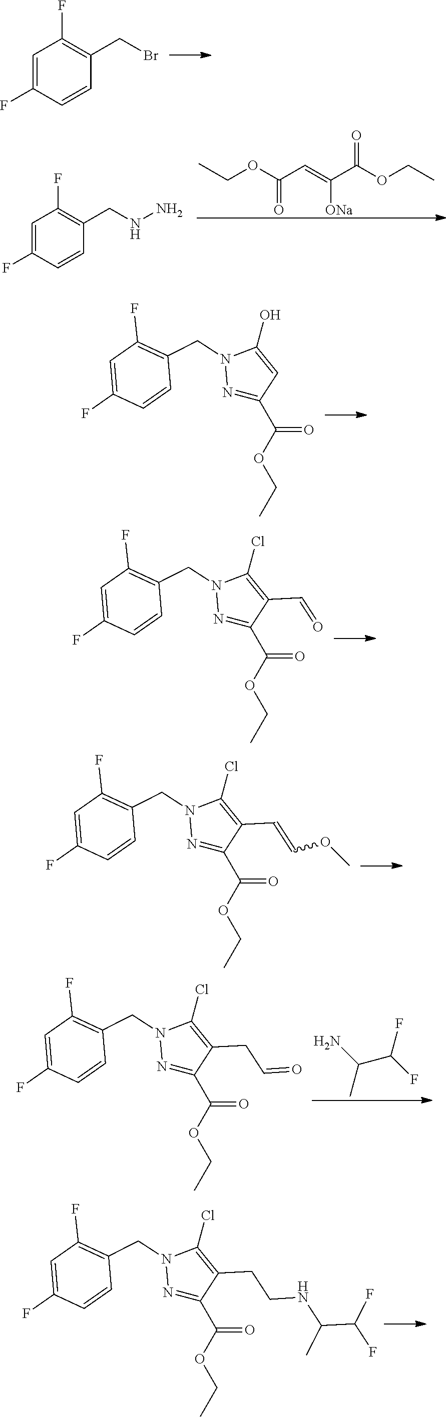 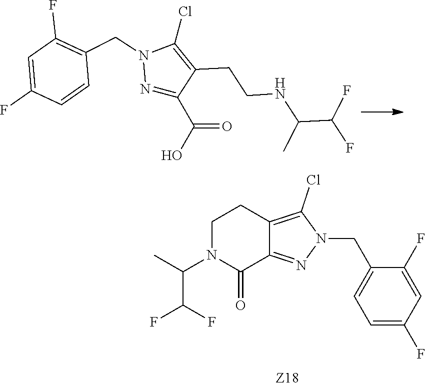 [0325] Step 1: 1-(bromomethyl)-2,4-difluorobenzene (20 g, 106 mmol) was dissolved in ethanol (150 mL) solution, and then added dropwise to hydrazine hydrate (105 g, 2116 mmol). Then the temperature was raised to 50° C. and the reaction was proceeded for 1 hour. The crude product was spin-dried and purified by thin layer chromatography (DCM/MeOH=20/1, Rf=0.6) to obtain the product (2,4-difluorobenzyl)hydrazine (12.3 g, yield: 78%), ES-API: [M+H]⁺=159.1.
Step 2: (2,4-difluorobenzyl)hydrazine (12.3 g, 77.8 mmol) was dissolved in acetic acid (50 mL), then diethyl oxaloacetate sodium salt (12.25 g, 58.3 mmol) was added dropwise, with the temperature raised to 100° C., the reaction solution was stirred for 16 hours. After the reaction was completed, the acetic acid was spin-dried, dichloromethane was added for extraction, and conducted with thin layer chromatography (PE/EA=5/1, Rf=0.5) to obtain product ethyl 1-(2,4-difluorobenzyl)-5-hydroxy-1H-pyrazole-3-carboxylate (10 g, yield: 63%), ES-API: [M+H]⁺=283.1.
Step 3: ethyl 1-(2,4-difluorobenzyl)-5-hydroxy-1H-pyrazole-3-carboxylate (10 g, 35.4 mmol) was dissolved in phosphorus oxychloride (50 mL) at 0° C. Then N,N-dimethylformamide (10 mL) was added, the temperature was raised to 90° C. and the reaction solution was stirred for 16 hours. After the reaction was completed, the phosphorus oxychloride was spin-dried, ice water was added, the reaction solution was extracted with dichloromethane, and thin layer chromatography was performed (PE/EA=5/1, Rf=0.5) to obtain product ethyl 5-chloro-1-(2,4-difluorobenzyl)-4-formyl-1H-pyrazole-3-carboxylate (4.3 g, yield: 37%), ES-API: [M+H]⁺=329.7.
Step 4: potassium tert-butoxide (5.1 g, 45.75 mmol) was dissolved in tetrahydrofuran solution (150 mL), then (methoxymethyl)triphenylphosphorus chloride (10.95 g, 32.0 mmol) was added dropwise under argon protection, then the reaction solution was cooled to −50° C., the reaction was proceeded for 30 min under stirring. ethyl 5-chloro-1-(2,4-difluorobenzyl)-4-formyl-1H-pyrazole-3-carboxylate (2.5 g, 7.6 mmol), which was dissolved in tetrahydrofuran solution was added dropwise at −50° C. The reaction solution was slowly warmed up and the reaction was proceeded for 1 hour. After the reaction was completed, the reaction solution was extracted with dichloromethane and thin layer chromatography was performed (PE/EA=5/1, Rf=0.5) to obtain the product ethyl 5-chloro-1-(2,4-difluorobenzyl)-4-(2-methoxyvinyl)-1H-pyrazole-3-carboxylate (250 mg, yield: 9.21%), ES-API: [M+H]⁺=357.1.
Step 5: ethyl 5-chloro-1-(2,4-difluorobenzyl)-4-(2-methoxyvinyl)-1H-pyrazole-3-carboxylate (135 mg, 0.38 mmol) was dissolved in tetrahydrofuran (2 mL), then 6N HCl (2 mL) was added dropwise, the temperature was raised to 50° C. and the reaction was proceeded for two hours. After the reaction was completed, quenched with water, extracted with ethyl acetate and spin-dried to obtain the crude product ethyl 5-chloro-1-(2,4-difluorobenzyl)-4-(2-oxyethyl)-1H-pyrazole-3-carboxylate (129 mg, Yield: 100%), ES-API: [M+H]⁺=343.1.
Step 6: ethyl 5-chloro-1-(2,4-difluorobenzyl)-4-(2-oxyethyl)-1H-pyrazole-3-carboxylate (129 mg, 0.38 mmol) was dissolved in methanol (0.5 mL), then 1,1-difluoropropan-2-amine (0.5 mL) and 2-methylpyridineborane complex (25 mg, 0.23 mmol) was added, and the reaction was proceeded at room temperature for 1 hour. The reaction solution was spin-dried to obtain the crude product ethyl 5-chloro-1-(2,4-difluorobenzyl)-4-(2-((1,1-difluoropropan-2-yl)amino)ethyl)-1H-pyrazole-3-carboxylate (135 mg, yield: 82%), ES-API: [M+H]⁺=422.1.
Step 7: ethyl 5-chloro-1-(2,4-difluorobenzyl)-4-(2-((1,1-difluoropropan-2-yl)amino)ethyl)-1H-pyrazole-3-carboxylate (135 mg, 0.32 mmol) was dissolved in tetrahydrofuran (2 mL), then lithium hydroxide hydrate (50 mg, 1.19 mmol) and water (2 mL) were added, and the reaction was proceeded at room temperature for 2 hours. After the reaction was completed, dilute hydrochloric acid was added to adjust the pH to weak acidity, and then the reaction solution was spin-dried to obtain the crude product 5-chloro-1-(2,4-difluorobenzyl)-4-(2-((1,1-difluoropropan-2-yl)amino)ethyl)-1H-pyrazole-3-carboxy acid (125 mg, Yield: 100%), ES-API: [M+H]⁺=394.1.
Step 8: 5-chloro-1-(2,4-difluorobenzyl)-4-(2-((1,1-difluoropropan-2-yl)amino)ethyl)-1H-pyrazole-3-carboxylic acid (125 mg, 0.32 mmol) was dissolved in N,N-dimethylformamide (2 mL), and then N,N-diisopropylethylamine (0.5 mL) was added dropwise to adjust the pH to weak alkaline. Then HATU reagent (140 mg, 0.37 mmol) was added, the reaction was proceeded at room temperature for two hours, and then the crude product was purified by preparative high-performance liquid chromatography (column: Ultimate XB-C18, 50*250 mm, 10 um; A: purified water B: pure acetonitrile flow rate: 80 ml/min, wavelength: 210 nm) to obtain 3-chloro-2-(2,4-difluorobenzyl)-6-(1,1-difluoropropan-2-yl)-2,4,5,6-tetrahydro-7H-pyrazolo[3,4-c]pyridin-7-one(Z18, 20.0 mg, yield: 15.53%). ¹H NMR (400 MHz, CDCl₃) δ 7.18 (q, J=7.7 Hz, 1H), 6.81 (t, J=8.7 Hz, 2H), 6.04-5.73 (m, 1H), 5.41 (s, 2H), 5.11-4.93 (m, 1H), 3.58 (t, J=6.5 Hz, 2H), 2.73 (q, J=6.3 Hz, 2H), 1.34 (d, J=7.3 Hz, 3H). ES-API: [M+H]⁺=376.1.

### Example 19: Preparation of 3-chloro-6-(1,1-difluoropropan-2-yl)-2-(2-fluorobenzyl)-2,4,5,6-tetrahydro-7H-pyrazolo[3,4-c]pyridin-7-one (Z19)

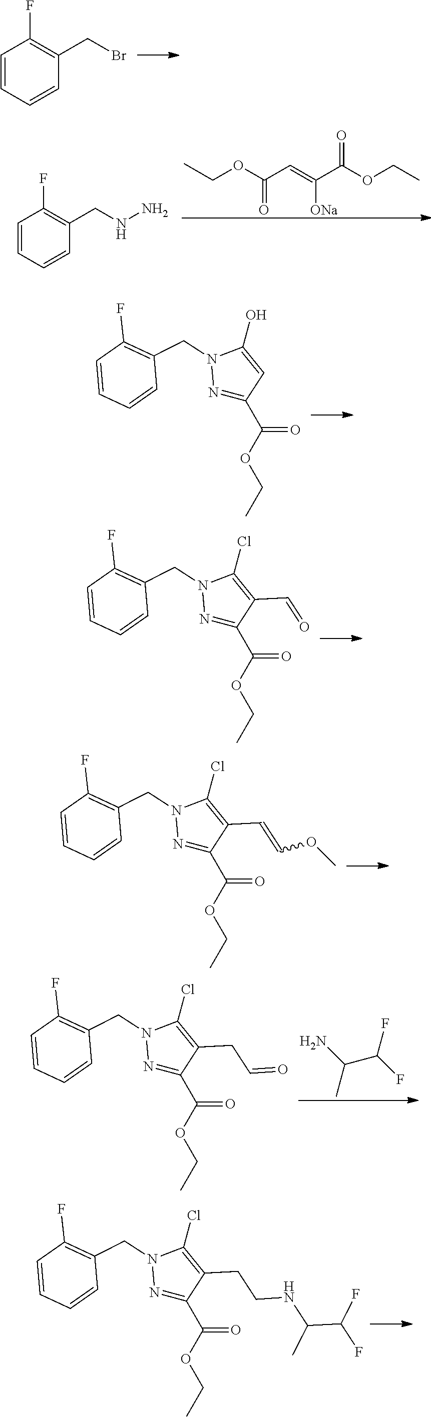 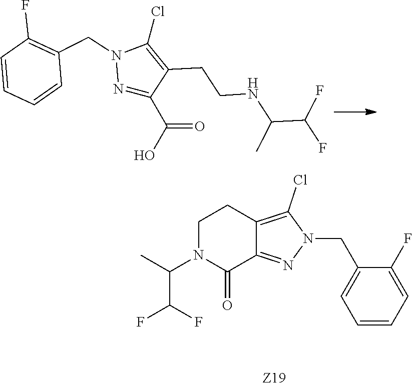 [0334] Step 1: 1-(bromomethyl)-2-fluorobenzene (20 g, 106 mmol) was dissolved in ethanol solution (150 mL), and then added dropwise to hydrazine hydrate (105 g, 2116 mmol). Then the temperature was raised to 50° C. and the reaction was proceeded for 1 hour. The crude product was spin-dried and purified by thin-layer chromatography (DCM/MeOH=20/1, Rf=0.6) to obtain the product (2-fluorobenzyl)hydrazine (9 g, yield: 60.42%), ES-API: [M +H]⁺=141.1.
Step 2: (2-fluorobenzyl)hydrazine (9.0 g, 64.3 mmol) was dissolved in acetic acid (50 mL), then diethyl oxaloacetate sodium salt (10.0 g, 48.2 mmol) was added dropwise, the temperature was raised to 100° C. and the reaction solution was stirred for 16 hours. After the reaction was completed, the acetic acid was spin-dried, dichloromethane was added for extraction. The reaction solution was subjected to thin layer chromatography (PE/EA=5/1, Rf=0.5) to obtain the product ethyl 1-(2-fluorobenzyl)-5-hydroxy-1H-pyrazole-3-carboxylate (6.7 g, yield: 39.48%), ES-API: [M+H]⁺=265.1.
Step 3: ethyl 1-(2-fluorobenzyl)-5-hydroxy-1H-pyrazole-3-carboxylate (6.7 g, 25.4 mmol) was dissolved in phosphorus oxychloride (50 mL) at 0° C., then N,N-dimethylformamide (5 mL) was added. The temperature was raised to 90° C. and the reaction solution was stirred for 16 hours. After the reaction was completed, phosphorus oxychloride was spin-dried, ice water was added. The reaction solution was extracted with dichloromethane, and thin layer chromatography was performed (PE/EA=5/1, Rf=0.4) to obtain the product ethyl 5-chloro-1-(2-fluorobenzyl)-4-formyl-1H-pyrazole-3-carboxylate (6.6 g, yield: 83.89%), ES-API: [M+H]⁺=310.1.
Step 4: potassium tert-butoxide (5.4 g, 48.4 mmol) was dissolved in tetrahydrofuran solution (140 mL), and then added into (methoxymethyl)triphenylphosphorus chloride (12.45 g, 36.3 mmol) dropwise under argon protection, after cooled to −50° C., the reaction was proceeded under stirring for 30 minutes. Ethyl 5-chloro-1-(2-fluorobenzyl)-4-formyl-1H-pyrazole-3-carboxylate (2.5 g, 8.05 mmol), which was dissolved in tetrahydrofuran solution was added dropwise at −50° C. The solution was rewarmed slowly and the reaction was proceeded for 1 hour. After the reaction was completed, the reaction solution was extracted with dichloromethane and thin layer chromatography was performed (PE/EA=5/1, Rf=0.6) to obtain the product ethyl 5-chloro-1-(2-fluorobenzyl)-4-(2-methoxyvinyl)-1H-pyrazole-3-carboxylate (100 mg, yield: 3.67%), ES-API: [M+H]⁺=339.1.
Step 5: ethyl 5-chloro-1-(2-fluorobenzyl)-4-(2-methoxyvinyl)-1H-pyrazole-3-carboxylate (100 mg, 0.3 mmol) was dissolved in tetrahydrofuran (2 mL), then 6N HCl (2 mL) was added dropwise, the temperature was raised to 50° C., and the reaction was proceeded for 2 hours. After the reaction was completed, the reaction solution was quenched with water, extracted with ethyl acetate and spin-dried to obtain the crude product ethyl 5-chloro-1-(2-fluorobenzyl)-4-(2-oxyethyl)-1H-pyrazole-3-carboxylate (80 mg, yield: 83.46%), ES-API: [M+H]⁺=325.1.
Step 6: ethyl 5-chloro-1-(2-fluorobenzyl)-4-(2-oxyethyl)-1H-pyrazole-3-carboxylate (80 mg, 0.25 mmol) was dissolved in methanol (0.5 mL), then 1,1-difluoropropan-2-amine (0.5 mL) and 2-methylpyridineborane complex (25 mg, 0.23 mmol) were added, the reaction was proceeded at room temperature for 1 hour, and spin-dried to obtain the crude product ethyl 5-chloro-4-(2-((1,1-difluoropropan-2-yl)amino)ethyl)-1-(2-fluorobenzyl)-1H-pyrazole-3-carboxylate (100 mg, yield: 100.00%), ES-API: [M+H]⁺=404.1.
Step 7: ethyl 5-chloro-4-(2-((1,1-difluoropropan-2-yl)amino)ethyl)-1-(2-fluorobenzyl)-1H-pyrazole-3-carboxylate (100 mg, 0.25 mmol) was dissolved in tetrahydrofuran (2 mL), then hydrated lithium hydroxide (50 mg, 1.19 mmol) and water (2 mL) were added, and the reaction was proceeded at room temperature for 2 hours. After the reaction was completed, dilute hydrochloric acid was added to adjust the pH to weak acidity, and then the reaction solution was spin-dried to obtain the crude product 5-chloro-4-(2-((1, 1-difluoropropan-2-yl)amino)ethyl)-1-(2-fluorobenzyl)-1H-pyrazole-3-carboxylic acid (95 mg, yield: 100.00%), ES-API: [M+H]⁺=376.1.
Step 8: 5-chloro-4-(2-(((1,1-difluoropropan-2-yl)amino)ethyl)-1-(2-fluorobenzyl)-1H-pyrazole-3-carboxylic acid (95 mg, 0.25 mmol) was dissolved in dichloromethane (2 mL), then N,N-diisopropylethylamine (0.5 mL) was added dropwise to adjust the pH to weak alkaline, and then HATU reagent (100 mg, 0.26 mmol) was added, the reaction was proceeded at room temperature for 2 hours, and then the crude product was prepared by preparative high-performance liquid chromatography (column: Ultimate XB-C18, 50*250 mm, 10 μm; A: purified water B: pure acetonitrile flow rate: 80 ml/min, wavelength: 210 nm) to obtain 3-chloro-6-(1,1-difluoropropan-2-yl)-2-(2-fluorobenzyl)-2,4,5,6-tetrahydro-7H-pyrazolo[3,4-c]pyridin-7-one (Z19, 6.1 mg, Y: 6.74%). ¹H NMR (400 MHz, CDCl₃) δ 7.26 (d, J=5.9 Hz, 1H), 7.13 (t, J=6.9 Hz, 1H), 7.10-7.02 (m, 2H), 5.89 (t, J=2.6 Hz, 1H), 5.48 (s, 2H), 5.11-4.97 (m, 1H), 3.59 (t, J=6.6 Hz, 2H), 2.74 (q, J=6.4 Hz, 2H), 1.35 (d, J=7.2 Hz, 3H). ES-API: [M+H]⁺⁼358.1.

### Example 20: Preparation of 3-chloro-2-(2,6-difluorobenzyl)-6-propionyl-2,4,5,6-tetrahydro-7H-pyrazolo[3,4-c]pyridin-7-one (Z20)

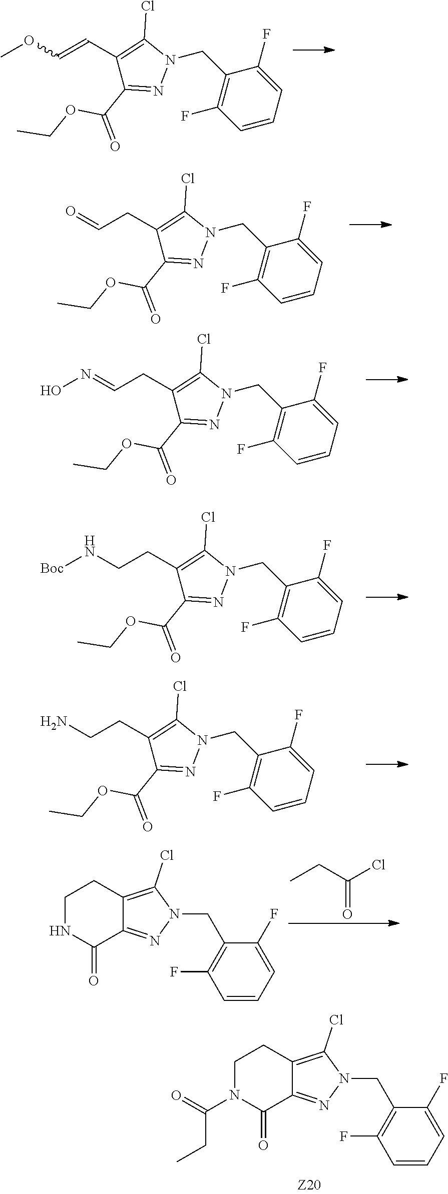 [0343] Step 1: ethyl 5-chloro-1-(2,6-difluorobenzyl)-4-(2-methoxyvinyl)-1H-pyrazole-3-carboxylate (1.3 g, 0.84 mmol) was dissolved in tetrahydrofuran (5 mL), and hydrochloric acid (5N, 4 mL) was added. Under stirring, the reaction was proceeded at 50° C. for 1 hour. The reaction solution was cooled to room temperature, extracted with ethyl acetate, and concentrated under reduced pressure to obtain crude product ethyl 5-chloro-1-(2,6-difluorobenzyl)-4-(2-oxyethyl)-1H-pyrazole-3-carboxylate (1.43 g, crude product). ES-API: [M+1]⁺=343.0.
Step 2: crude product ethyl 5-chloro-1-(2,6-difluorobenzyl)-4-(2-oxyethyl)-1H-pyrazole-3-carboxylate (1.43 g, 4.18 mmol) was dissolved in ethanol/water (5 mL/1.2 mL), sodium acetate (1.03 g, 12.54 mmol) and hydroxylamine hydrochloride (577 mg, 8.36 mmol) were added. Under stirring, the reaction was proceeded at 70° C. for 1 hour. The reaction solution was cooled to room temperature, concentrated under reduced pressure to remove ethanol, extracted with dichloromethane, and the organic phase was concentrated under reduced pressure to obtain ethyl (E)-5-chloro-1-(2,6-difluorobenzyl)-4-(2-(hydroxyimino)ethyl)-1H-pyrazole-3-carboxylate crude product (1.36 g, yield: 91%, yellow solid). ES-API: [M+1]⁺=343.0.
Step 3: ethyl (E)-5-chloro-1-(2,6-difluorobenzyl)-4-(2-(hydroxyimino)ethyl)-1H-pyrazole-3-carboxylate crude product (1.36 g, 3.82 mmol) was suspended in methanol (12 mL), and nickel chloride hexahydrate (1.09 g, 4.58 mmol) was added. Under stirring, the reaction was proceeded at room temperature for 30 minutes. The reaction solution was cooled to 0° C., and sodium borohydride (726 mg, 19.1 mmol) was added in batches. After the addition was completed, the reaction solution was raised to room temperature stirred and reacted for 1 hour. Then di-tert-butyl dicarbonate (1 g, 4.58 mmol) was added, and the reaction was proceeded at room temperature under stirring for 16 hours. The reaction solution was filtered through diatomaceous earth, and the filtrate was concentrated under reduced pressure. The obtained crude product was extracted with dichloromethane, and the organic phase was concentrated under reduced pressure to obtain a gray-green crude product which was purified by column chromatography (petroleum ether/ethyl acetate=5/1) to obtain ethyl 4-(2-((tert-butoxycarbonyl)amino)ethyl)-5-chloro-1-(2,6-difluorobenzyl)-1H-pyrazole-3-carboxylate (900 mg, Yield: 53%, white solid) MS-API: [M−100+1]⁺=344.0.
Step 4: ethyl 4-(2-((tert-butoxycarbonyl)amino)ethyl)-5-chloro-1-(2,6-difluorobenzyl)-1H-pyrazole-3-carboxylate (1.1 g, 2.48 mmol) was dissolved in dichloromethane (3 mL), and trifluoroacetic acid (2 mL) was added. The reaction was proceeded at room temperature under stirring for 1 hour. The reaction solution was directly concentrated under reduced pressure to obtain crude ethyl 4-(2-aminoethyl)-5-chloro-1-(2,6-difluorobenzyl)-1H-pyrazole-3-carboxylate (1.3 g , yield: 100%, light yellow liquid). ES-API: [M+1]⁺=344.1.
Step 5: ethyl 4-(2-aminoethyl)-5-chloro-1-(2,6-difluorobenzyl)-1H-pyrazole-3-carboxylate (1.3 g, 3.79 mmol) was dissolved in tetrahydrofuran/methanol/water (4 mL/2 mL/1 mL), lithium hydroxide monohydrate (477 mg, 11.37 mmol) was added. The reaction was proceeded at room temperature under stirring for 3 hours. The reaction solution was extracted with ethyl acetate and concentrated under reduced pressure to obtain 3-chloro-2-(2,6-difluorobenzyl)-2,4,5,6-tetrahydro-7H-pyrazolo[3,4-c]pyridin-7-one (630 mg, yield: 85%, white solid). ES-API: [M+1]⁺=298.0.
Step 6: 3-chloro-2-(2,6-difluorobenzyl)-2,4,5,6-tetrahydro-7H-pyrazolo[3,4-c]pyridin-7-one (120 mg , 0.40 mmol) was dissolved in dichloromethane (3 mL), and triethylamine (1 mL), DMAP (catalytic amount) and propionyl chloride (368 mg, 4.0 mmol) were added. The reaction was proceeded at 40° C. under stirring for 3 hours. The reaction solution was cooled to room temperature, quenched with ice water, extracted with dichloromethane, and the organic phase was concentrated under reduced pressure. The crude product was purified by thin layer chromatography (petroleum ether/ethyl acetate=4/1) to obtain 3-chloro-2-(2,6-difluorobenzyl)-6-propionyl-2,4,5,6-tetrahydro-7H-pyrazolo[3,4-c]pyridin-7-one (Z20, 36.3 mg, yield: 18%, light yellow solid). ¹H NMR (400 MHz, CDCl₃) δ 7.30 (p. J=7.5 Hz, 1H), 6.90 (t, J=8.0 Hz, 2H), 5.47 (s, 2H), 4.16 (t, J=6.3 Hz, 2H), 2.98 (q, J=7.3 Hz, 2H), 2.71 (t, J=6.3 Hz, 2H), 1.16 (t, J=7.3 Hz, 3H). ES-API: [M+1]⁺=354.0.

### Example 21: Preparation of 3-chloro-6-(1,1-difluoropropan-2-yl)-2-(2,4,5-trifluorobenzyl)-2,4,5,6-tetrahydro-7H-pyrazolo[3,4-c]pyridin-7-one (Z21)

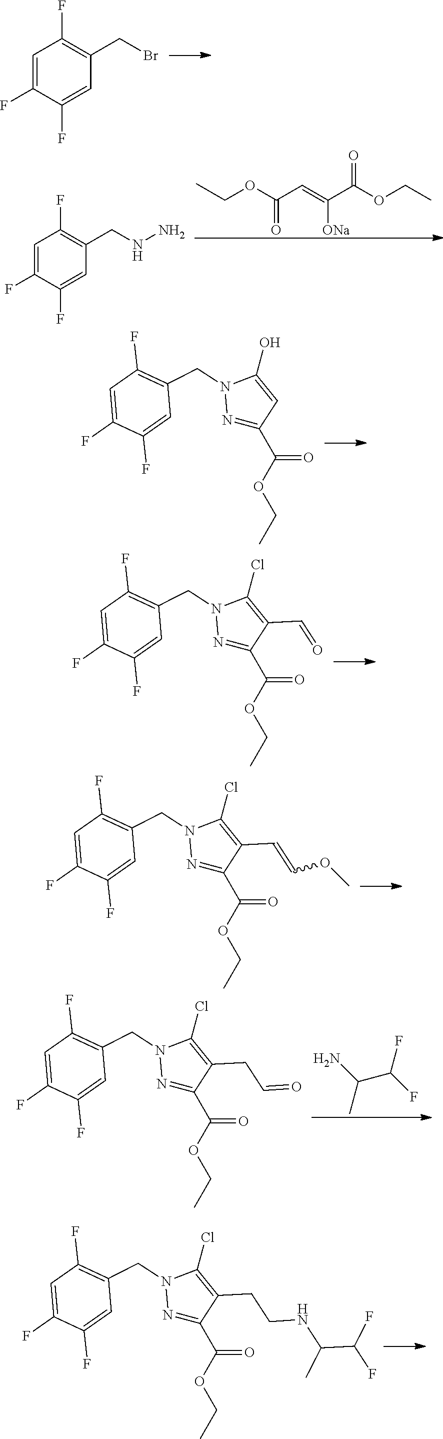 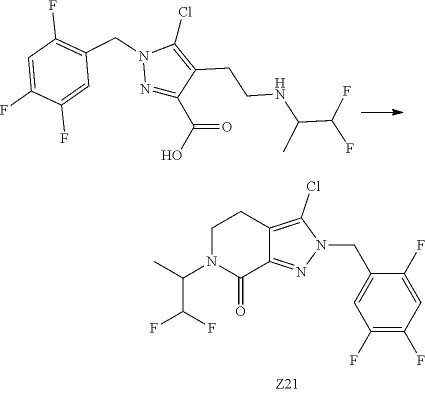 [0350] Step 1: hydrazine hydrate (101.6 g, 2.03 mol) was placed in a 2L three-necked flask and the reaction was proceeded at 50° C. under stirring for 30 minutes. 1-(bromomethyl)-2,4,5-trifluorobenzene (84 g, 0.37mol) was dissolved in 840 mL of absolute ethanol and the solution was slowly added dropwise into the reaction mixture. After the addition was completed, the reaction was proceeded at 50-60° C. for 1 hour. TLC (PE) confirmed that compound 1 disappeared completely. The reaction solvent was spin-dried under reduced pressure, with water (500 mL) added, extracted with dichloromethane (500 mL×3). The organic phase was washed with saturated brine (500 mL), dried over anhydrous sodium sulfate, and evaporated to dryness to obtain a yellow solution (60 g, yield: 50%), which was used directly in the next step. ES-API: [M+H]⁺=176.14.
Step 2: diethyl oxaloacetate sodium salt (39.9 g, 190 mmol) was dissolved in acetic acid (420 mL), stirred for 30 minutes, and the reaction solution became clear. (2,4,5-trifluorobenzyl)hydrazine (60 g, 380 mmol) was added to the reaction solution, heated to 100° C., and the reaction was proceeded for 2 hours. The reaction solvent was spin-dried under reduced pressure, dissolved in ethyl acetate (400 mL), washed with hydrochloric acid (2N, 300 mL), and the aqueous phase was extracted with ethyl acetate (400 mL×2). The organic phases were combined, washed with water (300 mL×2) and saturated brine (300 mL) in sequence, dried over anhydrous sodium sulfate, evaporated to 50 mL-80 mL, cooled to room temperature and stirred for 1 hour. The solution was filtered and the solid was washed with ethyl acetate (20 mL×3). After drained, the white solid product ethyl 5-hydroxy-1-(2,4,5-trifluorobenzyl)-1H-pyrazole-3-carboxylate (24.6 g, yield: 45%) was obtained, ES-API: [M+H]⁺=300.23.
Step 3: ethyl 5-hydroxy-1-(2,4,5-trifluorobenzyl)-1H-pyrazole-3-carboxylate (24.6 g, 87.23 mmol) was dissolved in N,N-dimethylformamide (57 mL), phosphorus oxychloride (175 mL) was added dropwise. After the addition was completed, the reaction solution was heated to 90° C. and the reaction was proceeded overnight, After confirming that the raw materials disappeared completely and the intermediate (MS: 338) was less than 1%, the reaction solution was evaporated to dryness under reduced pressure, slowly added to water and the temperature was controlled within 30° C. (ice was added). The pH was adjusted to 7-8 with solid sodium bicarbonate. The reaction solution was extracted with ethyl acetate (500 mL×4), the organic phase was washed with water (500 mL), saturated brine (500 mL), dried over anhydrous sodium sulfate, and evaporated to dryness. The product was off-white to yellow solid ethyl 5-chloro-4-formyl-1-(2,4,5-trifluorobenzyl)-1H-pyrazole-3-carboxylate (26.6 g, yield: 16%). ES-API: [M+H]⁺=346.69.
Step 4: (methoxymethyl)triphenylphosphonium chloride (96.0 g, 280 mmol) was suspended in tetrahydrofuran (300 mL), under protection of nitrogen, cooled to 0-10° C., potassium tert-butoxide (28.8 g, 25.6 mmol) was added, the temperature was controlled at 20° C., and the reaction solution was stirred for 20 minutes after the addition was completed (the reaction solution turned brown-red). With the reaction solution cooled to −50° C., ethyl 5-chloro-4-formyl-1-(2,4,5-trifluorobenzyl)-1H-pyrazole-3-carboxylate (20.0 g, 61.1 mmol) was dissolved in THF (100 mL), wherein the reaction solution was added dropwise, and the temperature was controlled within −30° C. After the addition, the temperature was naturally raised to room temperature and the reaction was proceeded for 2 hours (the reaction solution was yellow suspension). The reaction solution was cooled to 0-10° C., with saturated ammonium chloride (200 mL) added, stirred for 10 minutes, liquid separation extraction was performed. The aqueous phase was extracted with ethyl acetate (300 mL×2). The organic phases were combined, washed with saturated sodium chloride (300 mL), dried over anhydrous sodium sulfate, evaporated to dryness and purified by column chromatography (ethyl acetate/petroleum ether=5%-15%) to obtain the white solid product ethyl 5-chloro-4-(2-methoxyvinyl)-1-(2,4,5-trifluorobenzyl)-1H-pyrazole-3-carboxylate (7 g, yield: 55%). ES-API: [M+H]⁺=374.74.
Step 5: ethyl 5-chloro-4-(2-methoxyvinyl)-1-(2,4,5-trifluorobenzyl)-1H-pyrazole-3-carboxylate (7 g, 19.6 mmol) was dissolved in tetrahydrofuran (20 ml), and 6N hydrochloric acid (10 ml) was slowly added dropwise. The solution was colorless and transparent, The reaction was proceeded at 50° C. under stirring for 4 hours. After 4 hours, the solution was found to be light yellow, and LCMS monitored the reaction completed. 100 ml of pure water was added into the reaction bottle. The reaction solution changed from colorless and transparent to white turbid. The reaction solution was extracted with ethyl acetate (30 mL×3), washed with saturated brine (30 mL×1), dried with anhydrous sodium sulfate, and spin-dried to obtain the crude of product ethyl 5-chloro-4-(2-oxyethyl)-1-(2,4,5-trifluorobenzyl)-1H-pyrazole-3-carboxylate (7 g, 20.5 mmol) and 1,1-difluoropropan-2-amine (7 g, 100%), the crude was directly used in the next reaction. ES-API: [M+H]⁺=360.72.
Step 6: ethyl 5-chloro-4-(2-oxyethyl)-1-(2,4,5-trifluorobenzyl)-1H-pyrazole-3-carboxylate (7 g, 20.5 mmol) and 1,1-difluoropropan-2-amine (1.98 g, 24.4 mmol) were dissolved in methanol (20 ml) and stirred at 0° C. for 1 hour. After lhour, borane methylpyridine complex (3.24 g, 30.56 mmol) was added, the reaction was stirred at 0° C. for 1 hour and LCMS monitored the reaction completed. 100 ml of water was added to the reaction solution, 50 ml of ethyl acetate was added, and the reaction solution was extracted three times. The organic phase was washed with 50 ml of saturated brine, dried with anhydrous sodium sulfate, evaporated to dryness, and purified by column chromatography (ethyl acetate/petroleum ether=1/1) to obtain the product ethyl 5-chloro-4-(2-((1,1-difluoropropan-2-yl)amino) ethyl)-1-(2,4,5-trifluorobenzyl)-1H-pyrazole-3-carboxylate (2.4 g, yield: 13%) white crystals. ES-API: [M+H]⁺=439.81.
Step 7: ethyl 5-chloro-4-(2-((1,1-difluoropropan-2-yl)amino)ethyl)-1-(2,4,5-trifluorobenzyl)-1H-pyrazole-3-carboxylate (2.4 g, 5.46 mmol) was dissolved in 10 ml methanol and 3 ml water and stirred evenly. The solution was colorless and transparent, Lithium hydroxide monohydrate (460 mg, 10.94 mmol) was added. The reaction was proceeded at room temperature under stirring for 2 hours and LCMS monitored the reaction completed. 1N hydrochloric acid was used to adjust the reaction solvent to weak acidity. The solution changed from colorless and clear to white turbid, spin-dried to obtain product 5-chloro-4-(2-((1,1-difluoropropan-2-yl)amino)ethyl)-1-(2,4,5-trifluorobenzyl)-1H-pyrazole-3-carboxylic acid (2.4 g, crude product). ES-API: [M+H]⁺=411.75.
Step 8: 5-chloro-4-(2-((1,1-difluoropropan-2-yl)amino)ethyl)-1-(2,4,5-trifluorobenzyl)-1H-pyrazole-3-carboxylic acid (2.4 g, 6.1 mmol) was dissolved in dichloromethane (5 ml), 0.5 ml of N,N′-diisopropylethylamine, and 2-(7-aza-1H-benzotriazol-1-yl)-1,1,3,3-tetramethylurea hexafluorophosphate (4.8 g, 12.6 mmol) were added, the reaction solution changed from a light yellow clear state to some white solid precipitated, and the reaction was stirred at room temperature of 25° C. for 1 hour, and LCMS monitored the reaction completed. The reaction was poured into 50 ml of water, extracted three times with 50 ml of ethyl acetate, washed once with 50 ml of saturated brine, the organic phase was dried with anhydrous sodium sulfate, evaporated to dryness, and purified by column chromatography (ethyl acetate/petroleum ether=2/1) to obtain the product (750 mg, purity 85%) as a white solid. The white solid was slurried with 5 ml n-heptane and filtered. The solid was dissolved in 5 ml acetonitrile and freeze-dried to obtain the product 3-chloro-6-(1,1-difluoropropan-2-yl)-2-(2,4,5-trifluorobenzyl)-2,4,5,6-tetrahydro-7H-pyrazolo[3,4-c]pyridin-7-one (Z21, 60 mg, yield: 12%), purity 99.69%. ¹H NMR (400 MHz, CDCl₃) δ 7.36-7.25 (m, 1H), 6.90 (t, J=8.0 Hz, 1H), 5.88 (t, J=2.6 Hz, 1H), 5.46 (s, 2H), 5.06-4.92 (m, 1H), 3.57 (t, J=6.5 Hz, 2H), 2.73 (q, J=6.4 Hz, 2H), 1.33 (d, J=7.2 Hz, 3H). ES-API: [M+H]⁺=393.74.

### Example 22: Preparation of 3-chloro-6-(1,1-difluoropropan-2-yl)-2-(2,4,6-trifluorobenzyl)-2,4,5,6-tetrahydro-7H-pyrazolo[3,4-c]pyridin-7-one (Z22)

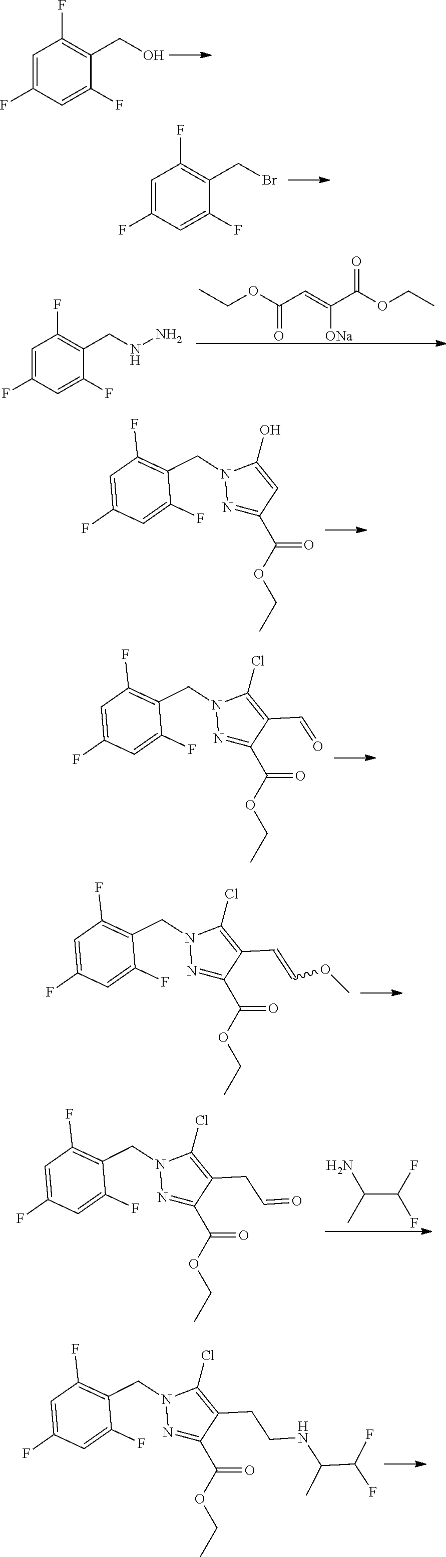 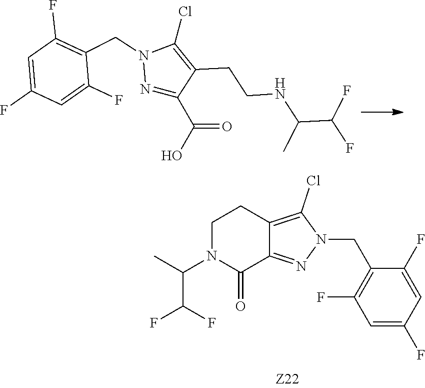 [0359] Step 1: triphenylphosphine (58.2 g, 222 mmol) was dissolved in dichloromethane (300 mL), cooled to 0° C., and NBS (40 g, 222 mmol) was added in batches. (2,4,6-Trifluorophenyl)methanol (30 g, 185 mmol) was dissolved in dichloromethane (100 mL) at room temperature and added dropwise to the reaction solution, and the reaction solution was proceeded at room temperature under stirring for 3 hours. The reaction solution was concentrated under reduced pressure (water bath temperature was less than 30° C.), and the crude product was purified by column chromatography (petroleum ether) to obtain the product 2-(bromomethyl)-1,3,5-trifluorobenzene (25 g, yield: 60%, triphenylphosphine contained, colorless liquid).
Step 2: the solution of 2-(bromomethyl)-1,3,5-trifluorobenzene (25 g, 111 mmol) in ethanol (150 mL) was added dropwise to hydrazine hydrate (111 g, 2220 mmol) at 60° C. After the addition was completed, the temperature was maintained and the reaction was proceeded for 1 hour. The reaction solution was concentrated to dryness under reduced pressure and extracted with ethyl acetate. The crude product was purified by column chromatography (dichloromethane/methanol=20/1) to obtain the product (2,4,6-trifluorobenzyl)hydrazine (5.8 g, yield: 31%, colorless viscous liquid). ES-API: [M+H]⁺=177.0.
Step 3: (2,4,6-trifluorobenzyl)hydrazine (5.8 g, 32.7 mmol) was dissolved in acetic acid (50 mL), diethyl oxaloacetate sodium salt (5.16 g, 24.52 mmol) was added. The reaction liquid was heated to 95° C. and the reaction was proceeded under stirring for 16 hours. The reaction solution was directly concentrated under reduced pressure to remove the solvent, and the residue was slurried with ethyl acetate. The filter cake was collected after filtration to obtain ethyl 5-hydroxy-1-(2,4,6-trifluorobenzyl)-1H-pyrazole-3-carboxylate (6.0 g, yield: 76%. white solid). ES-API: [M+H]⁺=301.0.
Step 4: ethyl 5-hydroxy-1-(2,4,6-trifluorobenzyl)-1H-pyrazole-3-carboxylate (6.0 g, 20 mmol) was suspended in phosphorus oxychloride (60 mL), DMF (6 mL) was added dropwise. The reaction solution was heated to 90° C. and the reaction was proceeded under stirring for 16 hours. The reaction solution was directly concentrated under reduced pressure to remove phosphorus oxychloride, and the residue was diluted with dichloromethane and added dropwise to ice water. The mixture was extracted with dichloromethane. The organic phase was concentrated under reduced pressure. The crude product was purified by column chromatography (petroleum ether/ethyl acetate=5/1) to obtain the product ethyl 5-chloro-4-formyl-1-(2,4,6-trifluorobenzyl)-1H-pyrazole-3-carboxylate (5.0 g, yield: 71%, white solid). ES-API: [M+H]⁺=347.0.
Step 5: methoxymethyl triphenylphosphine chloride (591 mg, 1.73 mmol) was suspended in tetrahydrofuran (8 mL) under nitrogen protection, cooled to −50° C., and LDA solution (0.73 mL, 2.0 M in THF) was added dropwise. After the addition was completed, the reaction was proceeded at −20° C. under stirring for 30 minutes. The reaction solution was cooled to −50° C. again, and the solution of ethyl 5-chloro-4-formyl-1-(2,4,6-trifluorobenzyl)-1H-pyrazole-3-carboxylate (200 mg, 0.58 mmol) in tetrahydrofuran (2 mL) was added dropwise, and then stirred at room temperature for 16 hours. Then, ethyl 5-chloro-4-formyl-1-(2,4,6-trifluorobenzyl)-1H-pyrazole-3-carboxylate (3.6 g) was casted again. The reaction solution was quenched with saturated ammonium chloride solution, extracted with ethyl acetate, and the organic phase was concentrated under reduced pressure. The crude product was purified by column chromatography (petroleum ether/ethyl acetate=5/1) to obtain ethyl 5-chloro-4-(2-methoxyvinyl)-1-(2,4,6-trifluorobenzyl)-1H-pyrazole-3-carboxylate (800 mg, yield: 21%, white solid). ES-API: [M+H]⁺=375.0.
Step 6: ethyl 5-chloro-4-(2-methoxyvinyl)-1-(2,4,6-trifluorobenzyl)-1H-pyrazole-3-carboxylate (350 mg, 0.94 mmol) was dissolved in tetrahydrofuran (4 mL), and hydrochloric acid (6N, 4 mL) was added. The reaction solution was proceeded at 60° C. under stirring for 1 hour. The reaction solution was extracted with ethyl acetate, and the organic phase was concentrated under reduced pressure to obtain ethyl 5-chloro-4-(2-oxyethyl)-1-(2,4,6-trifluorobenzyl)-1H-pyrazole-3-carboxylate (460 mg, crude product, yellow liquid). The crude product was directly used in the next reaction. ES-API: [M+H]⁺=361.0.
Step 7: ethyl 5-chloro-4-(2-oxyethyl)-1-(2,4,6-trifluorobenzyl)-1H-pyrazole-3-carboxylate (460 mg, 1.27 mmol) was dissolved in methanol (2 mL), a solution of 1,1-difluoropropane-2-amine in tetrahydrofuran (4 mL) was added. The reaction solution was proceeded at room temperature under stirring for 20 minutes, and borane-2-methylpyridine complex (205 mg, 1.92 mmol) was added. The reaction solution was proceeded at room temperature under stirring for 3 hours. The reaction solution was extracted with ethyl acetate, and the organic phase was concentrated under reduced pressure to obtain ethyl 5-chloro-4-(2-(((1,1-difluoropropan-2-yl)amino)ethyl)-1-(2,4,6-trifluorobenzyl)-1H-pyrazole-3-carboxylate (530 mg, crude product, brown liquid). The crude product was directly used in the next step of reaction. ES-API: [M+H]⁺=440.1.
Step 8: ethyl 5-chloro-4-(2-((1,1-difluoropropan-2-yl)amino)ethyl)-1-(2,4,6-trifluorobenzyl)-1H-pyrazole-3-carboxylate (530 mg, 1.2 mmol) was dissolved in tetrahydrofuran/methanol/water (2 mL/1 mL/0.5 mL), and lithium hydroxide monohydrate (76 mg, 1.81 mmol) was added. The reaction solution was proceeded at room temperature under stirring for 16 hours. The reaction solution was adjusted to pH=6-7 with hydrochloric acid (2N) and directly concentrated under reduced pressure to obtain 5-chloro-4-(2-(1,1-difluoropropan-2-yl)amino)ethyl)-1-(2,4,6-trifluorobenzyl)-1H-pyrazole-3-carboxylic acid (800 mg, crude product, brown liquid). The crude product was directly used in the next reaction. ES-API: [M+H]⁺=412.0.
Step 9: 5-chloro-4-(2-(1,1-difluoropropan-2-yl)amino)ethyl)-1-(2,4,6-trifluorobenzyl)-1H-pyrazole-3-carboxylic acid (800 mg, 1.94 mmol) was dissolved in dichloromethane (5 mL), and N,N-diisopropylethylamine (3 mL) and HATU (1.1 g, 2.91 mmol) were added. The reaction solution was stirred at room and reacted for 2 hours. The reaction solution was extracted with dichloromethane, washed with brine, and the organic phase was concentrated under reduced pressure. The crude product was purified by preparative high-performance liquid chromatography (column: Ultimate XB-C18, 50*250 mm, 10 um; A: purified water B: pure acetonitrile, flow rate: 80 ml/min, wavelength: 210 nm) to obtain 3-chloro-6-(1,1-difluoropropan-2-yl)-2-(2,4,6-trifluorobenzyl)-2,4,5,6-tetrahydro-7H-pyrazolo[3,4-c]pyridin-7-one (Z22, 90 mg, yield: 24%, white solid). ¹H NMR (400 MHz, CDCl₃) δ 6.68 (t, J=8.2 Hz, 2H), 5.88 (d, J=2.6 Hz, 1H), 5.40 (s, 2H), 5.06-4.89 (m, 1H), 3.58 (t, J=6.5 Hz, 2H), 2.73 (q, J=6.4 Hz, 2H), 1.33 (d, J=7.1 Hz, 3H). ES-API: [M+H]⁺=394.1

### Example 23: Preparation of 3-chloro-2-(2,6-difluorobenzyl)-6-(tetrahydro-2H-pyran-4-yl)-2,4,5,6-tetrahydro-7H-pyrazolo[3,4-c]pyridin-7-one (Z23)

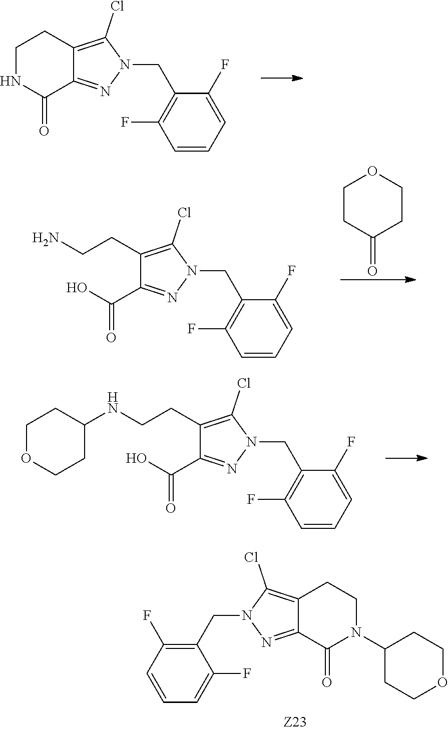 [0369] Step 1: 3-chloro-2-(2,6-difluorobenzyl)-2,4,5,6-tetrahydro-7H-pyrazolo[3,4-c]pyridin-7-one (200 mg, 0.67 mmol) was suspended in methanol/tetrahydrofuran/water (1 mL/0.5 mL/0.3 mL), and sodium hydroxide (40 mg, 1.0 mmol) was added. The reaction was proceeded at 50° C. under stirring for 2 hours. The reaction solution was adjusted to pH=6-7 with hydrochloric acid, and directly concentrated under reduced pressure to obtain crude product 4-(2-aminoethyl)-5-chloro-1-(2,6-difluorobenzyl)-1H-pyrazole-3-carboxylic acid (213 mg, crude product). The crude product was directly used in the next reaction. ES-API: [M+H]⁺=316.09.
Step 2: 4-(2-aminoethyl)-5-chloro-1-(2,6-difluorobenzyl)-1H-pyrazole-3-carboxylic acid (213 mg, 0.67 mmol) was suspended in 5 mL methanol, dihydro-2H-pyran-4(3H)-one (100 mg, 1.01 mmol) was added and the reaction was proceeded at room temperature under stirring for 30 minutes, borane-2-methylpyridine complex (110 mg, 1.01 mmol) was added. The reaction was proceeded at room temperature under stirring overnight, The reaction solution was directly concentrated under reduced pressure to obtain crude product 5-chloro-1-(2,6-difluorobenzyl)-4-(2-((tetrahydro-2H-pyran-4-yl)amino)ethyl)-1H-pyrazole-3-carboxylic acid (268 mg, crude). The crude product was directly used in the next reaction. ES-API: [M+H]⁺=400.1.
Step 3: 5-chloro-1-(2,6-difluorobenzyl)-4-(2-((tetrahydro-2H-pyran-4-yl)amino)ethyl)-1H-pyrazole-3-carboxylic acid (268 mg, 0.67 mmol) was suspended in 5 mL of dichloromethane, N,N-diisopropylethylamine (103 mg, 0.80 mmol) and HATU (380 mg, 1.00 mmol) were added, and the reaction was proceeded at room temperature under stirring for 3 hours. The reaction solution was washed with 0.5N dilute hydrochloric acid and brine, extracted with dichloromethane, and the organic phase was concentrated under reduced pressure. The crude product was purified by high performance liquid chromatography (column: Ultimate XB-C18, 50*250 mm, 10 um; A: purified water B: pure Acetonitrile flow rate: 80 ml/min, wavelength: 210 nm) to obtain 3-chloro-2-(2,6-difluorobenzyl)-6-(tetrahydro-2H-pyran-4-yl)-2,4,5,6-tetrahydro-7H-pyrazolo[3,4-c]pyridin-7-one (Z23, 22.2 mg, Y: 11%, white solid). ¹H NMR (400 MHz, CDCl₃) δ 7.26 (d, J=7.4 Hz, 1H), 6.86 (t, J=8.0 Hz, 2H), 5.43 (s, 2H), 4.88 (t, J=12.1 Hz, 1H), 3.98 (dd, J=12.0, 4.5 Hz, 2H), 3.59-3.35 (m, 4H), 2.67 (t, J=6.5 Hz, 2H), 1.74 (dt, J=15.5, 7.6 Hz, 2H), 1.58 (d, J=12.4 Hz, 2H). ES-API: [M+H]⁺=382.09.

### Example 24: Preparation of 3-bromo-2-(2,6-difluorobenzyl)-6-(1,1-difluoropropan-2-yl)-2,4,5,6-tetrahydro-7H-pyrazolo[3,4-c]pyridin-7-one (Z24) and Isomers Thereof <CWU-Call number

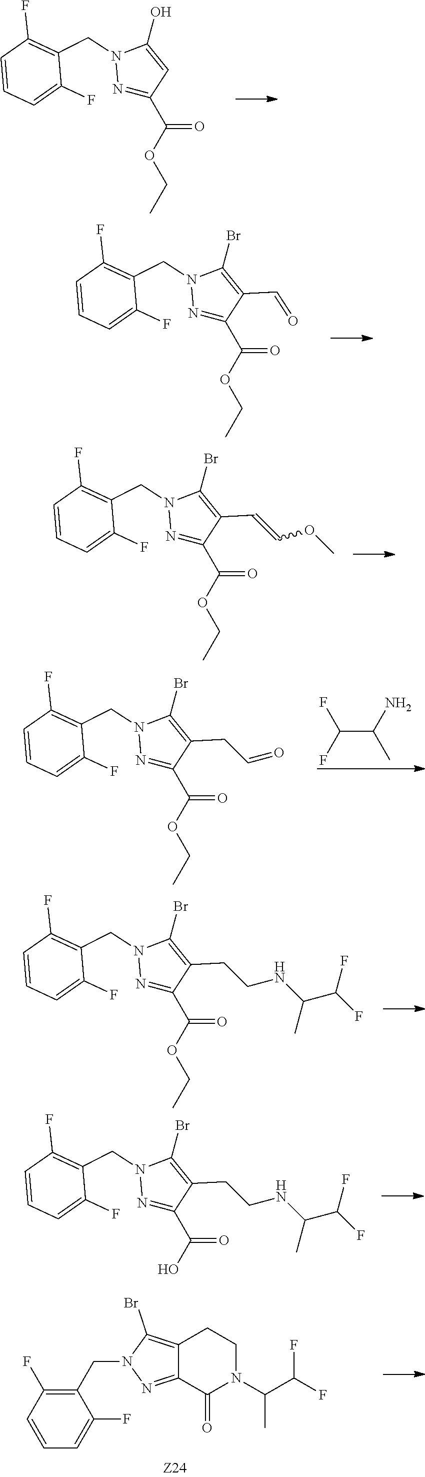 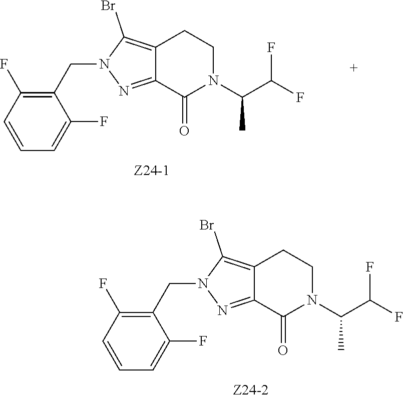 [SMILES: C[C@H](C(F)F)N1CCc2c(nn(Cc3c(F)cccc3F)c2Br)C1=O ; C[C@@H](C(F)F)N1CCc2c(nn(Cc3c(F)cccc3F)c2Br)C1=O] [0373] Step 1: ethyl 1-(2,6-difluorobenzyl)-5-hydroxy-1H-pyrazole-3-carboxylate (500 mg, 1.77 mmol) was dissolved in 1,2-dichloroethane (8 mL), then phosphorus oxybromide (1 g, 3.5 mmol) and N,N-dimethylformamide (0.25 mL) were added, the temperature was raised to 90° C. and the reaction was proceeded for 3 hours, the reaction solution was cooled to room temperature, and phosphorus oxybromide (2.4 g, 8.3 mmol) was added, heated to 90° C. and the reaction was proceeded overnight, After the reaction was completed, the reaction solution was added in ice water to quench, extracted with ethyl acetate, dried, evaporated to dryness, and purified by column chromatography (petroleum ether/ethyl acetate=4/1) to obtain the product ethyl 5-bromo-1-(2,6-difluorobenzyl)-4-formyl-1H-pyrazole-3-carboxylate (230 mg, yield: 35%, yellow solid). ES-API: [M+1]⁺=373.0.
Step 2: (methoxymethyl)triphenylphosphonium chloride (254 mg, 0.74 mmol) was suspended in tetrahydrofuran (3 mL), cooled to-10° C., and potassium tert-butoxide (0.7 mL, 1M of tetrahydrofuran solution) was added dropwise. After stirring for 20 minutes, a solution of ethyl 5-bromo-1-(2,6-difluorobenzyl)-4-formyl-1H-pyrazole-3-carboxylate (230 mg, 0.62 mmol) in tetrahydrofuran (1 mL) was added dropwise. The reaction solution was gradually raised to room temperature and the reaction was proceeded for 1 hour. After the reaction was completed, water was added to quench, the reaction solution was extracted with ethyl acetate, dried with anhydrous sodium sulfate, evaporated to dryness, and purified by thin layer chromatography (petroleum ether/ethyl acetate=4/1) to obtain the product ethyl 5-bromo-1-(2,6-difluorobenzyl)-4-(2-methoxyvinyl)-1H-pyrazole-3-carboxylate (120 mg, yield: 48%, white solid). ES-API: [M+1]⁺=401.0.
Step 3: ethyl 5-bromo-1-(2,6-difluorobenzyl)-4-(2-methoxyvinyl)-1H-pyrazole-3-carboxylate (120 mg, 0.3 mmol) was dissolved in tetrahydrofuran (1 mL), 6N HCl (0.3 mL) was added, heated to 50° C. and the reaction was proceeded for 3 hours. After the reaction was completed, water and ethyl acetate were added for extraction, dried with anhydrous sodium sulfate, and evaporated to dryness to obtain the crude product ethyl 5-bromo-1-(2,6-difluorobenzyl)-4-(2-oxyethyl)-1H-pyrazole-3-carboxylate, which was used directly in the next reaction. ES-API: [M+1]⁺=387.3.
Step 4: ethyl 5-bromo-1-(2,6-difluorobenzyl)-4-(2-oxyethyl)-1H-pyrazole-3-carboxylate (crude product, 0.3 mmol) was dissolved in methanol (1 mL), 1,1-difluoropropan-2-amine (1 mL, methanol solution) was added, stirred at room temperature for 20 minutes, then 2-methylpyridineborane complex (48 mg, 0.45 mmol) was added, the reaction was proceeded at room temperature under stirring overnight, Then 1,1-difluoropropan-2-amine (1 mL, methanol solution) and 2-methylpyridine borane complex (48 mg, 0.45 mmol) were added, heated to 50° C. and the reaction was proceeded overnight.

After the reaction was completed, the reaction solution was extracted by adding water and ethyl acetate, dried with anhydrous sodium sulfate, evaporated to dryness, and purified by thin layer chromatography (petroleum ether/ethyl acetate=2/1) to obtain the product ethyl 5-bromo-1-(2,6-difluorobenzyl)-4-(2-((1,1-difluoropropan-2-yl)amino)ethyl)-1H-pyrazole-3-carboxylate (66 mg, yield: 47%, white solid). ES-API: [M+1]⁺=466.0.
Step 5: ethyl 5-bromo-1-(2,6-difluorobenzyl)-4-(2-((1,1-difluoropropan-2-yl)amino)ethyl)-1H-pyrazole-3-carboxylate (66 mg, 0.14 mmol) was dissolved in tetrahydrofuran/methanol/water (1 mL/0.5 mL/0.3 mL), lithium hydroxide monohydrate (9 mg, 0.21 mmol) was added, and the reaction was proceeded at room temperature overnight. Then lithium hydroxide monohydrate (9 mg, 0.21 mmol) was added additionally and the reaction was proceeded at room temperature for 2 hours. After the reaction was completed, 1N HCl was added to neutralize the reaction solution, and concentrated to dryness to obtain the crude product 5-bromo-1-(2,6-difluorobenzyl)-4-(2-((1,1-difluoropropane-2-yl)amino)ethyl)-1H-pyrazole-3-carboxylic acid, which was used directly in the next reaction. ES-API: [M+1]⁺=438.0.
Step 6: 5-bromo-1-(2,6-difluorobenzyl)-4-(2-((1,1-difluoropropan-2-yl)amino)ethyl)-1H-pyrazole-3-carboxylic acid (crude product, 0.14 mmol) was dissolved in dichloromethane (3 mL), and HATU (81 mg, 0.21 mmol) and N,N-diisopropylethylamine (0.5 mL) were added in sequence. The reaction was proceeded at room temperature for 2 hours. After the reaction was completed, the solution was extracted with dichloromethane and water, the organic phase was dried over anhydrous sodium sulfate, spin-dried, and purified by thin layer chromatography (petroleum ether/ethyl acetate=1/1, Rf=0.5) to obtain the product 3-bromo-2-(2,6-difluorobenzyl)-6-(1, 1-difluoropropan-2-yl)-2,4,5,6-tetrahydro-7H-pyrazolo[3,4-c] pyridin-7-one (Z24, 19.1 mg, yield: 32%, white solid). ¹H NMR (400 MHz, CDCl₃) δ 7.33-7.28 (m, 1H), 6.90 (t, J=7.9 Hz, 2H), 5.88 (t, J=56.8 Hz, 1H), 5.49 (s, 2H), 5.05-4.91 (m, 1H), 3.58 (t, J=6.6 Hz, 2H), 2.77-2.66 (m, 2H), 1.33 (d, J=7.3 Hz, 3H). ES-API: [M+1]⁺=420.0.
Step 7: Compound (Z24) 3-bromo-2-(2,6-difluorobenzyl)-6-(1,1-difluoropropan-2-yl)-2,4,5,6-tetrahydro-7H-pyrazolo[3,4-c]pyridin-7-one (12 mg) obtained above was chiral separated (co-solvent: n-hexane: ethanol=80:20)); column: AS-H (4.6*100 mm 5 um); flow rate: 1.0 ml/min; column temperature: 30° C.) to obtain two isomer compounds. The structure of one compound was arbitrarily designated as (R)-3-bromo-2-(2,6-difluorobenzyl)-6-(1,1-difluoropropan-2-yl)-2,4,5,6-tetrahydro-7H-pyrazolo[3,4-c]pyridin-7-one (retention time: 8.819 min) (Z24-1, 4.1 mg, purity: 100%, ee value: 100%)). ES-API: [M+H]⁺=420.0. The structure of the other compound was arbitrarily designated as (S)-3-bromo-2-(2,6-difluorobenzyl)-6-(1,1-difluoropropan-2-yl)-2,4,5,6-tetrahydro-7H-pyrazolo[3,4-c]pyridin-7-one (retention time: 11.972 min) (Z24-2, 4.2 mg, purity: 100%, ee value: 100%). ES-API: [M+H]⁺=420.0.

### Example 25: Preparation of 3-chloro-2-(2,6-difluorobenzyl)-6-(2,2-dimethyltetrahydro-2H-pyran-4-yl)-2,4,5,6-tetrahydro-7H-pyrazolo[3,4-c]pyridin-7-one (Z25)

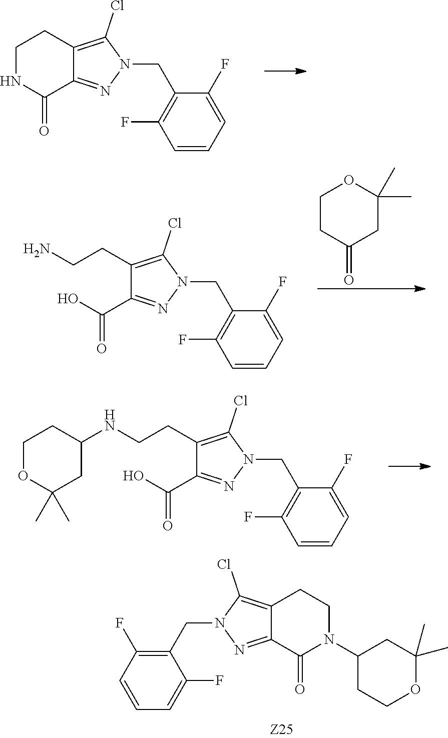 [0382] Step 1: 3-chloro-2-(2,6-difluorobenzyl)-2,4,5,6-tetrahydro-7H-pyrazolo[3,4-c]pyridin-7-one (100 mg, 0.33 mmol) was suspended in methanol/tetrahydrofuran/water (0.5 mL/0.3 mL/0.1 mL), and sodium hydroxide (60 mg, 1.5 mmol) was added. The reaction was proceeded at 50° C. under stirring for 3 hours. The reaction solution was adjusted to pH=6-7 with hydrochloric acid, and directly concentrated under reduced pressure to obtain crude product 4-(2-aminoethyl)-5-chloro-1-(2,6-difluorobenzyl)-1H-pyrazole-3-carboxylic acid (200 mg, crude). The crude product was directly used in the next reaction. ES-API: [M+1]⁺=316.1.
Step 2: 4-(2-aminoethyl)-5-chloro-1-(2,6-difluorobenzyl)-1H-pyrazole-3-carboxylic acid (crude product, 0.33 mmol) was suspended in 3 mL of methanol, 2,2-dimethyldihydro-2H-pyran-4(3H)-one (63 mg, 0.49 mmol) was added and the reaction was proceeded at room temperature under stirring for 30 minutes, borane-2-methylpyridine complex (53 mg, 0.49 mmol) was added. The reaction solution was heated to 50° C. and the reaction was proceeded overnight. The reaction solution was directly concentrated under reduced pressure to obtain the product 5-chloro-1-(2,6-difluorobenzyl)-4-(2-((2,2-dimethyltetrahydro-2H-pyran-4-yl)amino)ethyl)-1H-pyrazole-3-carboxylic acid (crude product), which was directly used in the next reaction. ES-API: [M+1]⁺=428.3.
Step 3: 5-chloro-1-(2,6-difluorobenzyl)-4-(2-((2,2-dimethyltetrahydro-2H-pyran-4-yl)amino)ethyl 1H-pyrazole-3-carboxylic acid (crude product, 0.33 mmol) was suspended in 15 mL of dichloromethane, and N,N-diisopropylethylamine (128 mg, 1.0 mmol) and HATU (188 mg, 0.49 mmol) were added, the reaction was proceeded at room temperature under stirring for 3 hours. The reaction solution was washed with 0.5N dilute hydrochloric acid and brine, extracted with dichloromethane, and the organic phase was concentrated under reduced pressure. The crude product was purified by high performance liquid chromatography (column: Ultimate XB-C18, 50*250 mm, 10 um; A: purified water B: pure acetonitrile, flow rate: 80 ml/min, gradient: within 50 minutes, B/A=20%-90%, wavelength: 214 nm, column temperature: room temperature), to obtain the product 3-chloro-2-(2,6-difluorobenzyl)-6-(2,2-dimethyltetrahydro-2H-pyran-4-yl)-2,4,5,6-tetrahydro-7H-pyrazolo[3,4-c]pyridin-7-one (Z25, 53.7 mg, yield: 40%, white solid). ¹H NMR (400 MHz, CDCl₃) δ 7.33-7.27 (m, 1H), 6.89 (t, J=8.1 Hz, 2H), 5.45 (s, 2H), 5.13-5.04 (m, 1H), 3.84-3.74 (m, 2H), 3.44 (t, J=6.0 Hz, 2H), 2.69 (t, J=6.0 Hz, 2H), 1.71-1.50 (m, 4H), 1.31 (s, 3H), 1.25 (s, 3H). ES-API: [M+H]⁺=410.1.

### Example 26: Preparation of 6-acetyl-3-chloro-2-(2,6-difluorobenzyl)-2,4,5,6-tetrahydro-7H-pyrazolo[3,4-c]pyridin-7-one(Z26)

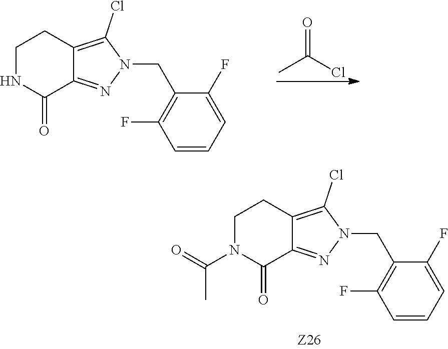 [0386] Step 1: (3-chloro-2-(2,6-difluorobenzyl)-2,4,5,6-tetrahydro-7H-pyrazolo[3,4-c]pyridin-7-one 100 mg, (0.34 mmol) was dissolved in dichloromethane (5 mL), and triethylamine (2 mL), DMAP (catalytic amount) and acetyl chloride (1 mL) were added. The reaction was proceeded at 45° C. under stirring for 4 hours. The reaction solution was quenched with ice water, extracted with dichloromethane, and concentrated under reduced pressure. The crude product was purified by preparative high performance liquid chromatography (column: Ultimate XB-C18, 50*250 mm, 10 um; A: purified water B: pure acetonitrile, flow rate: 80 ml/min, gradient: within 50 minutes, B/A=20%-90%, wavelength: 214 nm, column temperature: room temperature) to obtain 6-acetyl-3-chloro-2-(2,6-difluorobenzyl)-2,4,5,6-tetrahydro-7H-pyrazolo[3,4-c]pyridin-7-one(Z26, light yellow solid, 40.8 mg, yield: 36%). The above preparation methods were referred to prepare the following compounds by changing some raw materials:

| | MS |
|---|---|
| Structure and number | [M + H]⁺ |
| 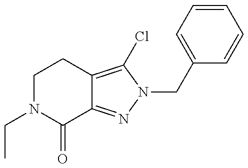 [SMILES: CCN1CCc2c(nn(Cc3ccccc3)c2Cl)C1=O] | 290.1 |
| Z27 | |
| 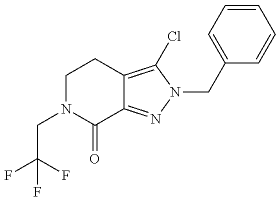 [SMILES: O=C1c2nn(Cc3ccccc3)c(Cl)c2CCN1CC(F)(F)F] | 344.1 |
| Z28 | |
| 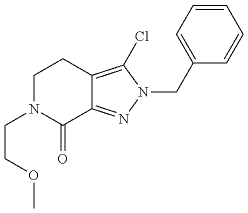 [SMILES: COCCN1CCc2c(nn(Cc3ccccc3)c2Cl)C1=O] | 320.1 |
| Z29 | |
| 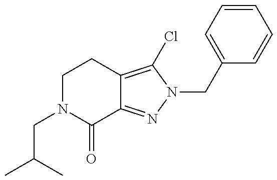 [SMILES: CC(C)CN1CCc2c(nn(Cc3ccccc3)c2Cl)C1=O] | 318.1 |
| Z30 | |
| 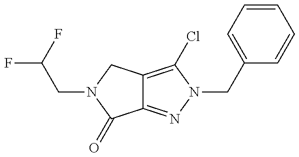 [SMILES: O=C1c2nn(Cc3ccccc3)c(Cl)c2CN1CC(F)F] | 312.1 |
| Z31 | |
| 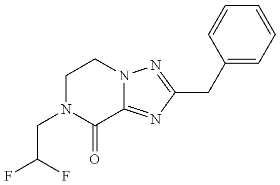 [SMILES: O=C1c2nc(Cc3ccccc3)nn2CCN1CC(F)F] | 293.1 |
| Z32 | |
| 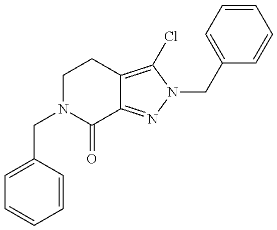 [SMILES: O=C1c2nn(Cc3ccccc3)c(Cl)c2CCN1Cc1ccccc1] | 352.1 |
| Z33 | |
| 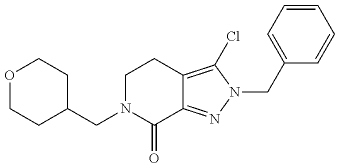 [SMILES: O=C1c2nn(Cc3ccccc3)c(Cl)c2CCN1CC1CCOCC1] | 360.1 |
| Z34 | |
| 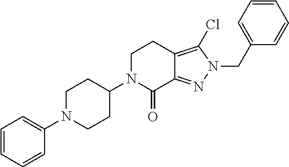 [SMILES: O=C1c2nn(Cc3ccccc3)c(Cl)c2CCN1C1CCN(c2ccccc2)CC1] | 421.2 |
| Z35 | |
| 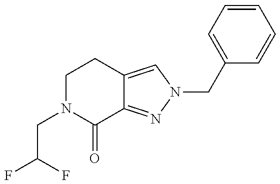 [SMILES: O=C1c2nn(Cc3ccccc3)cc2CCN1CC(F)F] | 292.1 |
| Z36 | |
| 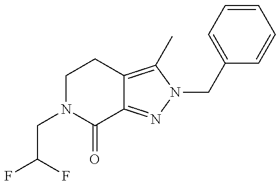 [SMILES: Cc1c2c(nn1Cc1ccccc1)C(=O)N(CC(F)F)CC2] | 306.1 |
| Z37 | |
| 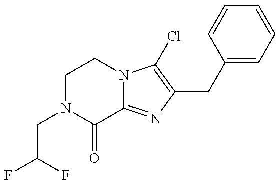 [SMILES: O=C1c2nc(Cc3ccccc3)c(Cl)n2CCN1CC(F)F] | 326.1 |
| Z38 | |
| 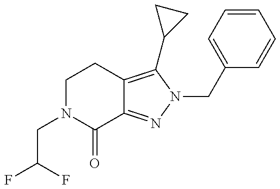 [SMILES: O=C1c2nn(Cc3ccccc3)c(C3CC3)c2CCN1CC(F)F] | 332.2 |
| Z39 | |
| 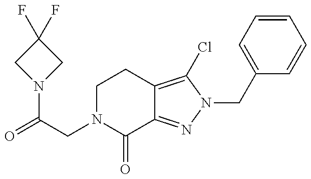 [SMILES: O=C(CN1CCc2c(nn(Cc3ccccc3)c2Cl)C1=O)N1CC(F)(F)C1] | 395.1 |
| Z40 | |
| 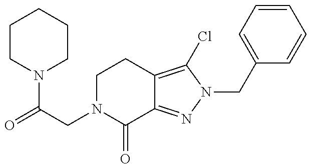 [SMILES: O=C(CN1CCc2c(nn(Cc3ccccc3)c2Cl)C1=O)N1CCCCC1] | 387.2 |
| Z41 | |
| 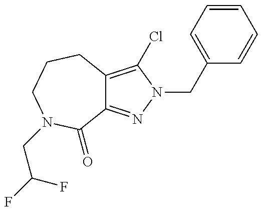 [SMILES: O=C1c2nn(Cc3ccccc3)c(Cl)c2CCCN1CC(F)F] | 340.1 |
| Z42 | |
| 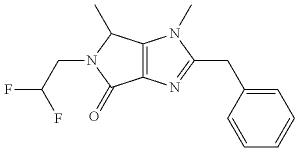 [SMILES: CC1c2c(nc(Cc3ccccc3)n2C)C(=O)N1CC(F)F] | 306.1 |
| Z43 | |
| 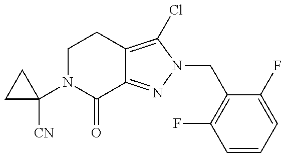 [SMILES: N#CC1(N2CCc3c(nn(Cc4c(F)cccc4F)c3Cl)C2=O)CC1] | 363.1 |
| Z44 | |
| 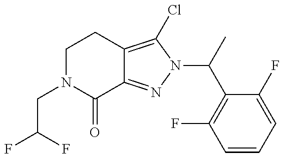 [SMILES: CC(c1c(F)cccc1F)n1nc2c(c1Cl)CCN(CC(F)F)C2=O] | 376.1 |
| Z45 | |
| 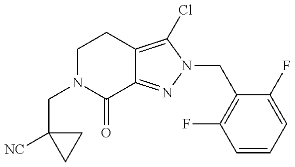 [SMILES: N#CC1(CN2CCc3c(nn(Cc4c(F)cccc4F)c3Cl)C2=O)CC1] | 377.1 |
| Z46 | |
| 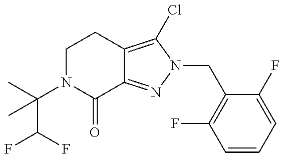 [SMILES: CC(C)(C(F)F)N1CCc2c(nn(Cc3c(F)cccc3F)c2Cl)C1=O] | 390.1 |
| Z47 | |
| 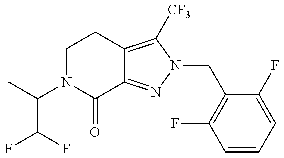 [SMILES: CC(C(F)F)N1CCc2c(nn(Cc3c(F)cccc3F)c2C(F)(F)F)C1=O] | 410.1 |
| Z48 | |
| 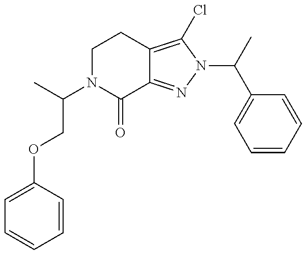 [SMILES: CC(COc1ccccc1)N1CCc2c(nn(C(C)c3ccccc3)c2Cl)C1=O] | 396.1 |
| Z49 | |
| 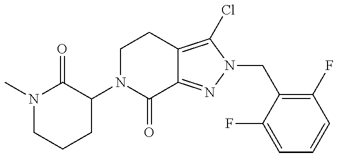 [SMILES: CN1CCCC(N2CCc3c(nn(Cc4c(F)cccc4F)c3Cl)C2=O)C1=O] | 409.1 |
| Z50 | |

### Biological Test

The U937 cell line used in the following test examples was from ATCC, number: CRL-1593.2, batch number: 63479999, culture medium: RPMI-1640+10% FBS.

The L₉₂₉ cell line used in the following test examples was from ATCC, number: CCL-1, batch number: 70001022, culture medium: MEM+10% FBS+1% PS.

The reagents used, suppliers and product numbers of the reagents were as follows: RPMI-1640, Gibco, 11875-093; FBS, Gibco, 10099-141; Trypsin-EDTA, Gibco, 25200-072; PS, Gibco, 15140-122; CellTiter Glo , Progema, G7573; DMSO, VWR AMRESCO, 0231-500ML; TNF-α protein (human, recombinant), Peprotech, 300-01A; Q-VD-Oph, MCE, HY-12305; V-shaped bottom plate, Corning, 3894; 384-well low-flange white flat-bottom microplate, Corning, 3570; RIPK1, Eurofins, 16-022; MOPS, BDH, 441644J; EDTA, Sigma, E5134; Myelin basic protein, Sigma, M1891-25.00 MG; Magnesium acetate, Merck, DU008026; ATP (non-radioactive label), Sigma, A-7699; ATP (radioactive label), Hartmann Analytic, DU008054; Phosphoric acid, Metlab, DU003000; Z-VAD: Shanghai Yuang Chemical Industry, YA02401.

### Test Example 1: Inhibitory Activity of Compounds against Programmed Necrosis of Cell Induced by TNF-α

The compounds to be tested were dissolved in DMSO and diluted with DMSO into a series of concentration gradients. 5000 U937 cells/well were seeded in a 384-well white plate. Compounds of corresponding concentrations were added to each well and mixed evenly with the cells. At the same time, human TNF-a and Q-VD-Oph were added to induce programmed necrosis of the cells. The cells were placed at 37° C., 5% CO₂ incubator for 48 hours. CellTiter-Glo reagent was used for detection, and the chemiluminescence reading was detected using a microplate reader after sufficient lysis reaction. The test results were calculated using the formula SR (%)=(RLU compound−RLU blank)/(RLU high control−RLU blank)×100%. The survival rate and the final concentration of the corresponding compound were drawn into a curve and fitted using four parameters. Inhibitory IC₅₀ on TNF-α-induced cell necrosis of the compounds were calculated. It can be seen from the experimental results that the exemplary compounds of the present invention possessed high inhibitory activity on U937 cells, with an IC₅₀ value of less than 10 μM, or less than 1 M, or less than 500 nM (for example, 0.1 nM to 500 nM); IC₅₀ values of some compounds were even less than 100 nM (for example, 0.1 nM to 100 nM) or less than 50 nM (for example, 0.1 nM to 50 nM). The experimental results of some compounds were shown in table 1:

TABLE 1
Inhibitory activity of compounds on U937 cells

| Compound number | U937 IC50 (μM) |
|---|---|
| Z1 | 0.461 |
| Z2 | 0.279 |
| Z3 | 0.129 |
| Z4 | 1.361 |
| Z5 | 0.205 |
| Z6 | 0.482 |
| Z7 | 1.828 |
| Z8 | 0.253 |
| Z9 | 0.0571 |
| Z9-1 | 0.256 |
| Z9-2 | 0.0428 |
| Z10 | 0.676 |
| Z11 | 1.603 |
| Z12 | 1.38 |
| Z13 | 0.120 |
| Z14 | 0.982 |
| Z15 | 0.127 |
| Z16 | 2.947 |
| Z17 | 1.816 |
| Z18 | 0.194 |
| Z19 | 0.168 |
| Z20 | 0.300 |
| Z21 | 0.534 |
| Z22 | 0.222 |
| Z23 | 0.311 |
| Z24 | 0.0489 |
| Z24-1 | 0.119 |
| Z24-2 | 0.0297 |
| Z25 | 0.464 |
| Z26 | 0.361 |

### Test Example 2: Inhibitory Activity of Compounds against RIPK1 Enzyme

The compound to be tested was dissolved in DMSO to prepare a 10 mM stock solution, diluted 3.16 times with DMSO into a series of concentration gradients, and then MOPS pH 7.0 buffer solution was used to dilute 50 times to prepare a working solution, and mixed well with 36 nM RIPK1 (final concentration), 0.33 mg/ml substrate MBP. Afterwards, 10 mM magnesium ions and 155 μM phosphorus 33 isotope-labeled ATP were added to react, The final concentration of DMSO was 2%. After the reaction was proceeded at room temperature for 2 hours, phosphoric acid was added to terminate. The final reaction system was processed and detected using a liquid scintillation counter. After the test results are subtracted from the blank control and compared with the reading value of the control group to convert to the activity percentage, which was plotted as a curve with the final concentration of the corresponding compound, and four-parameter fitting was used to obtain the IC₅₀ of the compound that inhibits RIPK1 enzyme activity. It can be seen from the experimental results that the exemplary compounds of the present invention possessed high inhibitory activity against RIPK1, with an IC₅₀ value of less than 200 nM (for example, 0.1 nM to 200 nM); IC₅₀ values of some compounds were even less than 100 nM (for example. 0.1 nM to 100 nM) or less than 50 nM (for example, 0.1 nM to 50 nM). The experimental results of some of the compounds were shown in Table 2:

TABLE 2
Inhibitory activity of compounds against RIPK1 enzyme

| Compound number | RIPK1 enzyme IC₅₀(nM) |
|---|---|
| Z1 | 65 |
| Z2 | 30 |
| Z3 | 48 |
| Z9-2 | 89 |
| Z42 | 184 |

### Test Example 3: Inhibitory Activity of Compounds on TNF-α-Induced Programmed Necrosis of L₉₂₉ Cells

The compounds to be tested were dissolved in DMSO to prepare a 10 mM stock solution, and diluted 3.16 times with DMSO into a series of concentration gradients, and then dilute 100 times to prepare a working solution. 10000 L₉₂₉ cells/well were seeded in a 384-well white plate. Compounds of corresponding concentrations were added to each well and mixed evenly with the cells. At the same time, 30 ng/ml mouse TNF-α and 15 μM Z-VAD were added to induce programmed necrosis of the cells, the final concentration of DMSO was 0.2%. The cells were placed at 37° C., 5% CO₂ incubator for 6 hours. CellTiter-Glo reagent was used for detection, and the chemiluminescence reading was detected using a microplate reader after sufficient lysis reaction. The test results were calculated using the formula SR (%)=(RLU compound−RLU blank)/(RLU high control−RLU blank)×100%. The survival rate and the final concentration of the corresponding compound were drawn into a curve and fitted using four parameters. Inhibitory IC₅₀ on TNF-α-induced cell necrosis of the compounds were calculated. It can be seen from the experimental results that the exemplary compounds of the present invention possessed high inhibitory activity on L₉₂₉ cells, with an IC₅₀ value of less than 1 μM, or less than 500 nM (for example, 0.1 nM to 500 nM). The experimental results of some compounds were shown in table 3:

TABLE 3
Inhibitory activity of compounds on L929 cells

| Compound number | L929 IC₅₀(μM) |
|---|---|
| Z9 | 0.6004 |
| Z9-2 | 0.4498 |
| Z24-2 | 0.3479 |

All documents mentioned in the present invention were incorporated by reference in the present application to the same extent as if each individual document was individually incorporated by reference. In addition, it should be understood that after reading the above teaching content of the present invention, those skilled in the art can make various changes or modifications to the present invention, and these equivalent forms also fall within the scope defined by the appended claims of the present application.

1. A heterocyclic lactam compound or a pharmaceutically acceptable salt, stereoisomer, solvate or prodrug thereof, the structure of the compound is as shown in formula (I): 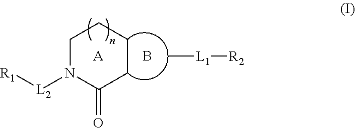
wherein,
R₁ is hydrogen, deuterium, halogen, amino, hydroxyl, cyano, nitro, C₁₋₆ alkyl, C₂₋₆ alkenyl, C₂₋₆ alkynyl, C₁₋₆ alkoxy, C₃₋₁₂ cycloalkyl, 3- to 14-membered heterocycloalkyl, C₆₋₁₄ aryl or 5- to 14-membered heteroaryl; the amino, C₁₋₆ alkyl, C₂₋₆ alkenyl, C₂₋₆ alkynyl, C₁₋₆ alkoxy, C₃₋₁₂ cycloalkyl, 3- to 14-membered heterocycloalkyl, C₆₋₁₄ aryl, 5- to 14-membered heteroaryl is optionally substituted by 1, 2, 3 or 4 substituent(s) independently selected from the group S of substituents;
R₂ is C₆₋₁₄ aryl, 5- to 14-membered heteroaryl, C₃₋₁₂ cycloalkyl or 3- to 14-membered heterocycloalkyl; wherein, the C₆₋₁₄ aryl, 5- to 14-membered heteroaryl, C₃₋₁₂ cycloalkyl or 3- to 14-membered heterocycloalkyl is optionally substituted by 1, 2, 3 or 4 group(s) independently selected from the group S of substituents;
Li is one bond, O, —S—, —S(O)—, —SO₂—, —NR₃—, —(C(R₄R₅))ₘ₁—, —C(R₄R₅)—O—, —C(R₄R₅)—S—, —C(R₄R₅)—NH—; wherein m1 is 1 or 2; R₃ is H or C₁₋₆ alkyl; each R₄ and R₅ are the same or different, and are each independently H, hydroxyl, halogen, C₁₋₆ alkyl or halogenated C₁₋₆ alkyl; or R₄ and R₅ on one of the carbon atoms and the carbon atom connected to R_{4,} R₅ together form a cyclopropyl, cyclobutyl or cyclopentyl;
L₂ is one bond, —(C(R₆R₇))ₘ₂—, —C(R₆R₇)—C(═O)— or —(C(R₆R₇))ₘ₂—O—; wherein m2 is 1 or 2; each R₆ and R₇ are the same or different, and are each independently H, hydroxyl, halogen, C₁₋₆ alkyl, halogenated C₁₋₆ alkyl or C₃₋₁₂ cycloalkyl; or R₆, R₇ on one of the carbon atoms and the carbon atoms connected to R₆, R₇ together form cyclopropyl, cyclobutyl or cyclopentyl;
the condition is that when R₁ is a C₆₋₁₄ aryl group or a 5- to 14-membered heteroaryl group, L₂ is not one bond;
ring A and ring B are fused to form a bicyclic ring system; n is 0, 1 or 2; ring A is a 5- to 7-membered nitrogen-containing heterocyclic ring; ring B is a 5- to 6-membered heteroaromatic ring; wherein, ring A is optionally substituted by 1, 2, 3 or 4 group(s) independently selected from the group S of substituents; ring B is optionally substituted by 1, 2, 3 or 4 group(s) independently selected from the group S of substituents;
the group S of substituents include: hydrogen, deuterium, halogen, nitro, cyano, oxo, hydroxyl, carboxyl, C₁₋₆ alkyl, C₂₋₆ alkenyl, C₂₋₆ alkynyl, C₁₋₆ alkoxy, C₁₋₆ alkylthio, C₃₋₁₂ cycloalkyl, C3-10 cycloalkenyl, C₆₋₁₄ aryl, 5- to 14-membered heteroaryl, 3- to 14-membered heterocycloalkyl, C₆₋₁₄ aryl-O—, C₆₋₁₄ aryl-C14 alkyl-O—, 5- to 14-membered heteroaryl-O—, 3- to 14-membered heterocycloalkyl-O—, C₁₋₆ alkyl-C(═O)O—, C₆₋₁₄ aryl-C(═O)O—, C₁₋₆ alkoxy-C(═O)O—, 5- to 14-membered heteroaryl-C(═O)O—, 3- to 14-membered heterocycloalkyl-C(═O)O—, N(R₈R₉)—C(═O)O—, N(R₈R₉)—C(═O)O—, C₁₋₆ alkyl-sulfonyloxy, C₆₋₁₄ aryl-sulfonyloxy, formyl, C₁₋₆ alkyl-C(═O)—, C₆₋₁₄ aryl-C(═O)—, 5- to 14-membered heteroaryl-C(═O)—, 3- to 14-membered heterocycloalkyl-C(═O)—, C₁₋₆ alkoxy-C(═O)—, C₆₋₁₄ aryl-OC(═O)—, C₆₋₁₄ aryl-C1-4 alkyl-O-C(═O)—, N(R₈R₉)—C(═O)—, N(R₈R₉)—C(=S)—, C₁₋₆ alkylsulfonyl, C₆₋₁₄ arylsulfonyl, 5- to 14-membered heteroarylsulfinyl, C₁₋₆ alkylsulfinyl, C₆₋₁₄ arylsulfinyl, 5- to 14-membered heteroarylsulfinyl, —N(R₈R₉); wherein, R₈ and R₉ are each independently H, C₁₋₆ alkyl, halogenated C₁₋₆ alkyl, C₆₋₁₄ aryl, 5- to 14-membered heteroaryl, 3- to 14-membered heterocycloalkyl, C₆₋₁₄ aryl C₁₋₄ alkyl-, formyl, C₁₋₆ alkyl-C(═O)—C₆₋₁₄ aryl-C(═O)—, C₁₋₆ alkoxy-C(═O)—, C₆₋₁₄ aryl-C1-4 alkyl-O—C(═O)—, C₁₋₆ alkylsulfonyl, C₆₋₁₄ arylsulfonyl; the C₁₋₆ alkyl, C₂₋₆ alkenyl, C₂₋₆ alkynyl, C₁₋₆ alkoxy, C₁₋₆ alkylthio, C₃₋₁₂ cycloalkyl, C₃₋₁₀ cycloalkenyl, C₆₋₁₄ aryl, 5- to 14-membered heteroaryl, 3- to 14-membered heterocycloalkyl is optionally substituted by one or more groups selected from the following group: halogen, hydroxyl, cyano, C₁₋₆ alkyl, halogenated C₁₋₆ alkyl.

2. The compound or a pharmaceutically acceptable salt, stereoisomer, solvate or prodrug thereof of claim 1, wherein the compound having the structure of the compound shown in formula (I-1), formula (I-2), formula (I-3), formula (I-4) or formula (I-5): 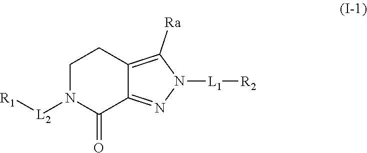
wherein, R₁, L₁, R₂, and L₂ are as defined in formula (I); Ra is selected from the group in the group S of substituents; the group S of substituents are defined as in claim 1; 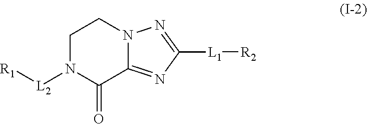
wherein, R₁, L₁, R₂, and L₂ are as defined in the formula (I); 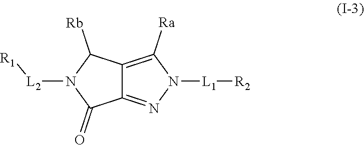
wherein, R₁, L₁, R₂, and L₂ are as defined in formula (I); Ra and Rb are selected from the group in the group S of substituents; the group S of substituents are defined as in claim 1; 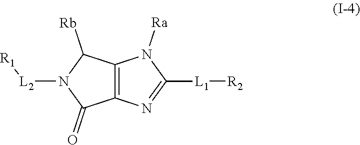
wherein, R₁, L₁, R₂, and L₂ are as defined in formula (I); Ra and Rb are selected from the group in the group S of substituents; the group S of substituents are defined as in claim 1; 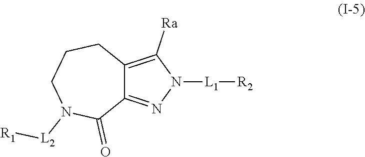
wherein, R₁, L₁, R₂, and L₂ are as defined in formula (I); Ra and Rb are selected from the group in the group S of substituents; the group S of substituents are defined as in claim 1.

3-6. (Canceled)

7. The compound or a pharmaceutically acceptable salt, stereoisomer, solvate or prodrug thereof of claim 1, wherein when L₂ is one bond, R₁ is a C₃₋₁₂ cycloalkyl group or a 3- to 14-membered heterocycloalkyl group; the C₃₋₁₂ cycloalkyl group or the 3- to 14-membered heterocycloalkyl group is optionally substituted by 1, 2, 3 or 4 substituent(s) independently selected from the group S of substituents; the group S of substituents are defined as in claim 1.

8. The compound or a pharmaceutically acceptable salt, stereoisomer, solvate or prodrug thereof of claim 1, wherein when L₂ is —(C(R₆R₇))ₘ₂—, —C(R₆R₇)—C(═O)— or (C(R₆R₇))ₘ₂—O—, R₁ is hydrogen, deuterium, halogen, amino, hydroxyl, cyano, nitro, C₁₋₆ alkyl, C₂₋₆ alkenyl, C₂₋₆ alkynyl, C₁₋₆ alkoxy, C₃₋₁₂ cycloalkyl, 3- to 14-membered heterocycloalkyl, C₆₋₁₄ aryl or 5- to 14-membered heteroaryl; the amino, C₁₋₆ alkyl, C₂₋₆ alkenyl, C₂₋₆ alkynyl, C₁₋₆ alkoxy, C₃₋₁₂ cycloalkyl, 3- to 14-membered heterocycloalkyl, C₆₋₁₄ aryl, 5- to 14-membered heteroaryl is optionally substituted by 1, 2, 3 or 4 substituent(s) independently selected from the group S of substituents; R₆, R₇, m2, and group S of substituents are each defined as in claim 1.

9. The compound or a pharmaceutically acceptable salt, stereoisomer, solvate or prodrug thereof of claim 1, wherein ring A is piperidine, pyrrolidine, pyrroline, piperazine, tetrahydropyridine or azepane; and ring A is optionally substituted by 1, 2, 3 or 4 substituent(s) independently selected from the group S of substituents; the group S substituent are defined as in claim 1.

10. The compound or a pharmaceutically acceptable salt, stereoisomer, solvate or prodrug thereof of claim 1, wherein ring B is 5- to 6-membered nitrogen-containing monocyclic heteroaromatic ring; and ring B is optionally substituted by 1, 2, 3 or 4 substituent(s) independently selected from the group S of substituents; the group S substituent are defined as in claim 1.

11. The compound or a pharmaceutically acceptable salt, stereoisomer, solvate or prodrug thereof of claim 1, wherein ring A and ring B are fused to form a bicyclic ring system; the bicyclic ring system is selected from the group consisting of: 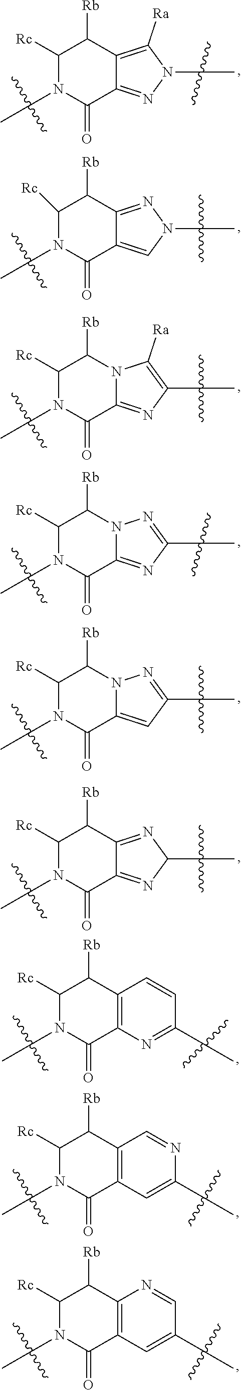 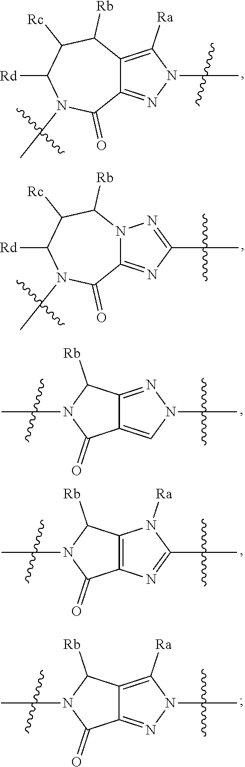
wherein Ra, Rb, Rc, Rd are groups each independently selected from the group S of substituents; the group S of substituents are defined as in claim 1.

12. The compound or a pharmaceutically acceptable salt, stereoisomer, solvate or prodrug thereof of claim 1, wherein R₂ is C₆₋₁₄ aryl; the C₆₋₁₄ aryl is phenyl, naphthyl, or 9- or 10-membered aromatic fused bicyclic ring formed by the fusion of phenyl and a non-aromatic ring; the non-aromatic ring is a 3- to 6-membered saturated or partially unsaturated monocyclic heterocycloalkyl group or a 3- to 6-membered saturated or partially unsaturated monocyclic cycloalkyl group; wherein the 3- to 6-membered saturated or partially unsaturated monocyclic heterocycloalkyl is selected from: aziridine, oxirane, azetidine, azetidin-2-one, oxetane, oxetan-2-one, oxazolidine, pyrrolidin-2-one, pyrrolidin-2,5-dione, 1,3-dioxolane, dihydrofuran-2(3H)-one, dihydrofuran-2,5-dione, piperidin-2-one, piperidin-2,6-dione, tetrahyro-2H-pyran-2-one, imidazolidine, tetrahydrofuran, tetrahydrothiophene, tetrahydropyrrole, 1,3-dioxolan-2-one, oxazolidin-2-one, imidazolidine-2-one, piperidine, piperazine, piperazin-2-one, morpholine, morpholin-3-one, morpholin-2-one, thiomorpholin-3-one 1,1-dioxide, thiomorpholine, thiomorpholine-1,1-dioxide, tetrahydropyran, 1,2-dihydroazacyclobutadiene, 1,2-dihydroazacyclobutadiene, 2,5-dihydro-1H-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, 1,3-oxazinane, hexahydropyrimidine, 1,4-dioxane, tetrahydropyrimidin-2(1H)-one, 1,4-dioxan-2-one, 5,6-dihydro-2H-pyran-2-one; the 3- to 6-membered saturated or partially unsaturated monocyclic cycloalkyl is selected from cyclopropyl, cyclobutyl, cyclopentyl, cyclopentenyl, cyclohexyl, cyclohexenyl, cyclohexdienyl, cyclobutanone, cyclobutan-1,2-dione, cyclopentanone, cyclopentan-1,3-dione, cyclohexanone, cyclohexan-1,3-dione; the phenyl, naphthyl or 9- or 10-membered aromatic fused bicyclic ring is unsubstituted or substituted by 1, 2, 3 or 4 substituent(s) each independently selected from the group S; the group S substituents are defined as in claim 1.

13. The compound or a pharmaceutically acceptable salt, stereoisomer, solvate or prodrug thereof of claim 1, wherein R₂ is 5- to 14-membered heteroaryl; the 5- to 14-membered heteroaryl is a 5- or 6-membered monocyclic heteroaryl; the 5- or 6-membered monocyclic heteroaryl is unsubstituted or substituted by 1, 2, 3 or 4 substituent(s) each independently selected from the group S of substituents; the group S of substituents are defined as in claim 1.

14. The compound or a pharmaceutically acceptable salt, stereoisomer, solvate or prodrug thereof of claim 1, wherein R₂ is 5- to 14-membered heteroaryl; the 5- to 14-membered heteroaryl is a 9- or 10-membered bicyclic heteroaryl formed by the fusion of phenyl and a 5- or 6-membered monocyclic heteroaryl; the 9- or 10-membered bicyclic heteroaryl is selected from: benzoxazole, benzisoxazole, benzimidazole, benzothiazole, benzisothiazole, benzotriazole, benzofuran, benzothiophene, indole, indazole, isoindole, quinoline, isoquinoline, quinazoline, quinoxaline, cinnoline; the 9- or 10-membered bicyclic heteroaryl is unsubstituted or substituted by 1, 2, 3 or 4 substituent(s) each independently selected from the group S of substituents; the group S of substituents are defined as in claim 1.

15. The compound or a pharmaceutically acceptable salt, stereoisomer, solvate or prodrug thereof of claim 1, wherein R₂ is 5- to 14-membered heteroaryl; the 5- to 14-membered heteroaryl is a 8- to 10-membered bicyclic heteroaryl formed by the fusion of a 5- or 6-membered monocyclic heteroaryl and a 5- or 6-membered monocyclic heteroaryl; the 8- to 10-membered bicyclic heteroaryl is unsubstituted or substituted by 1, 2, 3 or 4 substituent(s) each independently selected from the group S of substituents; the group S of substituents are defined as in claim 1.

16. The compound or a pharmaceutically acceptable salt, stereoisomer, solvate or prodrug thereof of claim 1, wherein R₂ is 5- to 14-membered heteroaryl; the 5- to 14-membered heteroaryl is a 8- to 10-membered bicyclic heteroaryl formed by the fusion of a 5- or 6-membered monocyclic heteroaryl and a non-aromatic ring; the 8- to 10-membered bicyclic heteroaryl is unsubstituted or substituted by 1, 2, 3 or 4 substituent(s) each independently selected from the group S of substituents; the group S of substituents are defined as in claim 1; the non-aromatic ring is a 3- to 6-membered saturated or partially unsaturated monocyclic heterocycloalkyl group or a 3- to 6-membered saturated or partially unsaturated monocyclic cycloalkyl group; wherein the 3- to 6-membered saturated or partially unsaturated monocyclic heterocycloalkyl is selected from aziridine, oxirane, azetidine, azetidin-2-one, oxetane, oxetan-2-one, oxazolidine, pyrrolidin-2-one, pyrrolidin-2,5-dione, 1,3-dioxolane, dihydrofuran-2(3H)-one, dihydrofuran-2,5-dione, piperidin-2-one, piperidin-2,6-dione, tetrahyro-2H-pyran-2-one, imidazolidine, tetrahydrofuran, tetrahydrothiophene, tetrahydropyrrole, 1,3-dioxolan-2-one, oxazolidin-2-one, imidazolidine-2-one, piperidine, piperazine, piperazin-2-one, morpholine, morpholin-3-one, morpholin-2-one, thiomorpholin-3-one 1,1-dioxide, thiomorpholine, thiomorpholine-1,1-dioxide, tetrahydropyran, 1,2-dihydroazacyclobutadiene, 1,2-dihydrooxetadiene, 2,5-dihydro-1H-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, 1,3-oxazinane, hexahydropyrimidine, 1,4-dioxane, tetrahydropyrimidin-2(1H)-one, 1,4-dioxan-2-one, 5,6-dihydro-2H-pyran-2-one; the 3- to 6-membered saturated or partially unsaturated monocyclic cycloalkyl is selected from cyclopropyl, cyclobutyl, cyclopentyl, cyclopentenyl, cyclohexyl, cyclohexenyl, cyclohexdienyl, cyclobutanone, cyclobutan-1,2-dione, cyclopentanone, cyclopentan-1,3-dione, cyclohexanone, cyclohexan-1,3-dione.

17. The compound or a pharmaceutically acceptable salt, stereoisomer, solvate or prodrug thereof of claim 1, wherein R₂ is 5- or 6-membered heterocycloalkyl; the 5- or 6-membered heterocycloalkyl is unsubstituted or substituted by 1, 2, 3 or 4 substituent(s) each independently selected from the group S of substituents; the group S of substituents are defined as in claim 1.

18. The compound or a pharmaceutically acceptable salt, stereoisomer, solvate or prodrug thereof of claim 1, wherein L₁ is —CH₂—, —CH₂CH₂—, —CH(CF₃)— or —CH(CH₃)—.

19. The compound or a pharmaceutically acceptable salt, stereoisomer, solvate or prodrug thereof of claim 1, wherein R₁-L₂ is selected from the group consisting of:

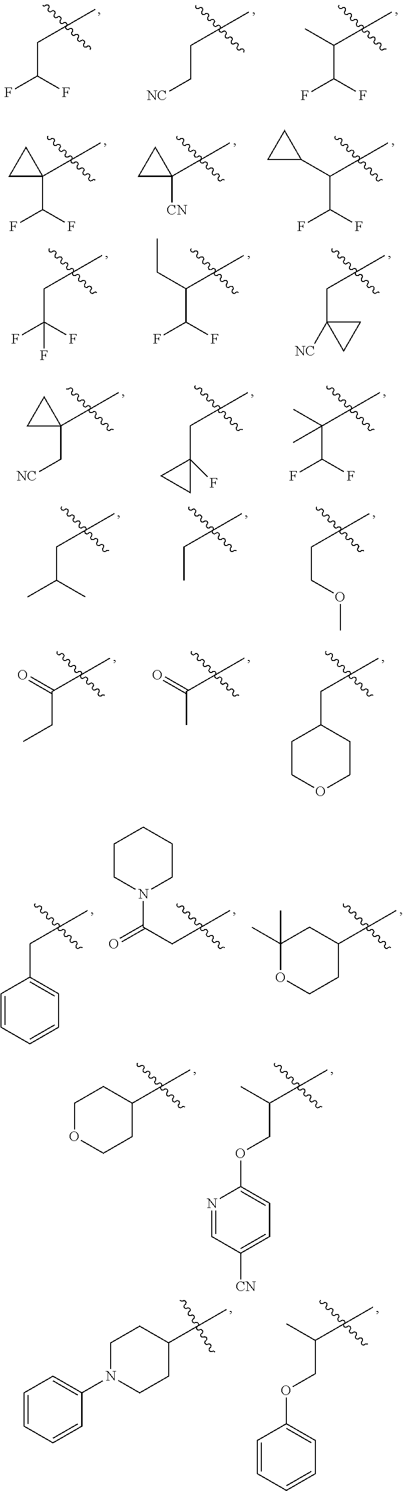

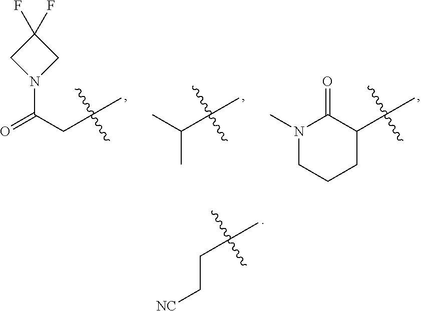

20. The compound or a pharmaceutically acceptable salt, stereoisomer, solvate or prodrug thereof of claim 1, the compound of formula (I) is selected from the compounds in the Table A.

21. A pharmaceutical composition comprising the compound of claim 1 or a pharmaceutically acceptable salt, stereoisomer, solvate or prodrug thereof; and pharmaceutically acceptable carrier.

22. A method of treating and/or preventing a disease comprising administering the compound of claim 1 or a pharmaceutically acceptable salt, stereoisomer, solvate or prodrug thereof or a pharmaceutical composition, the pharmaceutical composition comprising the compound of a claim 1 or a pharmaceutically acceptable salt, stereoisomer, solvate or prodrug thereof, the disease is selected from stroke, inflammatory bowel disease, ulcerative colitis, Crohn's disease, psoriasis, rheumatoid arthritis, NASH and heart failure.

23. A method of use of the compound of claim 1 or a pharmaceutically acceptable salt, stereoisomer, solvate or prodrug thereof or a pharmaceutical composition in preparing RIPK1 selective inhibitors, the pharmaceutical composition comprising the compound of a claim 1 or a pharmaceutically acceptable salt, stereoisomer, solvate or prodrug thereof, the RIPK1 selective inhibitors are used in treating RIPK1 related diseases or conditions.

24. The preparation method of the compound of the claim 1 or a pharmaceutically acceptable salt, stereoisomer, solvate or prodrug thereof, wherein the preparation method includes steps selected from the following schemes: 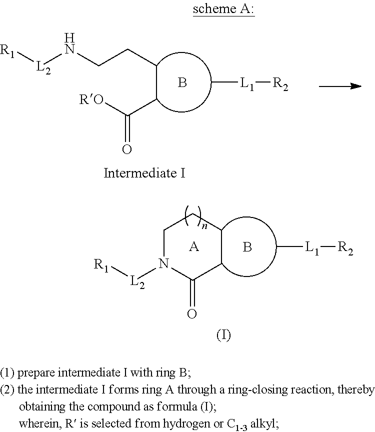
or [chemical structure]
